(19) United States
(12) Patent Application Publication
(10) Pub. No.: US 2016/0355838 A1
(43) Pub. Date: Dec. 8, 2016

(54) ANAEROBIC GERMINATION-TOLERANT PLANTS AND RELATED MATERIALS AND METHODS

(71) Applicant: International Rice Research Institute, Los Banos, Laguna (PH)

(72) Inventors: Endang M. Septiningsih, College Station, TX (US); Tobias Kretzschmar, Dennweiler-Frohnbach (DE)

(21) Appl. No.: 15/102,311

(22) PCT Filed: Dec. 11, 2014
(86) PCT No.: PCT/IB14/66818

§ 371 Date: Jun. 7, 2016

Related U.S. Application Data
(60) Provisional application No. 61/914,956, filed on Dec. 11, 2013.

(51) Int. Cl. C12N 15/82; C12N 9/16; A01H 1/04; C12Q 1/68

(52) U.S. Cl. CPC C12N 15/8267; C12Q 1/6895; C12N 9/16; A01H 1/04; C12Y 301/03012; C12Q 2600/13; C12Q 2600/158

(57) ABSTRACT

The present invention provides methods and materials useful for improving early vigor of plants during germination. The methods and materials described herein are useful for improving early vigor of plants grown under either aerobic or anaerobic conditions. In particular embodiments described herein, the methods and materials described herein are useful for improving anaerobic germination of plants.

### CROSS REFERENCE TO RELATED APPLICATIONS

This application claims the priority benefit of U.S. Provisional Application No. 61/914,956, filed Dec. 11, 2013, the entire disclosure of which is expressly incorporated herein by reference for all purposes.

### STATEMENT REGARDING GOVERNMENT FUNDING

This invention was not made with United States Government support.

### STATEMENT REGARDING SEQUENCE LISTING

The Sequence Listing, filed electronically and identified as 53-55490-IRRI-13-007_SL.txt, was created on Dec. 8, 2014, is 176,093 bytes in size and is hereby incorporated by reference.

### BACKGROUND OF THE INVENTION

Oryza sativa, rice, is a staple food for more than half of the world population. Demand for food is estimated to double by 2050, and thus increasing rice production is paramount to sustain global food security.

Rice is often grown by germinating seeds in a controlled environment and then transplanting seedlings to the field. This method of planting, especially manual transplanting, demands significant labor. It can take up to 30 person-days to transplant a single hectare. Rice seedlings must be grown in a nursery, the establishment and maintenance of which is in itself labor intensive. Seedlings are pulled and transplanted into puddle and leveled fields 15 to 40 days after seeding (DAS).

Direct seeding on puddle soil, or wet-seeding, is a method of planting rice wherein the seeds are sown directly onto puddled soil. Rice seed must be often pre-germinated by soaking the seed for approximately 24 hours prior to incubation for up to 36 hours, thereby increasing associated costs. If seed is not pre-germinated, germinating seed often fails to survive the hypoxic environment resulting from being completely submerged.

Global socio-economic developments are creating strong incentives for rice farmers to shift from transplanting to direct-seeded practices as a means of intensification and economization (REF).

Thus it would be beneficial to identify the gene or genes underlying anaerobic germination (AG) tolerance, which would enable uniform germination and seedling establishment under complete submergence. The identification of these genes or genes would allow for the development of AG-tolerant plants, including rice.

### SUMMARY OF THE INVENTION

Described herein are methods and materials useful for improving early vigor during germination in plants. In particular, the present disclosure provides isolated nucleic acids associated with improved tolerance to anaerobic germination. The disclosure further provides recombinant DNA for the generation of transgenic plants with tolerance to anaerobic germination, transgenic plant cells, and methods of producing the same. The present disclosure further provides methods for generating transgenic seed that can be used to produce a transgenic plant having improved tolerance to anaerobic germination, and methods for improving tolerance to anaerobic germination in a plant involving marker assisted selection and backcrossing.

In a particular embodiment described herein is a method for improving early vigor during germination of plants, comprising: a) crossing a crossing plant of one plant variety having chromosomal DNA that comprises a polynucleotide sharing an identity with a polynucleotide selected from the group consisting of: SEQ ID NO: 4 (OsTPP7); SEQ ID NO: 7 (OsTPP7); SEQ ID NO:8 (OsTPP7); and SEQ ID NO: 9 (OsTPP7) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with a recipient plant of a distinct plant variety having chromosomal DNA that does not include the polynucleotide, thereby producing one or more progeny plants; and b) selecting one or more progeny plants having chromosomal DNA that comprises the polynucleotide, wherein the selected one or more progeny plants have improved early vigor during germination

In another embodiment described herein, a polynucleotide is detected by a method selected from the group consisting of: allele-specific hybridization; Southern analysis; Northern analysis; in situ hybridization; and hybridization of primers followed by polymerase chain reaction amplification of a region of a marker.

In another embodiment described herein, the method for improving early vigor during germination of plants further comprises the steps: c) backcrossing the one or more selected progeny plants to produce backcross progeny plants; and d) selecting one or more backcross progeny plants having chromosomal DNA that comprises the polynucleotide. In yet another embodiment described herein, these steps c) and d) are repeated one or more times to produce third or higher backcross progeny plants having chromosomal DNA that comprises the polynucleotide.

In another embodiment described herein, the selected progeny plants are further selected for having tolerance of anaerobic germination. In yet another embodiment described herein, anaerobic germination occurs in conditions of complete submergence.

In another embodiment described herein, the crossing plant and recipient plant are capable of crossing, and are selected from the group of plants consisting of: rice; corn; wheat; barley; sorghum; millet; oats; rye; sunflower; canola; and soybean. In yet another embodiment described herein, the crossing plant and recipient plant are rice. And in yet another aspect, the crossing plant is a rice plant of rice variety Khao Hlan On. In yet another method described herein, he recipient plant is a rice plant selected from the group consisting of: the Indica rice group; the Japonica rice group; and the Glaberrima rice group, an in certain embodiments, the recipient plant is a rice plant of rice variety IR64.

In a particular embodiment described herein is a method for selecting a plant having improved early vigor during germination relative to a control plant, comprising: a) inducing expression or increasing expression in a plant, a polynucleotide sharing at least 70% identity with a polynucleotide selected from the group consisting of: SEQ ID NO: 4 (OsTPP7); SEQ ID NO: 7 (OsTPP7); SEQ ID NO:8 (OsTPP7); and SEQ ID NO: 9 (OsTPP7), wherein the induced or increased expression of the polynucleotide is obtained by transforming and expressing in the plant the polynucleotide; and b) selecting a plant having improved early vigor during germination relative to a control plant, wherein the plant having improved early vigor during germination relative to a control plant is selected by detecting presence of the polynucleotide in the transformed plant.

In another embodiment described herein, the selected plant is further selected for having tolerance of anaerobic germination compared to a control plant. In a particular embodiment, the selected plant is further selected for having tolerance of anaerobic germination compared to a control plant, wherein anaerobic germination occurs in conditions of complete submergence.

In another embodiment described herein, the induced or increased expression of the polynucleotide is under the control of at least one promoter functional in plants. In certain aspects, the at least one promoter and the polynucleotide are operably linked. In particular embodiments, he at least one promoter is selected from the group consisting of: a functional fragment of maize polyubiquitin promoter; and a functional fragment of native Khao Hlan On TPP7 promoter.

In another embodiment, the plant, or transgenic plant cell, is selected from the group of plants and plant cells consisting of: rice; corn; wheat; barley; sorghum; millet; oats; rye; sunflower; canola; and soybean.

In yet another embodiment, the nucleotide shares an identity selected from the group of identities consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

In a particular embodiment described herein, is a method for generating a plant having improved early vigor during germination relative to a control plant comprising: a) transforming a plant cell, plant, or part thereof with a construct comprising: 1) a polynucleotide having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with a polynucleotide selected from the group consisting of: SEQ ID NO: 4 (OsTPP7); SEQ ID NO: 7 (OsTPP7); SEQ ID NO:8 (OsTPP7); and SEQ ID NO: 9 (OsTPP7); and 2) a promoter operably linked to the polynucleotide; and b) expressing the construct in a plant cell, plant, or part thereof, thereby generating a plant having improved early vigor during germination relative to a control plant. In certain aspects, this method further comprises a step of selecting for a plant having improved tolerance of anaerobic germination relative to a control plant. In a particular embodiment, anaerobic germination occurs in conditions of complete submergence.

In a particular embodiment described herein, is a method for producing a transgenic plant having improved early vigor during germination relative to a control plant comprising: a) transforming and expressing in a plant cell at least one polynucleotide having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with a polynucleotide selected from the group consisting of: SEQ ID NO: 4 (OsTPP7); SEQ ID NO: 7 (OsTPP7); SEQ ID NO:8 (OsTPP7); and SEQ ID NO: 9 (OsTPP7); and b) cultivating the plant cell under conditions promoting plant growth and development, and obtaining transformed plants expressing OsTPP7.

In a particular embodiment described herein, is a transgenic plant cell comprising: a) at least one polynucleotide having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with a polynucleotide selected from the group consisting of: SEQ ID NO: 4 (OsTPP7); SEQ ID NO: 7 (OsTPP7); SEQ ID NO:8 (OsTPP7); and SEQ ID NO: 9 (OsTPP7); and b) at least one promoter that is functional in plants, wherein the promoter and polynucleotide are operably linked and incorporated into the plant cell chromosomal DNA.

In certain embodiments, a transgenic plant cell or transgenic plant is homozygous for the polynucleotide.

In another embodiment described herein, is a transgenic plant comprising a plurality of transgenic plant cells generated by a method described herein.

In a particular embodiment described herein, is a transgenic plant comprising: a) at least one polynucleotide having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with a polynucleotide selected from the group consisting of: SEQ ID NO: 4 (OsTPP7); SEQ ID NO: 7 (OsTPP7); SEQ ID NO:8 (OsTPP7); and SEQ ID NO: 9 (OsTPP7); and b) at least one promoter that is functional in plants, wherein the promoter and polynucleotide are operably linked and incorporated into the chromosomal DNA of one or more plant cells of the transgenic plant. Also described herein are seed and plant parts of a transgenic plant described herein.

In a particular embodiment described herein, is a method for selecting transgenic plants having improved early vigor during germination relative to a control plant, comprising: a) screening a population of plants for increased vigor during germination, wherein plants in the population comprise a transgenic plant cell having recombinant DNA incorporated into its chromosomal DNA, wherein the recombinant DNA comprises a promoter that is functional in a plant cell and that is functionally linked to an open reading frame of a polynucleotide having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with a polynucleotide selected from the group consisting of: SEQ ID NO: 4 (OsTPP7); SEQ ID NO: 7 (OsTPP7); SEQ ID NO:8 (OsTPP7); and SEQ ID NO: 9 (OsTPP7), wherein individual plants in said population that comprise the transgenic plant cell exhibit at least one phenotype, when compared to a control plant, selected from the group consisting of: increased alpha amylase activity; and enhanced coleoptiles elongationenic plant cell; and b) selecting from the population one or more plants that exhibit vigor during germination greater than the vigor during germination in control plants which do not comprise the transgenic plant cell.

In a particular embodiment described herein, is an isolated polynucleotide selected from the group consisting of: a) a polynucleotide having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with a polynucleotide selected from the group consisting of: SEQ ID NO: 4 (OsTPP7); SEQ ID NO: 7 (OsTPP7); SEQ ID NO:8 (OsTPP7); and SEQ ID NO: 9 (OsTPP7); and b) a polynucleotide which is fully complementary to the polynucleotide of a), wherein the polynucleotide is operably linked to a heterologous polynucleotide.

In another embodiment described herein is a recombinant expression cassette comprising a polynucleotide described herein, wherein the polynucleotide is operably linked to a promoter.

In another embodiment described herein, the polynucleotide is fully complementary to a polynucleotide selected from the group consisting of: SEQ ID NO: 4 (OsTPP7); SEQ ID NO: 7 (OsTPP7); SEQ ID NO:8 (OsTPP7); and SEQ ID NO: 9 (OsTPP7).

### BRIEF DESCRIPTION OF THE FIGURES

The patent or application file contains at least one drawing best viewed in color.

FIGS. 1A-1D: Phenotypes of qAG-9-2 parents and NIL. FIG. 1A) Illustration of the qAG-9-2 region in KHO and IR64. Grey area depicts the INDEL region. FIG. 1B) Line graph showing means of coleptile length after 2-4 days of growth in the dark under submergence (DOGS). n=>69, +/−s.e.m., p<0.001 for all data. FIG. 1C) Line graph showing means of alpha amylase activity after 1-4 DOGS. n=4, +/−s.e.m., p<0.05 for all data. FIG. 1D) Bar graph showing coleoptiles length after 4 DOGS in the absence (−) and presence (+) of 90 mM sucrose of seedlings grown in the dark and under submergence. n=>46, +/−s.e.m. Different letters indicate difference with p<0.01.

FIGS. 2A-2B: Phenotypes of OsTPP7 transgenic lines. FIG. 2A) Bar graphs showing means of coleoptile lengths of qAG-9-2 parents (IR64, NIL, KHO), two native promoter OSTPP7 complementation lines, IR64-TPP7 (NP+) and respective null segregants (NP−), two OsTPP7 over-expression lines (OX+) and respective null segregants (OX−), an OsTPP7 T-DNA insertion line (KO) and its null segregants (KOC) after four days of growth in the dark under submergence. White columns indicate OsTPP7 absence, and grey columns indicate OsTPP7 presence. n=>80, +/−s.e.m.=p<0.05, **=p<0.01, ***=p<0.001.

FIGS. 3A-3D: Sugar metabolites related to trehalose-6 phosphate metabolism in OsTPP7 absent and present lines. Box plots showing concentrations of the T6P precursor glucose-6-phosphate (FIG. 3A), the OsTPP7 substrate trehalose-6-phosphate (FIG. 3B), the OsTPP7 product trehalose (FIG. 3C) and sucrose (FIG. 3D), for which T6P acts an indicator, per dry weight (DW) in embryos and coleoptiles of IR64, OsTPP7-containing IR64 (NP1) and NIL after 4 days of growth in the dark under submergence. Whiskers indicate minima and maxima. White boxes indicate OsTPP7 absence and grey boxes indicate OsTPP7 presence. n=5, *=p<0.05, **=p<0.01, ***=p<0.001.

FIGS. 4A-4H: Spatial-temporal expression of OsTPP7 and effects of OsTPP7 expression on global gene expression. Photographs showing OsTPP7 promoter-GUS signal in KHO seedlings grown in the dark under air for two days (FIG. 4A) and four days (FIGS. 4B-4C), seedlings grown in the dark under submergence for two days (FIG. 4D) and four days (FIGS. 4E-4F) and seedlings grown under air for six days under 16 h/day light (FIG. 4G). Insets are magnifications of the aleuron. Scale bars equal 5 mm (FIGS. 4A, 4B, 4D, and 4E) and 20 mm (FIGS. 4C, 4F, and 4G). FIG. 4H) Pie graph showing ontologies of 42 genes that are significantly up-regulated in IR64 when it is expressing OsTPP7 under control of its native promoter (see also Table 1).

FIGS. 5A-5B: Fine mapping of qAG-9-2. FIG. 5A) qAG-9-2 was delimited to a region of 100.5 kb using 260 BC₄F₄ lines from 38 NILs with different size of introgression in the region of the QTL (A-M). Solid boxes represent homozygous Khao Hlan On introgression; seedling survival represented by L (Low), M (Medium) and H (high). FIG. 5B) Second round of fine mapping using selected families (A, D, E, O, J, and K, which splits to K-1 and K-2) and additional 50 BC₄F₄ lines from 9 NILs (N-Q) narrowed down qAG-9-2 into a region of ˜58 kb. Successive dominant markers predicted a ˜20 kb deleted region in IR64.

FIGS. 6A-6B: Frequency and distribution of the OsTPP7 deletion. FIG. 6A) Frequency of the OsTPP7 deletion in a range of IRRI-derived cultivars and the IR8 parents DGWG and Peta as monitored by a set of co-dominant markers. The upper band corresponds to the presence of the deletion whereas the lower band corresponds to the absence of the deletion. FIG. 6B) Pie graph showing distribution of the OsTPP7 deletion across a rice diversity panel of 816 lines. 145 lines (18%) contained the deletion, the majority of which belonged to the Indica rice subgroup.

FIGS. 7A-B: qAG-9-2 and candidate gene analysis and qAG-9-2-dependent AG-survival phenotype. FIG. 7A) Photograph showing results of semi-quantitative RT-PCR for all genes in the qAG-9-2 candidate region on cDNA obtained from IR64 and NIL after four days of growth in the dark under air (AIR) or submergence (H2O). Alpha tubulin served as a housekeeping control and genomic DNA (GC) as a PCR control. FIG. 7B) Bar graph showing survival rates of the tolerant parent KHO, the susceptible parent IR64 and qAG-9-2 positive (+NIL) and negative (−NIL) lines after 14 days of growth under submergence. n>50, +/−s.e.m. ***=p<0.001

FIG. 8: Early seedling vigor under aerobic conditions in qAG-9-2-containing lines. Bar graph showing root and shoot length of seedlings after four days of growth in the dark under aerobic conditions. n=>209, +/−s.e.m. ***=p<0.001

FIGS. 9A-9C: Analysis of OX and NP transgenic lines. FIG. 9A) Photograph showing results of semi-quantitative RT-PCR for OsTPP7 and alpha tubulin (TUB) on cDNAs obtained from embryos and coleoptiles after four days of growth in dark and under submergence for two independent homozygous constitutive promoter OsTTP7 lines (OX HM) and their respective null segregants (OX HW), and four independent homozygous native promoter OsTTP7 lines (NP HM) and their respective null segregants (NP HW). The three bands for OsTPP7 correspond to three splice variants, with splice variant 1 (LOC_Os09g20390.1; SEQ ID NO: 7) being most dominant. FIGS. 9B-9C) Illustrations showing sites of T-DNA insertions for NP1 (9B) and NP2 (9C) as determined by sequencing and BLAST analysis of TAIL-PCR amplicons.

FIGS. 10A-10C: Genotypic analysis of a OsTPP7 T-DNA insertion (KO) line. FIG. 10A) Illustration showing sites of T-DNA insertions for the OsTPP7 KO line CLON PFG_3A-08739.L, and primer positions for determination of homozygosity. FIGS. 10B-10C) Photographs showing results of homozygosity PCR results for T2 individuals derived from 4 T1 individuals (3A-01, 3A-03 and 3A-05 were hemizygous for T-DNA insertion whereas 3A-06 was a wild type control line). “Yellow” indicates lines with homozygous T-TNA insertion alleles, “blue” indicates lines with homozygous wild type alleles (null segregants), and “green” indicates hemizygous lines for the T-DNA insertion. Primers pairs included TLBP2 (SEQ ID NO: 122) & PFGTPP-R (SEQ ID NO: 121) (FIG. 10B) and PFGTPP-F (SEQ ID NO: 120) & PFGTPP-R (SEQ ID NO: 121) (FIG. 10C) (see Table 1).

FIGS. 11A-11B: Catalytic activity of OsTPP7 in vitro. FIG. 11A) Line graph showing Michaelis Menten kintics for recombinant protein of the LOC_Os09g20390.1 (SEQ ID NO: 7) transcript, as monitored by phosphate release in relation to T6P concentration. n=6, +/−s.e.m. FIG. 11B) Hanes-Woolf plot of the same data set presented in FIG. 11A showing an apparent Km=0.2 mM by trend-line regression.

FIGS. 12A-12B: Effects of high exogenous glucose concentrations on growth of IR64 and OX lines. FIG. 12A) Photograph showing phenotypes of IR64 and two transgenic lines constitutively expressing OsTPP7 (OX) after 2 weeks growth plates containing 0.4 M glucose. FIG. 12B) Bar graph showing total fresh weight of IR64 and two OX lines after 2 weeks growth plates containing either 0.4 M glucose or 0.4 M sorbitol. n=12+/−s.d., ***=p<0.001.

FIGS. 13A-13G: OsTPP7 expression. FIGS. 13A-13F) Quantitative RT-PCR for OsTPP7 on cDNA obtained from NIL embryos (dark grey columns) and coleoptiles (light grey columns) grown for 2-4 days in dark and under submergence (H₂O) or aerobic conditions (AIR). Expression of OsTPP7 after 2-4 days under submergence shown relative to the 2 day coleoptile-embryo samples (white column) (FIG. 13A). Expression of OsTPP7 under submerged conditions relative to aerobic conditions in coleoptile-embryo samples after 2 days (FIG. 13B), in coleoptiles after 3 days (FIG. 13C), in embryos after 3 days (FIG. 13D), in coleoptiles after 4 days (FIG. 13E), and in embryos after 4 days (FIG. 13F). Polyubiquitin, ubiquitin and actin served as references. Average fold changes+/−s.e. as calculated by REST software (Qiagen) after 3000 iterations (N=3), *=p<0.05. FIG. 13G) Photograph showing results of semi-quantitative RT-PCR of OsTPP7 and alpha tubulin on cDNA obtained from leaves of 2 week old seedlings of two independent IR64 lines constitutively expressing OsTPP7 (OX), HKO, the NIL and IR64. Whereas the OX lines show expression it is absent in KHO and NIL, which contain native OsTPP7 alleles.

FIG. 14: qAG-9-2 deletion flanking region marker positioning. Bold sequence is deleted in IR64, size of deletion is 20.9 kb encompassing parts of LOC_Os08g20380 (C_{TE}; SEQ ID NO: 3), all of LOC_Os08g20390 (D; SEQ ID NO: 4; OsTPP7) and parts of LOC_Os08g20400 (E; SEQ ID NO: 5).

### DETAILED DESCRIPTION OF THE INVENTION

Throughout this disclosure, various publications, patents and published patent specifications are referenced. The disclosures of these publications, patents and published patent specifications are hereby incorporated by reference into the present disclosure to more fully describe the state of the art to which this invention pertains.

The present invention provides methods and materials useful for improving early vigor of plants during germination. The methods and materials described herein are useful for improving early vigor of plants grown under either aerobic or anaerobic conditions. In particular embodiments described herein, the methods and materials described herein are useful for improving anaerobic germination of plants.

### DEFINITIONS

In describing the present invention, the following terms will be employed and are intended to be defined as indicated below.

As used herein a “phenotypic trait” is a distinct variant of an observable characteristic, e.g., tolerance to anaerobic germination, of a plant that may be inherited by a plant or may be artificially incorporated into a plant by processes such as transfection. Early vigor during germination is one example of a phenotypic trait.

As used herein, “early vigor during germination” refers to the health of an emerging seedling. Vigor may be measured in the developing seedling's coleoptiles or radical, as indicated by a number of metrics, including alpha amylase activity, growth rates, and sugar availability in a seed or seedling's sink organs. In certain instances, early vigor during germination is determined under aerobic germination conditions. Early vigor during germination may also refer to the vigor of a seedling developing under anaerobic, or hypoxic, conditions. “Anaerobic conditions” refers to low oxygen, or hypoxic, environments, such as flooded rice fields. These anaerobic germination conditions may be simulated in a laboratory environment by partially or completely submerging germinating seeds and developing seedlings.

As used herein, “introgression” means the movement of one or more genes, or a group of genes, from one plant variety into the gene complex of another as a result of backcrossing.

As used herein a “transgenic plant” means a plant whose genome has been altered by the stable integration of recombinant DNA. A transgenic plant includes a plant regenerated from an originally-transformed plant cell and progeny transgenic plants from later generations or crosses of a transformed plant.

As used herein, “polynucleotide” includes reference to a deoxyribopolynucleotide, ribopolynucleotide, or analogs thereof. A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein.

As used herein “recombinant DNA” means DNA which has been a genetically engineered and constructed outside of a cell including DNA containing naturally occurring DNA or cDNA or synthetic DNA.

“Percent identity” describes the extent to which the sequences of DNA or protein segments are invariant throughout a window of alignment of sequences, for example nucleotide sequences or amino acid sequences. Percent identity is calculated over the aligned length preferably using a local alignment algorithm, such as BLAST. As used herein, sequences are “aligned” when the alignment produced by BLAST has a minimal e-value.

As used herein “promoter” includes reference to a region of DNA upstream from the start of transcription and involved in recognition and binding of RNA polymerase and other proteins to initiate transcription. A “promoter that is functional in a plant cell” or “plant promoter” is a promoter capable of initiating transcription in plant cells whether or not its origin is a plant cell, e.g. is it well known that Agrobacterium promoters are functional in plant cells. Thus, plant promoters include promoter DNA obtained from plants, plant viruses and bacteria such as Agrobacterium and Bradyrhizobium bacteria. A “constitutive” promoter is a promoter, which is active under most environmental conditions.

As used herein “operably linked” means the association of two or more DNA fragments in a recombinant DNA construct so that the function of one, e.g. protein-encoding DNA, is controlled by the other, e.g. a promoter. A functional linkage between a first sequence, such as a promoter and a second sequence, wherein the promoter sequence initiates and mediates transcription of the DNA corresponding to the second sequence. Generally, operably linked means that the nucleic acid sequences being linked are contiguous and, where necessary to join two protein coding regions, contiguous and in the same reading frame.

As used herein “expressed” means produced, e.g. a protein is expressed in a plant cell when its cognate DNA is transcribed to mRNA that is translated to the protein.

As used herein a “control plant” means a plant that does not contain the recombinant DNA that imparts tolerance to anaerobic germination. A control plant is used to identify and select a transgenic plant that has enhanced tolerance to anaerobic germination. A suitable control plant can be a non-transgenic plant of the parental line used to generate a transgenic plant, i.e. devoid of recombinant DNA. A suitable control plant may in some cases be a progeny of a hemizygous transgenic plant line that does not contain the recombinant DNA, known as a negative segregant.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

Recombinant DNA constructs are assembled using methods well known to persons of ordinary skill in the art and typically comprise a promoter operably linked to DNA, the expression of which provides the enhanced agronomic trait. Other construct components may include additional regulatory elements, such as 5′ leaders and introns for enhancing transcription, 3′ untranslated regions (such as polyadenylation signals and sites), DNA for transit or signal peptides.

Numerous promoters that are active in plant cells have been described in the literature. These include promoters present in plant genomes as well as promoters from other sources, including nopaline synthase (NOS) promoter and octopine synthase (OCS) promoters carried on tumor-inducing plasmids of Agrobacterium tumefaciens and the CaMV35S promoters from the cauliflower mosaic virus as disclosed in U.S. Pat. Nos. 5,164,316 and 5,322,938. Useful promoters derived from plant genes are found in U.S. Pat. No. 5,641,876 which discloses a rice actin promoter, U.S. Pat. No. 7,151,204 which discloses a maize chloroplast aldolase promoter and a maize aldolase (FDA) promoter, and US Patent Application Publication 2003/0131377 A1 which discloses a maize nicotianamine synthase promoter. Also useful in the present invention are the maize polyubiquitin promoter and the native TPP7 promoter (SEQ ID NO: 6), described below These and other promoters that function in plant cells are known to those skilled in the art and available for use in recombinant nucleic acids of the present invention to provide for expression of desired genes in transgenic plants and plant cells.

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 (see U.S. Pat. No. 5,641,876) and rice actin 2 genes, the maize alcohol dehydrogenase gene intron, the maize heat shock protein 70 gene intron (U.S. Pat. No. 5,593,874) and the maize shrunken 1 gene. See also US Patent Application Publication 2002/0192813A1 which discloses 5′, 3′ and intron elements useful in the design of effective plant expression vectors.

The term “quantitative trait locus” or “QTL” refers to a polymorphic genetic locus with at least two alleles that reflect differential expression of a continuously distributed phenotypic trait.

The term “associated with” or “associated” in the context of this invention refers to, for example, a nucleic acid and a phenotypic trait, that are in linkage disequilibrium, i.e., the nucleic acid and the trait are found together in progeny plants more often than if the nucleic acid and phenotype segregated independently.

The term “marker” or “molecular marker” or “genetic marker” refers to a genetic locus (a “marker focus”) used as a point of reference when identifying genetically linked loci such as a quantitative trait locus (QTL). The term may also refer to nucleic acid sequences complementary to the genomic sequences, such as nucleic acids used as probes or primers. The primers may be complementary to sequences upstream or downstream of the marker sequences. The term can also refer to amplification products associated with the marker. The term can also refer to alleles associated with the markers. Allelic variation associated with a phenotype allows use of the marker to distinguish germplasm on the basis of the sequence.

The term “crossed” or “cross” in the context of this invention means the fusion of gametes via pollination to produce progeny (i.e., cells, seeds or plants). The term encompasses both sexual crosses (the pollination of one plant by another) and selfing (self-pollination, i.e., when the pollen and ovule are from the same plant or from genetically identical plants).

General Description

With transplanting being the major form of rice establishment, there has been little selective pressure on tolerance to anaerobic germination (AG) in tropical rice breeding. As a wetland species, several landraces still maintain high AG tolerance, whereas in many popular varieties, this trait has been lost. This is exemplified by the loss of OsTPP7 function in International Rice Research Institute (IRRI) breeding materials. However, recent shifts from transplanting to direct seeding, largely driven by economic factors, are creating a growing demand for direct-seeded rice varieties and management practices for the tropics.

The functional characterization of OsTPP7 described herein contributes to the understanding of energy starvation responses under hypoxic stress and links trehalose metabolism to α-amylase-mediated starch utilization during germination. In addition the studies described herein provide valuable information and materials for the development of AG-tolerant lines to facilitate a progression from transplanting to direct seeding in tropical environments even for small holder farmers.

As disclosed and described herein, the KHO-derived QTL qAG-9-2 confers tolerance to AG through presence of a functional OsTPP7 allele. OsTPP7 contributes to AG tolerance by modulation of local trehalose-6-phosphate (T6P) levels. In scutellar tissues, this leads to dampening of sugar depended α-amylase repression, while in embryo, coleoptile and radicle it enhances sink strength through uncoupling of sugar status and TPP concentration.

The present invention provides, inter alia, isolated nucleic acids comprising a trehalose-6-phosphate phosphatase-encoding polynucleotide. The isolated nucleic acids of the present invention can be made using standard recombinant methods, synthetic techniques, or combinations thereof. In preferred embodiments, the nucleic acid shares at least 70% sequence identity with a with a polynucleotide having a sequence selected from the group consisting of: SEQ ID NO: 4 (OsTPP7); SEQ ID NO: 7 (OsTPP7); SEQ ID NO:8 (OsTPP7); and SEQ ID NO: 9 (OsTPP7). In other embodiments, the nucleic acid shares a sequence identity with the polynucleotide having a sequence selected from the group consisting of: SEQ ID NO: 4 (OsTPP7); SEQ ID NO: 7 (OsTPP7); SEQ ID NO:8 (OsTPP7); and SEQ ID NO: 9 (OsTPP7) selected from the group consisting of at least 75% identity; at least 80% identity; at least 85% identity; at least 90% identity; at least 95% identity; at least 96% identity; at least 97% identity; at least 98% identity; at least 99% identity; and 100% identity.

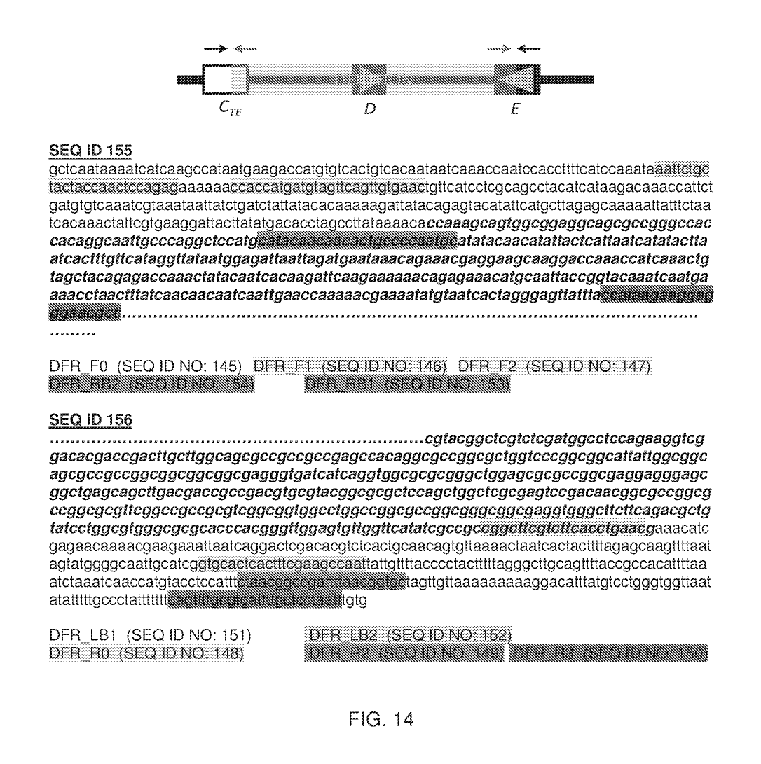

Preferably, a QTL or genes of the present invention comprises at least one marker associated with the QTL or genes of the present invention. In particular embodiments, deletion markers (forward primers) DFR_F0 (SEQ ID NO: 145), DFR_F1 (SEQ ID NO: 146), DFR_F2 (SEQ ID NO: 147), DFR_RB1 (SEQ ID NO: 153), and DFR_RB2 (SEQ ID NO: 154) may be used in conjunction with deletion markers (reverse primers) DFR_R0 (SEQ ID NO: 148), DFR_R2 (SEQ ID NO: 149), DFR_R3 (SEQ ID NO: 150), DFR_LB1 (SEQ ID NO: 151), and DFR_LB2 (SEQ ID NO: 152) to detected a deletion of approximately 20.9 kb of qAG-9-2, comprising parts of LOC_Os08g20380 (SEQ ID NO: 3), all of LOC_Os08g20390 (SEQ ID NO: 4; OsTPP7), and parts of LOC_Os08g20400 (SEQ ID NO: 5) (FIG. 14). Such a deletion is indicative of a susceptibility to anaerobic germination, as a plant having the deletion lacks the tolerance-conferring gene OsTPP7.

In yet other embodiments, the presence or lack of presence of treahalose-6-phosphate phosphatase OsTTP7 (LOC_Os08g20390) may be detected using primers designed against the nucleic acid sequences of SEQ ID NO: 4 (LOC_Os09g20390), SEQ ID NO: 7 (LOC_Os09g20390.1), SEQ ID NO: 8 (LOC_Os09g20390.2), or SEQ ID NO: 9 (LOC_Os09g20390.3). Examples of useful primers for detecting the presence or lack of presence of OsTPP7 include, but are not limited to, qTTP_L (SEQ ID NO: 129), qTPP_R (SEQ ID NO: 130), TPP_RT_F (SEQ ID NO: 137), TPP_RT_R (SEQ ID NO: 138), FLEX_SV1_F (SEQ ID NO: 109); and FLEX_SV1_R (SEQ ID NO: 110).

The nucleic acid sequence of QTL qAG-9-2 is provided in SEQ ID NO: 18 (from rice cultivar IR64; shows deletion including candidate gene OsTPP7) and SEQ ID NOs: 19-22 (truncated sequence from rice cultivar Khao Hlan On; shows presence of candidate gene OsTPP7).

The presence of a QTL or gene of the present invention may be determined by methods known to the skilled person. For instance, a nucleic acid sequence comprising the QTL or a gene thereof may be isolated from a donor plant by fragmenting the genome of said plant and selecting those fragments harboring one or more markers indicative of the QTL or gene. Subsequently, or alternatively, the marker sequences (or parts thereof) indicative of the QTL or gene may be used as PCR amplification primers, in order to amplify a nucleic acid sequence comprising said QTL or gene from a genomic nucleic acid sample or a genome fragment obtained from said plant. The amplified sequence may then be purified in order to obtain the isolated QTL or gene. The nucleotide sequence of the QTL or gene, and/or of any additional markers comprised therein, may then be obtained by standard sequencing methods.

The present invention therefore also relates to an isolated nucleic acid (preferably DNA) sequence that comprises a QTL or gene of the present invention.

In embodiments of such methods for detecting the presence of a QTL or gene in a plant, the method may also comprise the steps of providing a oligonucleotide or nucleic acid capable of hybridizing under stringent hybridization conditions to a nucleic acid sequence of a marker linked to the QTL or gene, preferably selected from the markers disclosed herein as being linked to said QTL or gene, contacting the oligonucleotide or nucleic acid with a genomic nucleic acid of a plant suspected of possessing relatively higher tolerance to anaerobic germination, and determining the presence of specific hybridization of the oligonucleotide or nucleic acid to said genomic nucleic acid.

Preferably, said method is performed on a nucleic acid sample obtained from the plant suspected of possessing relatively higher tolerance to anaerobic germination, although in situ hybridization methods may also be employed.

Production of Plants with Improved Early Vigor During Germination by Transgenic Methods.

According certain aspects of the present invention, a nucleic acid (preferably DNA) sequence comprising a trehalose-6-phosphate phosphatase-encoding polynucleotide may be used for the production of a plant with improved early vigor during germination. Preferably, the nucleic acid shares at least 70% sequence identity with any one of SEQ ID NOs: 4 and 7-9. In other embodiments, the nucleic acid shares a sequence identity with a with a polynucleotide having a sequence selected from the group consisting of: SEQ ID NO: 4 (OsTPP7); SEQ ID NO: 7 (OsTPP7); SEQ ID NO:8 (OsTPP7); and SEQ ID NO: 9 (OsTPP7), selected from the group consisting of at least 75% identity; at least 80% identity; at least 85% identity; at least 90% identity; at least 95% identity; at least 96% identity; at least 97% identity; at least 98% identity; at least 99% identity; and 100% identity.

In particular embodiments, the nucleic acid sequence may be used for production of a plant with improved tolerance of anaerobic germination. In these aspects, the invention provides for the use of a nucleic acid sequence of the present invention for producing a plant with improved tolerance to germination under hypoxic stress, wherein use involves the introduction of the nucleic acid sequence in a plant having relatively low tolerance to anaerobic germination. The nucleic acid sequence may be derived from a suitable donor plant. Rice cultivar Khao Hlan On is one example of a suitable donor plant. Other rice plants exhibiting a relatively high tolerance to anaerobic germination may also be utilized as donor plants, as the present invention describes how this material may be identified.

Once identified in a suitable donor, the nucleic acid sequence that comprises a trehalose-6-phosphate phosphatase-encoding polynucleotide may be transferred to a suitable recipient plant by any method available. In certain embodiments, a suitable recipient plant is a rice plant that does not comprise the nucleic acid sequence of the present invention. Suitable recipient plants include, but are not limited to: IR64, IR8, and approximately 20% of rice cultivars, wherein the nucleic acid sequence of the present invention has been deleted.

The nucleic acid sequence may be transferred by crossing a donor plant with a susceptible recipient plant (i.e. by introgression), by transformation, by protoplast fusion, by a doubled haploid technique, by embryo rescue, or by any other nucleic acid transfer system, optionally followed by selection of offspring plants comprising the nucleic acid and exhibiting improved early vigor during germination relative to a control plant. For transgenic methods of transfer, the nucleic acid sequence may be isolated from said donor plant by using methods known in the art and the thus isolated nucleic acid sequence may be transferred to the recipient plant by transgenic methods, for instance by means of a vector, in a gamete, or in any other suitable transfer element, such as a ballistic particle coated with the nucleic acid sequence.

Plant transformation generally involves the construction of an expression cassette that will function in plant cells, as described above. In the present invention, such a cassette comprises a nucleic acid sequence having at least 70% sequence identity with a with a polynucleotide having a sequence selected from the group consisting of: SEQ ID NO: 4 (OsTPP7); SEQ ID NO: 7 (OsTPP7); SEQ ID NO:8 (OsTPP7); and SEQ ID NO: 9 (OsTPP7), operatively linked to a regulatory element such as a promoter. In certain embodiments, the nucleic acid is operatively linked to the maize ubiquitin promoter. In other embodiments, the nucleic acid is operative linked to the native OsTPP7 promoter (SEQ ID NO: 6). The expression cassette may be in the form of a plasmid, and can be used alone or in combination with other plasmids to provide transgenic plants that have improved early vigor during germination, using transformation methods known in the art, such as the Agrobacterium transformation system.

Expression cassettes may include at least one marker gene, operably linked to a regulatory element (such as a promoter) that allows transformed cells containing the marker to be either recovered by negative selection (by inhibiting the growth of cells that do not contain the selectable marker gene), or by positive selection (by screening for the product encoded by the marker gene). Many commonly used selectable marker genes for plant transformation are known in the art, and include, for example, genes that code for enzymes that metabolically detoxify a selective chemical agent which may be an antibiotic or a herbicide, or genes that encode an altered target which is insensitive to the inhibitor. Several positive selection methods are known in the art, such as mannose selection. In a particular embodiment described herein, a β-glucuronidase (GUS) reporter system is used. Alternatively, marker-less transformation can be used to obtain plants without mentioned marker genes, the techniques for which are known in the art.

One method for introducing an expression cassette into a plant is based on the natural transformation system of Agrobacterium. A. tumefaciens and A. rhizogenes are plant pathogenic soil bacteria that genetically transform plant cells. The Ti and Ri plasmids of A. tumefaciens and A. rhizogenes, respectively, carry genes responsible for genetic transformation of the plant. Methods of introducing expression vectors into plant tissue include the direct infection or co-cultivation of plant cells with Agrobacterium tumefaciens. Descriptions of Agrobacterium vectors systems and methods for Agrobacterium-mediated gene transfer are provided by Gruber and Crosby, 1993 and Moloney et al., 1989. See also, U.S. Pat. No. 5,591,616. General descriptions of plant expression vectors and reporter genes and transformation protocols and descriptions of Agrobacterium vector systems and methods for Agrobacterium-mediated gene transfer can be found in Gruber and Crosby, 1993. General methods of culturing plant tissues are provided, for example, by Mild et al., 1993 and by Phillips, et al., 1988. A reference handbook for molecular cloning techniques and suitable expression vectors is Sambrook and Russell (2001).

Another method for introducing an expression cassette into a plant is based on microprojectile-mediated transformation, wherein DNA is carried on the surface of microprojectiles. The expression cassette is introduced into plant tissues with a biolistic device that accelerates the microprojectiles to speeds of 300 to 600 m/s, which is sufficient to penetrate plant cell walls and membranes. Another method for introducing DNA to plants is via the sonication of target cells. Alternatively, liposome or spheroplast fusion has been used to introduce expression vectors into plants. Direct uptake of DNA into protoplasts using CaCl₂ precipitation, polyvinyl alcohol, or poly-L-ornithine may also be used. Electroporation of protoplasts and whole cells and tissues has also been described.

Following transformation of target tissues, expression of the above described selectable marker genes allows for preferential selection of transformed cells, tissues and/or plants, using regeneration and selection methods now well known in the art. The markers described herein may also be used for that purpose. Selection may also occur based on phenotype, wherein plants expressing improved early vigor during germination may be selected. In particular embodiments, selection based of phenotype may occur under hypoxic germination conditions, such as complete submergence.

Synthetic Methods for Constructing Nucleic Acids

The isolated nucleic acids of the present invention can also be prepared by direct chemical synthesis by methods known in the art. Chemical synthesis generally produces a single stranded oligonucleotide. This may be converted into double stranded DNA by hybridization with a complementary sequence or by polymerization with a DNA polymerase using the single strand as a template. One of skill in the art will recognize that while chemical synthesis of DNA is limited to sequences of about 100 bases, longer sequences may be obtained by the ligation of shorter sequences.

Production of Plants with Improved Early Vigor During Germination by Programmable Site-Specific Nucleases.

Zinc-finger nucleases (ZFNs), transcription activator-like effector nucleases (TALENs), and clustered regularly interspaced short palindromic repeat (CRISPR)/Cas-based RNA-guided DNA endonucleases comprise a powerful class of tools useful in genome engineering. The chimeric nucleases of ZFNs and TALENs are composed of programmable, sequence-specific DNA-binding modules linked to a nonspecific DNA cleavage domain ZFNs and TALENs enable a broad range of genetic modifications by inducing DNA double-strand breaks that stimulate error-prone non-homologous end joining or homology-directed repair at specific genomic locations.

Site-specific nucleases induce DNA double-strand breaks that stimulate non-homologous end joining and homology directed repair at targeted genomic loci. A thorough review of the ZFN, TALEN, and CRISPR/Cas-based RNA-guided DNA endonuclease is available (Gaj et al., 2013). Further discussion of ZNFs may be found in U.S. Pat. Nos. 8,106,255, 8,399,218, and 8,592,645. Further discussion of TALENs may be found in U.S. Pat. No. 8,697,853. Further discussion of CRISPR/Cas-based RNA-guided DNA endonucleases may be found in U.S. Pat. No. 8,697,359, and in J. D. Sander & J. K. Juong (2014).

In certain aspects, any one of these technologies (ZFNs, TALENs, and CRISPR/Cas-based RNA guided DNA endonucleases) may be used to modify the genome of a plant. Such modification may include modification, insertion, or deletion of a polynucleotide.

Production of Plants with Improved Early Vigor During Germination by Non-Transgenic Methods.

In an alternative embodiment for producing a plant with improved early vigor during germination, protoplast fusion can be used for the transfer of nucleic acids from a donor plant to a recipient plant. Protoplast fusion is an induced or spontaneous union, such as a somatic hybridization, between two or more protoplasts (cells of which the cell walls are removed by enzymatic treatment) to produce a single bi- or multi-nucleate cell. The fused cell, which may even be obtained with plant species that cannot be interbred in nature, is tissue cultured into a hybrid plant exhibiting the desirable combination of traits. More specifically, a first protoplast can be obtained from a plant that exhibits improved early vigor during germination, and more particularly, improved tolerance to anaerobic germination. For example, a protoplast from rice cultivar Khao Hlan On be used. A second protoplast may be obtained from rice or other plant variety, preferably a variety that comprises commercially desirable characteristics, such as, but not limited to disease resistance, insect resistance, weed resistance, etc. The protoplasts are then fused using traditional protoplast fusion procedures, which are known in the art.

Alternatively, embryo rescue may be employed in the transfer of the nucleic acid of the present invention from a donor plant to a recipient plant. Embryo rescue can be used as a procedure to isolate embryo's from crosses wherein plants fail to produce viable seed. In this process, the fertilized ovary or immature seed of a plant is tissue cultured to create new plants.

The present invention also relates to a method of producing a plant having improved early vigor during germination comprising the steps of performing a method for detecting the presence of a quantitative trait locus (QTL) or nucleic acid sequence associated with improved early vigor during germination (particularly, improved tolerance to anaerobic germination) in a donor plant according to the invention as described above, and transferring a nucleic acid sequence comprising a trehalose-6-phosphate phosphatase-encoding polynucleotide from said donor plant to a plant having a relatively lower tolerance to anaerobic germination. The transfer of said nucleic acid sequence may be performed by any of the methods previously described herein. In particular embodiments, the detected QTL comprises a nucleic acid comprising a trehalose-6-phosphate phosphatase-encoding polynucleotide. In a preferred embodiment, the nucleic acid shares at least 70% sequence identity with a with a polynucleotide having a sequence selected from the group consisting of: SEQ ID NO: 4 (OsTPP7); SEQ ID NO: 7 (OsTPP7); SEQ ID NO:8 (OsTPP7); and SEQ ID NO: 9 (OsTPP7).

A preferred embodiment of such a method comprises the transfer by introgression of the nucleic acid sequence from a plant having improved early vigor during germination (particularly, improved tolerance to anaerobic germination) into a plant having a relatively lower early vigor during germination by crossing the plants. This transfer may thus suitably be accomplished by using traditional breeding techniques.

QTLs correlated with improved early vigor during germination are preferably introgressed into commercial plant varieties by using marker-assisted breeding (MAS). Marker-assisted breeding or marker-assisted selection involves the use of one or more of the molecular markers for the identification and selection of those offspring plants that contain one or more of the genes that encode for the desired trait. In the present instance, such identification and selection is based on selection of QTLs of the present invention or markers associated therewith. MAS can also be used to develop near-isogenic lines (NIL) harboring the QTL of interest, allowing a more detailed study of each QTL effect and is also an effective method for development of backcross inbred line (BIL) populations (see, e.g., Nesbitt et al., 2001; van Berloo et al., 2001). Plants developed according to this embodiment can advantageously derive a majority of their traits from the recipient plant, and derive improved vigor during germination, and in particular, improved tolerance of anaerobic germination, from the donor plant. Using primers such as those described herein, a single gene may be similarly used in MAS.

As discussed briefly above, traditional breeding techniques can be used to introgress a nucleic acid sequence encoding for improved early vigor during germination into a recipient plant having a relatively lower vigor during germination. In one method, which is referred to as pedigree breeding, a donor plant comprising a nucleic acid sequence encoding for improved early vigor during germination is crossed with a plant having a relatively lower early vigor during germination that preferably exhibits commercially desirable characteristics, such as, but not limited to, disease resistance, insect resistance, weed resistance, etc. The resulting plant population (representing the F1 hybrids) is then self-pollinated and set seeds (F2 seeds). The F2 plants grown from the F2 seeds are then screened for improved early vigor during germination.

Plants developed according to any one of the methods described herein may be screened for improved early vigor during germination in a number of different ways. For example, the population can be screened by observing survival of seed germinated under hypoxic conditions for 1-4 days. In a preferred embodiment, such screening and selection occurs during germination of seeds under complete submergence.

A Plant Having Improved Early Vigor During Germination, or a Part Thereof, Obtainable by a Method of the Invention, is Also an Aspect of the Present Invention.

Another aspect of the present invention relates to a plant having improved early vigor during germination, comprising within its genome the nucleic acid sharing at least 70% sequence identity with a with a polynucleotide having a sequence selected from the group consisting of: SEQ ID NO: 4 (OsTPP7); SEQ ID NO: 7 (OsTPP7); SEQ ID NO:8 (OsTPP7); and SEQ ID NO: 9 (OsTPP7), wherein the nucleic acid is not in its natural genetic background. The plants having improved early vigor during germination of the present invention can be of any genetic type such as inbred, hybrid, haploid, dihaploid, parthenocarp, or transgenic. Further, the plants of the present invention may be heterozygous or homozygous for the improved early vigor during germination trait, preferably homozygous. Although the isolated nucleic acid of the present invention may be transferred to any plant in order to provide for a plant having improved early vigor during germination, the methods and plants of the invention are preferably related to the cereal grasses family, more preferably rice.

Inbred lines having improved early vigor during germination can be developed using the techniques of recurrent selection and backcrossing, selfing and/or dihaploids or any other technique used to make parental lines. In a method of selection and backcrossing, improved early vigor during germination can be introgressed into a target recipient plant (which is called the recurrent parent) by crossing the recurrent parent with a first donor plant (which is different from the recurrent parent and referred to herein as the “non-recurrent parent”). The recurrent parent is a plant that has relatively low early vigor during germination (particularly, low tolerance of anaerobic germination) and possesses commercially desirable characteristics, such as, but not limited to disease resistance, insect resistance, weed resistance, etc.

The non-recurrent parent comprises a nucleic acid sequence that encodes for trehalose-6-phosphate phosphatase, which results in improved early vigor during germination. In preferred embodiments the nucleic acid sequence that encodes for trehalose-6-phosphate phosphatase confers improved tolerance to anaerobic germination. The non-recurrent parent can be any plant variety or inbred line that is cross-fertile with the recurrent parent. The progeny resulting from a cross between the recurrent parent and non-recurrent parent are backcrossed to the recurrent parent. The resulting plant population is then screened. The population can be screened in a number of different ways. F1 hybrid plants that exhibit improved early vigor during germination (tolerance of anaerobic germination), comprise the requisite nucleic acid sequence, and possess commercially desirable characteristics, are then selected and selfed for a number of generations in order to allow for the plant to become increasingly inbred. This process of continued selfing and selection can be performed for two to five generations, or more. The result of such breeding and selection is the production of lines that are genetically homogenous for the gene associated with improved early vigor during germination (OsTPP7) as well as other genes associated with traits of commercial interest. Instead of using phenotypic pathology screens of bioassays, MAS can be performed using one or more of the herein described molecular markers, hybridization probes or nucleic acids to identify those progeny that comprise a nucleic acid sequence encoding for improved early vigor during germination (particularly, improved tolerance of anaerobic germination). Alternatively, MAS can be used to confirm the results obtained from quantitative bioassays. Once the appropriate selections are made, the process is repeated. The process of backcrossing to the recurrent parent and selecting for improved early vigor during germination is repeated for approximately five or more generations. The progeny resulting from this process are heterozygous for one or more genes that encode for improved early vigor during germination (particularly, improved tolerance to anaerobic germination). The last backcross generation is then selfed in order to provide for homozygous pure breeding progeny for improved early vigor during germination.

The lines having improved early vigor during germination described herein can be used in additional crossings to create hybrid plants having improved early vigor during germination (particularly, improved tolerance of anaerobic germination). For example, a first inbred plant having improved early vigor during germination of the invention can be crossed with a second inbred plant possessing commercially desirable traits such as, but not limited to, disease resistance, insect resistance, weed resistance, etc. This second inbred line may or may not have relatively improved early vigor during germination.

Marker Assisted Selection and Backcrossing.

As is known to those skilled in the art, there are many kinds of molecular markers. For example, molecular markers can include restriction fragment length polymorphisms (RFLP), random amplified polymorphic DNA (RAPD), amplified fragment length polymorphisms (AFLP), single nucleotide polymorphisms (SNP) or simple sequence repeats (SSR). Simple sequence repeats (SSR) or microsatellites are regions of DNA where one to a few bases are tandemly repeated for few to hundreds of times. Simple sequence repeats are thought to be generated due to slippage mediated errors during DNA replication, repair and recombination. Over time, these repeated sequences vary in length between one cultivar and another. When SSRs occur in a coding region, their survival depends on their impact on structure and function of the encoded protein. Since repeat tracks are prone to DNA-slippage mediated expansions/deletions, their occurrences in coding regions are limited by non-perturbation of the reading frame and tolerance of expanding amino acid stretches in the encoded proteins. Among all possible SSRs, tri-nucleotide repeats or multiples thereof are more common in coding regions.

A single nucleotide polymorphism (SNP) is a DNA sequence variation occurring when a single nucleotide—A, T, C or G—differs between members of a species (or between paired chromosomes in an individual). For example, two sequenced DNA fragments from two individuals, AGCCTA and AGCTTA, contain a difference in a single nucleotide. In this case, there are two alleles: C and T.

A primary motivation for development of molecular markers in crop species is the potential for increased efficiency in plant breeding through marker assisted selection (MAS) and marker assisted backcrossing (MABC). Genetic marker alleles, or alternatively, identified QTL alleles, are used to identify plants that contain a desired genotype at one or more loci and that are expected to transfer the desired genotype, along with a desired phenotype to their progeny. Genetic marker alleles can be used to identify plants that contain a desired genotype at one locus or at several unlinked or linked loci (e.g., a haplotype) and that would be expected to transfer the desired genotype, along with a desired phenotype to their progeny. The present invention provides the means to identify plants, particularly rice, that are able to improve the early vigor during germination, and in particular, improve tolerance to anaerobic germination, by identifying plants having a specified quantitative trait locus or gene, e.g., qAG-9-2, OsTPP7, and homologous or linked markers. Similarly, by identifying plants from a cross the exhibit poor early vigor during germination, these low-vigor plants can be identified and, e.g., eliminated from subsequent crosses.

After a desired phenotype, e.g., improved early vigor during germination and improved tolerance to anaerobic germination, and a polymorphic chromosomal locus, e.g., a marker locus, QTL, or gene (e.g., OsTPP7) are determined to segregate together, it is possible to use those polymorphic loci to select for alleles corresponding to the desired phenotype: a process called marker-assisted selection (MAS). In brief, a nucleic acid corresponding to the marker nucleic acid is detected in a biological sample from a plant to be selected. This detection can take the form of hybridization of a probe nucleic acid to a marker, e.g., using allele-specific hybridization, Southern analysis, northern analysis, in situ hybridization, hybridization of primers followed by PCR amplification of a region of the marker or the like. A variety of procedures for detecting markers are described herein. After the presence (or absence) of a particular marker and/or marker allele in the biological sample is verified, the plant may be selected, i.e., used to make progeny plants by selective breeding.

Screening a large number of plants for improved early vigor during germination can be expensive, time consuming and unreliable. Use of the polymorphic loci described herein, and genetically-linked nucleic acids, as genetic markers for the early vigor during germination (anaerobic germination) locus is an effective method for selecting varieties capable of fertility restoration in breeding programs. For example, one advantage of marker-assisted selection over field evaluations for improved early vigor during germination is that MAS can be done at any time of year regardless of the growing season. Moreover, environmental effects are irrelevant to marker-assisted selection.

Another use of MAS in plant breeding is to assist the recovery of the recurrent parent genotype by backcross breeding. Backcross breeding is the process of crossing a progeny back to one of its parents. Backcrossing is usually done for the purpose of introgressing one or a few loci from a donor parent into an otherwise desirable genetic background from the recurrent parent. The more cycles of backcrossing that are done, the greater the genetic contribution of the recurrent parent to the resulting variety. This is often necessary, because donor parent plants may be otherwise undesirable. In contrast, varieties which are the result of intensive breeding programs may have excellent yield, fecundity or the like, merely being deficient in one desired trait such as tolerance to anaerobic germination. As a skilled worker understands, backcrossing can be done to select for or against a trait.

Markers corresponding to genetic polymorphisms between members of a population can be detected by numerous methods, well-established in the art (e.g., restriction fragment length polymorphisms, isozyme markers, allele specific hybridization (ASH), amplified variable sequences of the plant genome, self-sustained sequence replication, simple sequence repeat (SSR), single nucleotide polymorphism (SNP) or amplified fragment length polymorphisms (AFLP).

The majority of genetic markers rely on one or more properties of nucleic acids for their detection. For example, some techniques for detecting genetic markers utilize hybridization of a probe nucleic acid to nucleic acids corresponding to the genetic marker. Hybridization formats include but are not limited to, solution phase, solid phase, mixed phase or in situ hybridization assays. Markers which are restriction fragment length polymorphisms (RFLP), are detected by hybridizing a probe (which is typically a sub-fragment or a synthetic oligonucleotide corresponding to a sub-fragment of the nucleic acid to be detected) to restriction digested genomic DNA. The restriction enzyme is selected to provide restriction fragments of at least two alternative (or polymorphic) lengths in different individuals and will often vary from line to line. Determining a (one or more) restriction enzyme that produces informative fragments for each cross is a simple procedure, well known in the art. After separation by length in an appropriate matrix (e.g., agarose) and transfer to a membrane (e.g., nitrocellulose, nylon), the labeled probe is hybridized under conditions which result in equilibrium binding of the probe to the target followed by removal of excess probe by washing. Nucleic acid probes to the marker loci can be cloned and/or synthesized. Detectable labels suitable for use with nucleic acid probes include any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radiolabels, enzymes and colorimetric labels. Other labels include ligands which bind to antibodies labeled with fluorophores, chemiluminescent agents and enzymes. Labeling markers is readily achieved such as by the use of labeled PCR primers to marker loci.

The hybridized probe is then detected using, most typically, autoradiography or other similar detection technique (e.g., fluorography, liquid scintillation counter, etc.). Examples of specific hybridization protocols are widely available in the art.

Amplified variable sequences refer to amplified sequences of the plant genome which exhibit high nucleic acid residue variability between members of the same species. All organisms have variable genomic sequences and each organism (with the exception of a clone) has a different set of variable sequences. Once identified, the presence of specific variable sequence can be used to predict phenotypic traits. Preferably, DNA from the plant serves as a template for amplification with primers that flank a variable sequence of DNA. The variable sequence is amplified and then sequenced.

In vitro amplification techniques are well known in the art. Examples of techniques sufficient to direct persons of skill through such in vitro methods, including the polymerase chain reaction (PCR), the ligase chain reaction (LCR), O,β-replicase amplification and other RNA polymerase mediated techniques (e.g., NASBA), are readily found in the art. One of skill will appreciate that essentially any RNA can be converted into a double stranded DNA suitable for restriction digestion, PCR expansion and sequencing using reverse transcriptase and a polymerase.

Oligonucleotides for use as primers, e.g., in amplification reactions and for use as nucleic acid sequence probes, are typically synthesized chemically according to the solid phase phosphoramidite triester method, or can simply be ordered commercially.

Alternatively, self-sustained sequence replication can be used to identify genetic markers. Self-sustained sequence replication refers to a method of nucleic acid amplification using target nucleic acid sequences which are replicated exponentially in vitro under substantially isothermal conditions by using three enzymatic activities involved in retroviral replication: (1) reverse transcriptase, (2) Rnase H and (3) a DNA-dependent RNA polymerase. By mimicking the retroviral strategy of RNA replication by means of cDNA intermediates, this reaction accumulates cDNA and RNA copies of the original target.

As mentioned above, there are many different types of molecular markers, including amplified fragment length polymorphisms (AFLP), allele-specific hybridization (ASH), single nucleotide polymorphisms (SNP), simple sequence repeats (SSR), and isozyme markers. Methods of using the different types of molecular markers are known to those skilled in the art.

The qAG-9-2 QTL and genes LOC_Os09g20390 (SEQ ID NO: 4; OsTPP7); LOC_Os08g20380 (SEQ ID NO: 3); and LOC_Os08g20400 (SEQ ID NO: 5), or homologs thereof, in the genome of a plant exhibiting a preferred phenotypic trait may be determined by any method listed above, e.g., RFLP, AFLP, SSR, etc. If the nucleic acids from the plant are positive for one or more desired genetic markers, the plant can be selfed to create a true breeding line with the same genotype or it can be crossed with a plant with the same marker or with other desired characteristics to create a sexually crossed hybrid generation.

It will be recognized by one skilled in the art that the materials and methods of the present invention may be similarly used to confer improved early vigor during germination, particularly, improved tolerance to anaerobic germination, in plants other than rice, such as corn, wheat, barley, sorghum, millet, oat, rye, sunflower, canola; and soybean

### EXAMPLES

### Example 1

### The Trehalose-6-Phosphate Phosphatase OsTPP7 Confers Tolerance to Anaerobic Conditions During Germination in Rice

Materials and Methods

QTL Confirmation and Finemapping of qAG-9-2

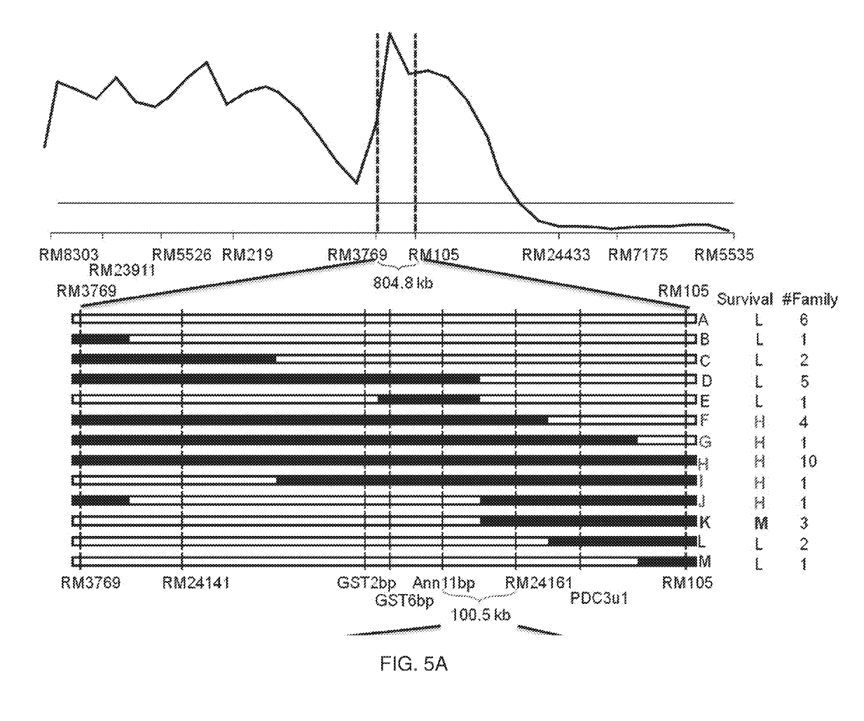

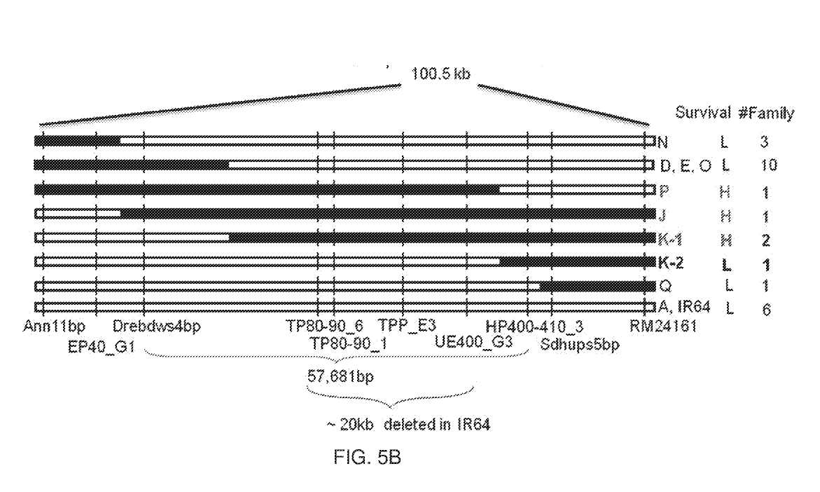

Near isogenic lines (NILs) were developed for the major QTL, qAG-9-2 by backcrossing selected BC₂F₃ progenies that have the QTL target and few non target backgrounds to the recurrent parent IR64, by maintaining small introgressions of KHO DNA fragments in the QTL region, and selecting against the rest of the genome. Seven BC₄F₃ introgression lines developed from two selected BC₂F₃ from the original mapping populations (individuals #101 and 270) having different size of introgression in the QTL region were used to confirm the presence of the QTL. The same seven BC₄F₃ groups but without the QTL qAG-9-2 introgression were used for comparison. These seven families still have several non target backgrounds; however, since qAG-9-2 was a major QTL, it was expected that the effect of the QTL could still be seen. For this experiment, a total of 144 lines from the seven families were used. Following the confirmation, selected BC₄F₄ recombinant families were used to fine map the QTL. In the first round of fine mapping, 260 lines from 38 different NILs with different size of the QTL introgression were used (FIG. 5A). Following the first batch of the fine mapping, genotyping was performed to identify more recombinants from the rest of the BC₄F₄ populations that were developed using the closest flanking markers identified in the first batch of fine mapping. The newly identified recombinants were then phenotyped and further genotyped along with the recombinants identified in the first batch using additional newly developed markers generated inside the closest markers that flanked the QTL. There were additional 50 recombinant lines from 9 different NILs used to further fine map the QTL (FIG. 5B; Table 1)

TABLE 1
Primers Used

| SEQ ID | | | |
|---|---|---|---|
| NO: | Primer name | Primer sequence (5′-3′) | Primer use |
| 23 | GST2bpF | TCACTTTGGTGCCATTTTCA | Fine mapping |
| 24 | GST2bpR | TATGCCGTGGCTTTTAGGAC | Fine mapping |
| 25 | GST6bpF | CATCATCACTGATCGGCAAG | Fine mapping |
| 26 | GST6bpR | GCTACAACCACCATGCACAC | Fine mapping |
| 27 | Ann11bpF | CTCCCCGAGAGGCACTTC | Fine mapping |
| 28 | Ann11bpR | CAACTTGAGCAACTCCACGA | Fine mapping |
| 29 | EP40_G1F | GGCACACTTCGCCTTACCTA | Fine mapping |
| 30 | EP40_G1R | CCATGACTCCATCCCAAAAC | Fine mapping |
| 31 | DrebUps6bpF | ACGGGATTAGCATAGGGTCA | Fine mapping |
| 32 | DrebUps6bpR | GGCGATGAGAGAGAGAGAGC | Fine mapping |
| 33 | Drebdws4bpF | CCGTGCAGATGGGATTTTAG | Fine mapping |
| 34 | TPS_GE1F | GGTGAACCCACCGATTTATG | Fine mapping (monomorphic) |
| 35 | TPS_GE1R | GTTGTTGTGAAGGAGCAGCA | Fine mapping (monomorphic) |
| 36 | TPS_GE2F | CATGACAGACAGGCAGAAGG | Fine mapping (monomorphic) |
| 37 | TPS_GE2R | GGTACAAATGCCTAACGCAAA | Fine mapping (monomorphic) |
| 38 | Tp80-90_7F | CGATGGTCGGATATTCTCGT | Fine mapping (dominant) |
| 39 | Tp80-90_7R | TGTTTAAATCAATCATTGGGAAT | Fine mapping (dominant) |
| 40 | Tp80-90_6F | GTCCGCTGCAAAGGAAATAA | Fine mapping (dominant) |
| 41 | Tp80-90_6R | CGAGTGATAATTTGCATGCTT | Fine mapping (dominant) |
| 42 | Tp80-90_5F | TTCCACACATGATTGGCTACA | Fine mapping (dominant) |
| 43 | Tp80-90_5R | AGAGGCAGCTTTACGCTCAC | Fine mapping (dominant) |
| 44 | Tp80-90_4F | GTAGCTAGGCGCCAAAAGG | Fine mapping (dominant) |
| 45 | Tp80-90_4R | ACGAAAGCATAGGCATCCAC | Fine mapping (dominant) |
| 46 | Tp80-90_3F | AGGGGGTGAGGTTTTGAATC | Fine mapping (dominant) |
| 47 | Tp80-90_3R | AACCACAGCCTACGCATACA | Fine mapping (dominant) |
| 48 | Tp80-90_1F | CCCACCCTCCTTAAATTCCT | Fine mapping (dominant) |
| 49 | Tp80-90_1R | AGGTACTGCACCCGAAGAGA | Fine mapping (dominant) |
| 50 | TPP_E3F | GATCCTGGTGTCCAAGCAC | Fine mapping (dominant) |
| 51 | TPP_E3R | TACAGCCTCAACCGTGTCCT | Fine mapping (dominant) |
| 52 | TPP_G4F | AATGGTGTCCACATTGCAGA | Fine mapping (dominant) |
| 53 | TPP_G4R | GCATTGATCTTCCTCTTGTGC | Fine mapping (dominant) |
| 54 | TPP_D1F | CAACGTAGCAACAGGCTGAA | Fine mapping (dominant) |
| 55 | TPP_D1R | TCGTTGGATCCATTGACAGA | Fine mapping (dominant) |
| 56 | TPP_D2F | CTGTGTGCTCGTTGACCACT | Fine mapping (dominant) |
| 57 | TPP_D2R | GCAGCGTCGTACCTACCTTC | Fine mapping (dominant) |
| 58 | TPP_D3F | ATCGATCCATGGTGCATTTT | Fine mapping (dominant) |
| 59 | TPP_D3R | GCGTGGATTGTAGGCATGTA | Fine mapping (dominant) |
| 60 | TPP_D4F | ACGACTGTCCACAGAAACCA | Fine mapping (dominant) |
| 61 | TPP_D4R | TGGAGAGTGCAAATCGACAA | Fine mapping (dominant) |
| 62 | TPP_D5F | TGGGGATATCATCTGTGCAT | Fine mapping (dominant) |
| 63 | TPP_D5R | CACATCCAACCCCTCATCTT | Fine mapping (dominant) |
| 64 | TPP_D6F | GAATCAAAATTGGACGAGCA | Fine mapping (dominant) |
| 65 | TPP_D6R | TCAGGTCGGTTGTTGCTACT | Fine mapping (dominant) |
| 66 | TPP_D10F | CTGAGCCAATCAATCTTCGAG | Fine mapping (dominant) |
| 67 | TPP_D10R | TGCATGTTGAGTTTTGTGAGC | Fine mapping (dominant) |
| 68 | TPP_D11F | GGGTAGAGAAGCCGAGGAAG | Fine mapping (dominant) |
| 69 | TPP_D11R | CACGCTTTTCAAACTGCTGA | Fine mapping (dominant) |
| 70 | TPP_D12F | GCTCCGGTGCTCTCTACTGT | Fine mapping (dominant) |
| 71 | TPP_D12R | AGGTGCGGAGGATATAATCG | Fine mapping (dominant) |
| 72 | TPP_D13F | AGTGTGAGTGTGGCAAGTGG | Fine mapping (dominant) |
| 73 | TPP_D13R | CTCAGGCCAAAGAGGTTCAG | Fine mapping (dominant) |
| 74 | TPP_D14F | TTGATGGCATTCAACTTTGG | Fine mapping (dominant) |
| 75 | TPP_D14R | ATCCCTAGGTACACGGACCA | Fine mapping (dominant) |
| 76 | TPP_D15F | TAGTCCCCAAACGGTGAAAG | Fine mapping (dominant) |
| 77 | TPP_D15R | GCCACCGAATCATTTTTCAT | Fine mapping (dominant) |
| 78 | TPP_D16F | TTCCTACCATTTTTGGCGTTA | Fine mapping (dominant) |
| 79 | TPP_D16R | CGCTATTTCAGAAGGTAACGAG | Fine mapping (dominant) |
| 80 | TPP_D17F | TTCTGTTGCTGGCTGTCATC | Fine mapping (dominant) |
| 81 | TPP_D17R | GGTCGAGGCAATTATGCAAT | Fine mapping (dominant) |
| 82 | TPP_D18F | GGTAGATCCGCCCCTAAAGA | Fine mapping (dominant) |
| 83 | TPP_D18R | AGGGGTTCCTAACGCCTCTA | Fine mapping (dominant) |
| 84 | TPP_D19F | AACCCCACCTTTGGATTGTT | Fine mapping (dominant) |
| 85 | TPP_D19R | CGTTTTTGTAGGATGCGTCA | Fine mapping (dominant) |
| 86 | TPP_D20F | CACAAAGCCTCAGATCAGGA | Fine mapping (dominant) |
| 87 | TPP_D20R | GCGTAATGCTGCTGCTCA | Fine mapping (dominant) |
| 88 | UE400_G3F | CGTAATGCTGCTGCTCAGG | Fine mapping (dominant) |
| 89 | UE400_G3R | CGGTTCACTTTTGGGAACAT | Fine mapping (dominant) |
| 90 | UE400_G2F | CGTCTTCGTCTTCGAGATGC | Fine mapping (monomorphic) |
| 91 | UE400_G2R | TTTGATACGCATCGCACAAT | Fine mapping (monomorphic) |
| 92 | UE400_G1F | ATTTGCCGTATGGACATGCT | Fine mapping (monomorphic) |
| 93 | UE400_G1R | ACAGCACGAAGAGGCTGAC | Fine mapping (monomorphic) |
| 94 | HP400-410_2F | TGCGACTGTGATTCTGCTCT | Fine mapping |
| 95 | HP400-410_2R | TCATTTCTCTCCTCCACTTCATC | Fine mapping |
| 96 | HP400-410_3F | TCCAGCTCCTTACGGCTTT | Fine mapping |
| 97 | HP400-410_3R | ATATCGGTCGACAGCGAGAC | Fine mapping |
| 98 | Sdhups5bpF | GATGCACTCCCTCTGTTGCT | Fine mapping |
| 99 | Sdhups5bpR | TCACCTTCTTACCGAACACCA | Fine mapping |
| 100 | PDC3u1F | GTTCAACGTGGTTGCACAAT | Fine mapping |
| 101 | PDC3u1R | GATGCAAGCTTGGTCGTGT | Fine mapping |
| 102 | TPP_F | GCCCTAGGGTTCTTGACTGGAAGGTTTC | Cloning |
| | | TTTG | |
| 103 | TPP_R | GCGGTACCTTCAATTGTTACAGCCTCAA | Cloning |
| | | CC | |
| 104 | prUbi-F | GCAAGCTTCGGTCGTGCCCCTCTCTA | Cloning |
| 105 | prUbi-R | GCCCTAGGTCTAGAGTCGACCTGCAGA | Cloning |
| | | AG | |
| 106 | TPP_P_Hind_F | AAGCTTGGTGTATAACCGTTGTTCCGTG | Cloning |
| | | AGC | |
| 107 | TPP_P_Bam_R | CCTAGGCACCAAAGAAACCTTCCAGTC | Cloning |
| | | AAGAAC | |
| 108 | TPP_P_R_AvrII_ | CCTAGGCACCAAAGAAACCTTCCAGTC | Cloning |
| | R | AAGAAC | |
| 109 | FLEX_SV1_F | GATGGCGATCGCCATGGCGAAGGCGAG | Cloning |
| | | CGTGGTG | |
| 110 | FLEX_SV1_R | CTAAGTTTAAACCAGCCTCAACCGTGTC | Cloning |
| | | CTGGACAG | |
| 111 | DFR_F2 | CCACCATGATGTAGTTCAGTTGTGAAC | DEL Marker |
| 112 | DFR_R2 | CACCGTTAAAATCGGCCGTTAG | DEL Marker |
| 113 | TDNA-LB1 | TAGTTCCCAGATAAGGGAATTAGGGTT | TAIL PCR |
| 114 | TDNA-LB2_ | GGTTTCGCTCATGTGTTGAGCATATAA | TAIL PCR |
| 115 | TDNA-LB3 | CAGTACTAAAATCCAGATCCCCCGAAT | TAIL PCR |
| 116 | TDNA-LB4_ | ACGTCCGCAATGTGTTATTAAGTTGTC | TAIL PCR/homozygosity |
| 117 | AD1 | NTCGASTWTSGWGTT | TAIL PCR |
| 118 | AD2 | NGTCGASWGANAWGAA | TAIL PCR |
| 119 | AD3 | WGTGNAGWANCANAGA | TAIL PCR |
| 120 | pfg_tpp_F | GCTGCTGCTACACGTAGTCG | KO homozygosity |
| 121 | pfg_tpp_R | GACACTGACATGAACCGTGC | KO homozygosity |
| 122 | TLBP2 | TCCTCTAGAGTCGAGAATTCAGTAC | KO homozygosity |
| 123 | HPT_F | AGCTGCA-FCATCGAAATTGCCG-FC | T-DNA insertion test |
| 124 | HPT_R | TGTTTATCGGCACTTTGCATCGGC | T-DNA insertion test |
| 125 | NP1_HOMO_F | TCCACGACCACAAGGCAAAC | NP1 homozygosity |
| 126 | NP1_HOMO_F | TGCAATCGACCAGCAGCAG | NP1 homozygosity |
| 127 | NP2_HOMO_F | AGCTTACCGATGGGCACCAC | NP2 homozygosity |
| 128 | NP2_HOMO_F | TCGCAGGGGAAATTATCAGG | NP2 homozygosity |
| 129 | qTPP_L | GGGAGGATGGTGTTCGAG | qPCR |
| 130 | qTPP_R | AGCGAGTCGAGGAGGAACT | qPCR |
| 131 | qPOLU_L | CAGCAGCGCCTCATCTTC | qPCR |
| 132 | qPOLU_R | GGATGTTGTAGTCAGCCAAGG | qPCR |
| 133 | qUBI_L | CTCAAGGACCTGCAGAAGGA | qPCR |
| 134 | qUBI_R | ATGGACCCATCAGTGTTGC | qPCR |
| 135 | qATU_L | TGTTGATTATGGAAAGAAGTCCAA | qPCR |
| 136 | qATU_R | GAGGACACTGTTGTATGGTTCTACA | qPCR |
| 137 | TPP_RT_F | AACAAGGGAGTCCTCTTCCAG | Semi-qPCR |
| 138 | TPP_RT_R | CTTGAACGCGTCCTCGTC | Semi-qPCR |
| 139 | AG1_360_RT_F | CCACTGGACAAGGAGGTAGG | Semi-qPCR |
| 140 | AG1_360_RT_F | TCAACTCCTCTCCCACGAGGATTCG | Semi-qPCR |
| 141 | AG1_370_RT_F | TTTTAGCAGTACTCCGACTGC | Semi-qPCR |
| 142 | AG1_370_RT_R | TTCTAAAGCGGGTGGTGG | Semi-qPCR |
| 143 | AG1_400_RT_F | AGCAAGTCGGTCGTGTCC | Semi-qPCR |
| 144 | AG1_400_RT_R | GACCCTGAGCAGCAGCAT | Semi-qPCR |
| 145 | DFR_F0 | ATGAAGACCATGTGTCACTGTCAC | DEL Marker |
| 146 | DFR_F1 | AATTCTGCTACTACCAACTCCAGAG | DEL Marker |
| 147 | DFR_F2 | CCACCATGATGTAGTTCAGTTGTGAAC | DEL Marker |
| 148 | DFR_R0 | ATTGGCTTCGAAAGTGAGTGCAC | DEL Marker |
| 149 | DFR_R2 | CACCGTTAAAATCGGCCGTTAG | DEL Marker |
| 150 | DFR_R3 | AATTAGGAGCAAAATCACGCAAAACTG | DEL Marker |
| 151 | DFR_LB1 | TCGATGGCCTCCAGAAGGTC | DEL Marker |
| 152 | DFR_LB2 | CGGCTTCGTCTTCACCTGAAC | DEL Marker |
| 153 | DFR_RB1 | GGCGTTCCCTCCTTCTTATGG | DEL Marker |
| 154 | DFR_RB2 | GCATTGGGGCAGTGTTGTTGTATG | DEL Marker |

Screening under AG stress was conducted following a known protocol. The screening was performed using grid trays, wherein each tray can accommodate 11 entries consisting of 9 samples and 2 parental controls (IR64 and KHO), with 30 seeds used per entry. Dry seeds were put on trays which were already filled with 1.5 cm of fine soil. Once a tray was filled with seeds, another 1 cm layer of fine soil was put on top of the seeds to cover them. For the QTL confirmation, 16 trays per replication were used to accommodate 144 BC₄F₃ introgression lines and the two parental controls in each tray, thus a total of 176 entries were used per replication. The whole set up per rep was put on a raised concrete bench and carefully submerged in 9-10 cm water. Two replications were used and survival rate was scored 21 days after sowing. Randomization for all entries including the parental controls was performed with Alpha Plus design. A seed germination test was also performed under normal condition using 30 seeds per entry to check the seed vigor using a petridish with a sheet of moist filter paper in an incubator at 30° C. Germination rate was counted after 7 days. Phenotypic screening for fine mapping was also conducted for the first and the second sets of fine mapping with a similar set up as used for the QTL confirmation.

Whole Genome Re-Sequencing and RNA Sequencing Analysis

Genomic DNA of KHO and IR64 was extracted using the DNeasy-Plant Maxi kit (Qiagen) according to the manufacturer's instructions. RNA of four day old coleoptiles and embryos was extracted using the RNeasy-Plant Mini kit (Qiagen) according to the manufacturer's instructions. RNA quality and integrity was checked on a 2100 Bioanalyzer (Agilent) according to the manufacturer's instructions. Whole genome sequencing and cDNA-based whole transcriptome sequencing (RNAseq) was performed by a sequencing service provider (Macrogen) on an Illumina Hiseq2000 platform generating 100 bp paired-end reads with an average insert size of 300 bp. The generated Fastq files were processed and analyzed using the software suite CLC-Genomics-Workbench 7 (Qiagen). Trimmed reads were mapped against the Nipponbare reference MSU6.1 and MSU7.

Cloning, Transformation, and Genotyping for Mutant Plants

Fragment encompassing the full-length coding region of TPP7 gene (LOC_Os09g20390; SEQ ID NO: 4) was amplified from genomic DNA (rice cv. Khao Hlan On) using KAPA Hifi DNA Polymerase Hotstart (Kapa Biosystems, Woburn, Mass.) with oligonucleotides TPP_F (SEQ ID NO: 102) and TPP_R (SEQ ID NO: 103). The oligonucleotide pair introduced AvrII and KpnI restriction sites to the amplified fragment at its 5′ and 3′ end, respectively. The oligonucleotides prUbi-F (SEQ ID NO: 104) and prUbi-R (SEQ ID NO: 105) were used to amplify a 1986 bp fragment of the maize polyubiquitin promoter from pCAMBIA1300int::prUbi::tNOS. The oligonucleotide pair introduced HindIII and AvrII restriction sites to the amplified fragment at its 5′ and 3′, respectively. The oligonucleotides TPP_P_Hind_F (SEQ ID NO: 106) and TPP_P_AvrII_R (SEQ ID NO: 108) were used to amplify a 1927 bp fragment of the TPP7 promoter from genomic DNA (rice cv. Khao Hlan On). The oligonucleotides TPP_P_Hind_F (SEQ ID NO: 106) and TPP_P_Bam_R (SEQ ID NO: 107) were used to amplify a 1927 bp fragment of the TPP7 promoter from genomic DNA (rice cv. Khao Hlan On).

The fragments were introduced to the pCR4Blunt-TOPO vector following the manufacturer's instructions (Invitrogen, Carlsbad, Calif.).

The overexpression construct pCAMBIA1300int::prUbi::TPP::tNOS was assembled by ligating the maize polyubiquitin promoter (HindIII-AvrII fragment) and the TPP gene (AvrII-KpnI fragment) in between the HindIII and KpnI sites of pCAMBIAint::tNOS. The native promoter construct pCAMBIA1300int::prTPP::TPP::tNOS was assembled by replacement of the prUBI fragment with the TPP_P promoter fragment using HindIII and AvrII restriction sites. The pCAMBIA1300::prTPP::GUS construct was assembled by inserting the TPP_P promoter fragment into pCAMBIA1300int::GluA2p::GUS::tNOS using HindIII and BamHI restriction sites.

Agrobacterium-mediated transformation of the overexpression construct and native promoter construct into susceptible parent (rice cv. IR64) was performed using immature embryo following the procedure established by Hiei and Komari (2006). The GUS construct was transformed into tolerant parent (Khao Hlan On) using calli derived from mature seeds following the protocol described by Toki (2006). The Agrobacterium strain LBA4404 was employed. Regenerated transgenic plantlets (T0) were transferred to the greenhouse and grown in hydroponic culture. After three weeks, the plants were transplanted into soil. Plants were grown under a natural light condition in the greenhouse where temperature was controlled between 24 and 30° C.

T1 transformants were tested for successful T-DNA integration via PCR using HPT_F and HPT_R primers that amplify a 600 pb fragment of the hygromycin resistance gene of the pCAMBIA1300 series.

In the segregating T1 generation homozygous mutant plants and their corresponding wild type plants were identified either via germination trials on hygromycin containing plates or via TAIL PCRs.

For TPP overexpression lines germination tests on hygromycin-containing plates (75 μM) were performed. Lines were considered homozygous for an insertion if more than 90% of a batch of >30 seeds germinated and grew unrestricted. Lines were considered homozygous null segregants if less than 5% of a batch of >30 seeds germinated and grew unrestricted. T1 generation lines were further more checked for segregation of the antibiotic resistance trait. Four lines were confirmed homozygous and tested phenotypically. Detailed studies were performed on two independent lines of the T2 generation.

A total of six independent TPP native promoter (NP) lines were generated and TAIL PCRs were performed on the T1-derived gDNA using primers of SEQ ID NOs: 113-119 in order to determine the T-DNA insertion sites and allow for design of T-DNA flanking primers to determine homozygosity. For four lines, homozygous insertion mutants and homozygous null segregant lines were identified and tested phenotypically. Detailed studies were performed on two independent lines of the T2 generation. Neither of the two lines contained the T-DNA insertion in a genic region.

For pOsTPP::GUS plants, more than 100 lines were generated, of which more than 50 lines were positively tested for GUS staining in germinating tissues of the T1 generation. Five independent lines in the T2 generation were used for detailed studies.

A T-DNA insertion mutant, containing an insertion in the third exon of LOC_Os09g20390 (SEQ ID NO: 4) was identified in the Dongjin background via in-silico screening of SIGNAL DB. CLON PFG_3A-08739.L was then obtained from the Crop Developmental Biology Lab. Seeds (T1) were germinated and the resulting lines checked for homozygosity with a three primer approach, using the T-DNA insertion flanking primers pfg_tpp_F (SEQ ID NO: 120) and pfg_tpp_R (SEQ ID NO: 121) in combination with the T_DNA left border specific primer TLBP2 (SEQ ID NO: 122). Homozygous mutant lines and homozygous null segregants were carried into the T2 generation and used for phenotypic analysis.

RNA Extraction, Semiquantitative RT-PCR, and Quantitative RT-PCR

Embryos and coleoptiles were dissected from seeds using a scalpel.

RNA extraction and clean up was performed using the RNeasy kit (Qiagen) with in-column DNAse digest according to the manufacturer's instructions. Reverse transcription was performed using the GoScript Reverse transcription system (Promega) according to the manufacturer's instructions.

Semiquatitative RT PCR was performed for LOC_Os09g20360 (primers of SEQ ID NOs: 139-140), LOC_Os09g20370 (primers of SEQ ID NOs: 141-142:), LOC_Os09g20390 (primers of SEQ ID NOs: 137-138), and LOC_Os09g20400 (primers of SEQ ID NOs: 143-144), with α-tubulin serving as housekeeping control.

Quantitative RT PCR was performed using a Roche system and Roche consumables.

Primers qTPP_L (SEQ ID NO: 129) and qTPP_R (SEQ ID NO: 130) were used for LOC_Os09g20390 (SEQ ID NO: 4; OsTPP7). α-tubulin (LOC_Os07g38730.1) using primers qATU_L (SEQ ID NO: 135) and qATU_R (SEQ ID NO: 136), Polyubiquitin (LOC_Os06g46770.1) using primers qPOLU_L (SEQ ID NO: 131) and qPOLU_R (SEQ ID NO: 132), ubiquitin (LOC_Os02g16040.1) using primers qUBI_L (SEQ ID NO: 133) and qUBI_R (SEQ ID NO: 144), were used as reference genes. The REST software (Qiagen) was used for calculation of expression differences and statistical analysis. Four biological replicates were used for analysis.

Plant Growth Conditions

Since seed age and storage reportedly affects AG tolerance, seeds for all analysis were used in a period between 3 months and 18 months after harvest. All seeds compared within an experiment were of equal age. Seeds were stored at 4° C. after post-harvest processing. Seed dormancy was broken by incubation at 37° C. for 5 days. For expression analysis, coleoptile elongation studies and amylase assays, seeds were de-hulled, sterilized in 70% ethanol for 2 min, washed three times with sterile water and submerged in 7 cm of autoclaved distilled water for 1-4 days at 30° C. in the dark. A minimum of three biological replicates containing a minimum of 69 individual coleoptiles were used for analysis. For sucrose complementation experiments, water was substituted with sterile 90 mM sucrose solution. Three biological replicates containing a minimum of 46 individual coleoptiles were used for analysis.

α-Amylase Assays

Amylase activity was quantified as previously described. Around 20 seeds were ground in liquid nitrogen and extracted twice with 1.5 ml A buffer (0.006 M NaCl, 0.02 M NaH₂PO₄/Na₂HPO₄, pH 6.9). Crude extracts were quantified for protein content using Sigma (Sigma Aldrich). Amylase activity of crude extract was measured by end point quantification of soluble starch (0.5 mg/ml in A buffer) breakdown stopped after 3 min by incubation at 95° C. for 5 min in equal volume of DNS solution. OD was measured and converted using a maltose standard curve. For total amylolytic enzyme activity crude extract was used, while for α-amylase activity crude extract was incubated at 70° C. for 15 min A minimum of three biological replicates were used for analysis.

Quantitative Metabolite Analysis

Quantification of sucrose, glucose-6-phosphate, T6Phosphate and trehalose was performed on the basis of GC_MS analysis by a commercial service provider (MetabolomicDiscoveries, Germany)

TPP Enzyme Activity Assays

The TPP coding sequence (LOC_Os09g20390.1; SEQ ID NO: 7) was amplified from coleoptile cDNA using the oligonucleotides FLEX_SV13 (SEQ ID NO: 109) and FLEX_SV1_F (SEQ ID NO: 110) and subsequently cloned into the pFLEXI bacterial expression system (Promega) according to the manual.

The recombinant TPP7 was purified according to the manufacturer's instructions.

TPP enzyme activity was monitored by quantification of released phosphate from T6P via spectrophotometry, using the BIOMOL Green Reagent (Enzo Life Science) according to the manufacturer's instructions.

Results

Fine-Mapping of the Kho Hlan on QTL qAG-9-2

The Burmese landrace Khao Hlan On (KHO) is a known donor of tolerance of anaerobic germination (AG). Bi-parental mapping analysis with IR64 as a susceptible recipient led to the identification of qAG-9-2, a large effect quantitative trait locus (QTL) for AG.

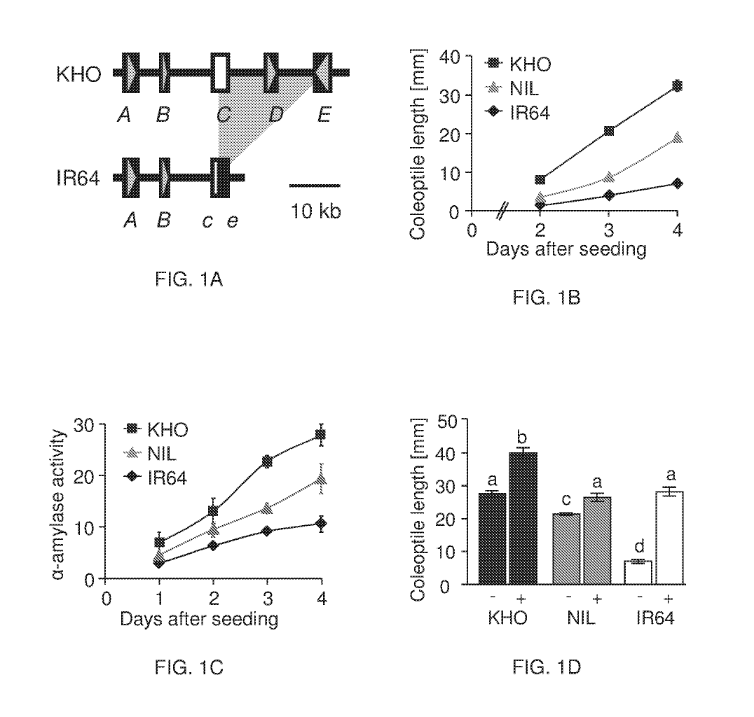

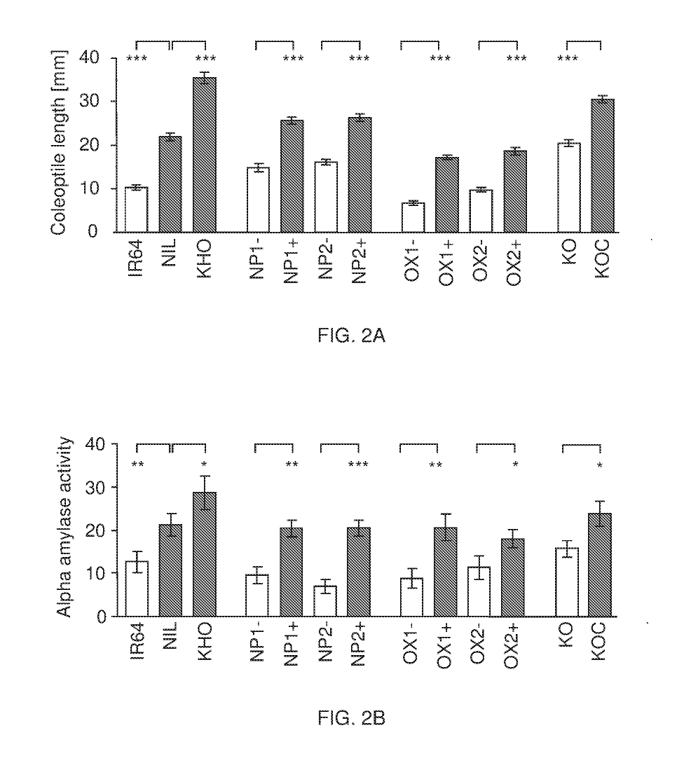

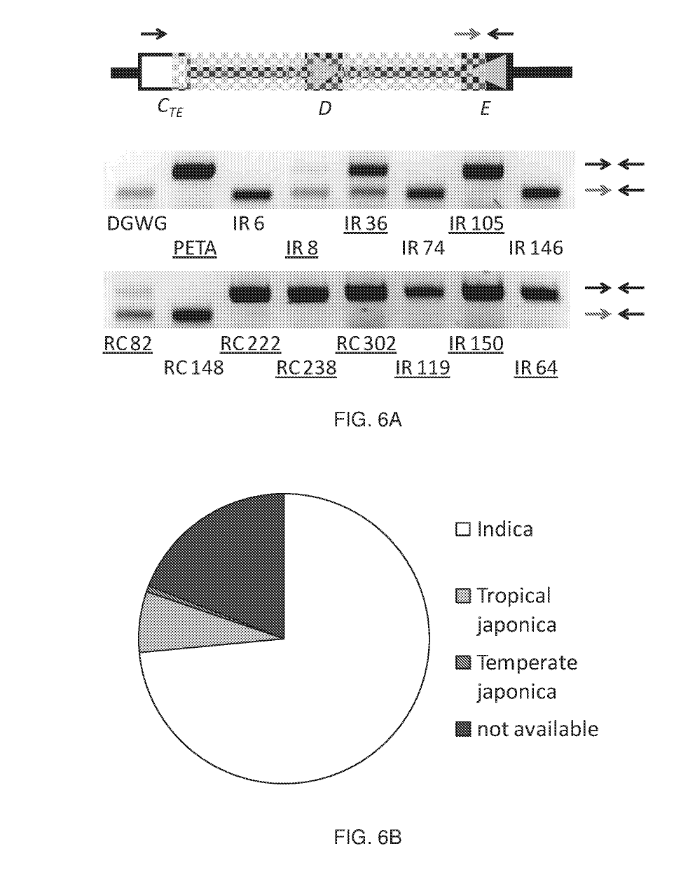

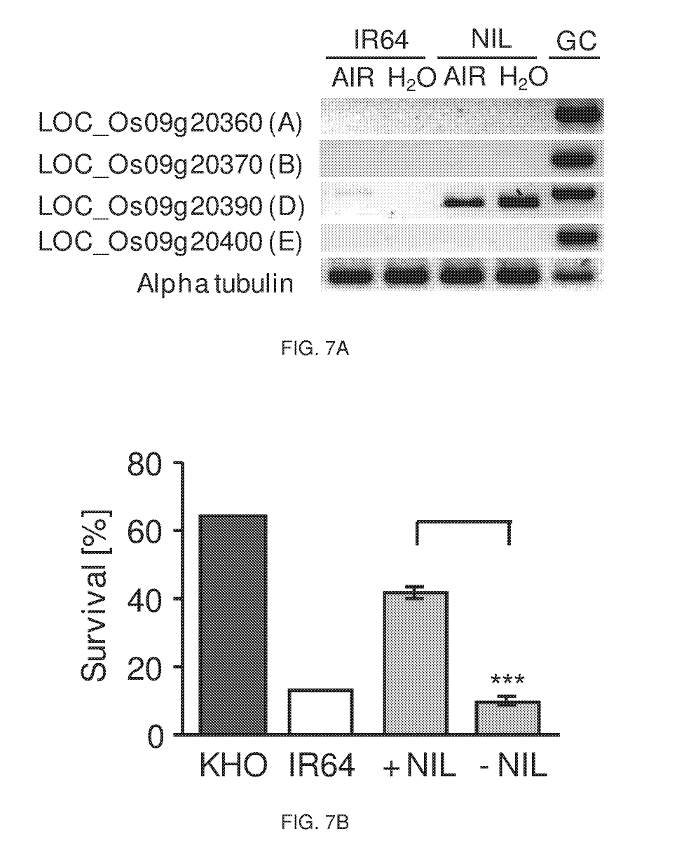

The presence of qAG-9-2 in KHO was confirmed in the present study. The QTL was fine-mapped to a region of less than 60 kb at 12.25 Mb on Chromosome 9 (FIG. 1A, FIG. 5). The qAG-9-2 region contained four gene loci and one transposable element in the reference genome (KHO; FIG. 1A). In IR64, several markers across qAG-9-2 failed to amplify (FIG. 5B; Table 1), demonstrating a deletion. Cloning and sequencing of the INDEL region showed a 20.9 kb deletion (FIG. 1A, FIG. 5B) encompassing LOC_Os09g20390 (SEQ ID NO: 4; OsTPP7; Os09g0369400) and causing truncation of the neighboring loci (FIG. 1A). This deletion was prevalent in International Rice Research Institute (IRRI)-derived cultivars (FIG. 6A), tracing back to the green revolution variety IR8. In silico screening of a diversity panel of 780 accessions identified the deletion in around 18% of accessions. Approximately 75% of these belonged to the Indica group and approximately 7.5% belonged to the Japonica group (FIG. 6B) demonstrating the deletion to be Indica-derived. Of the four genes within the qAG-9-2 region only LOC_Os09g20390 (SEQ ID NO: 4; OsTPP7) was expressed at detectable levels in germinating tissues of a qAG-9-2-containing near isogenic line (NIL) (FIG. 5, FIG. 7A), while, consistent with the deletion, expression was absent in IR64 (FIG. 7A). qAG-9-2 NILs in the IR64 background displayed enhanced levels of AG tolerance as monitored by increased levels of survival under submergence (FIG. 7B).

Identification of Improved Starch Mobilization as the Mechanism of qAG-9-2-Mediated AG Tolerance

Morphologically, AG tolerance is characterized by rapid and extensive coleoptile elongation under submergence, with concomitant delay of epicotyl and radicle development. Investing resources into coleoptile growth under submergence serves as an escape strategy, eventually enabling allocation of surface oxygen towards the developing embryo. Developmental restrictions associated with AG are largely linked to abolishment of respiratory ATP regeneration. Consequently, efficient utilization of starch reserves, has been reported as pivotal to attenuate energy starvation symptoms and maintain growth under AG.

KHO and NIL66 displayed enhanced coleoptile elongation as compared to IR64 (FIG. 1b). Coleoptile lengths differed significantly between parents and NIL after two to four days of growth in the dark under submergence (DOGS). In agreement with this finding, α-amylase activities showed significant differences after one to four DOGS between IR64, NIL66, and KHO (FIG. 1C). AG-susceptibility of IR64 was rescuable by supplementation with sugar. When grown in sucrose solution, IR64 increased coleoptile lengths by 4.1 fold, to sizes similar to NIL66 coleoptiles in sucrose (FIG. 1D). This demonstrated that carbon availability is a key limiting factor for IR64 during AG.

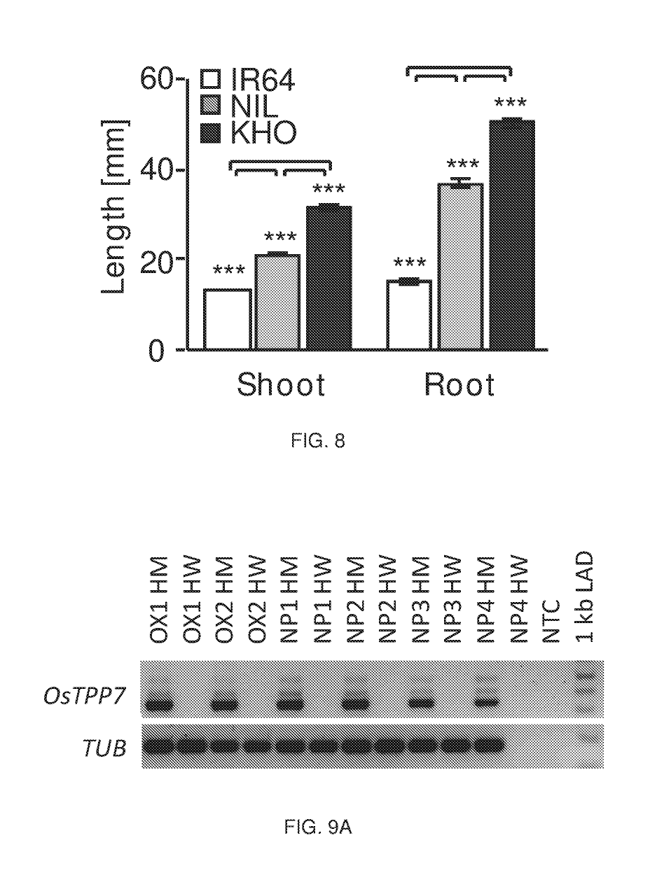

Early growth under aerobic conditions was then tested to determine whether rapid starch mobilization affected early vigor in general. KHO and NIL66 outperformed IR64 in terms of root and shoot elongation after four days of growth in the dark (FIG. 8), demonstrating that qAG-9-2 had growth promoting effects independent of the oxygen environment.

Overall, the NIL performed intermediately as compared to KHO and IR64 Improved reserve starch mobilization to sustain vigorous growth was the predominant mechanism of qAG-9-2-mediated AG tolerance.

Identification of OsTPP7 as the Causal Gene Underlying qAG-9-2

Absence of LOC_Os09g20390 (SEQ ID NO: 4; OsTPP7; Os09g0369400) in IR64 and its expression in the NIL under AG demonstrated it to be the causal gene underlying qAG-9-2. Os09g0369400 is annotated as a trehalose-6-phosphate phosphatase (OsTPP7).

Rice contains 13 TPP-like genes, of which OsTPP1 and OsTPP2 have been partially characterized and demonstrated to convert trehalose-6-phosphate (T6P) to trehalose. T6P is known to act as an indicator of sugar availability in sink organs and was found indispensable for growth and development in Arabidopsis. High T6P concentrations signal ample sugar supply, activating starch synthesis, and inhibiting SnRK1 (a sucrose non-fermenting-1-related protein kinase 1), while T6P amounts rapidly deplete under carbon starvation, releasing SnRK1 from inhibition. SnRK1 is a central integrator of sugar and energy related signals to coordinate a starvation response in plants. High SnRK1 activity in response to energy/sugar depletion leads to a shift from anabolism to catabolism in order to maintain energy homeostasis. In rice, SnRK1 plays a key role during germination and early seedling growth via induction of α-amylase expression Importance of SnRK1-related signaling in relation to AG tolerance in rice has been demonstrated by characterization of CIPK15 (calicineurin B-like-interacting protein kinase 15), which directly modulates SnRK1 activity under oxygen limitation, activating starch breakdown and fermentation.

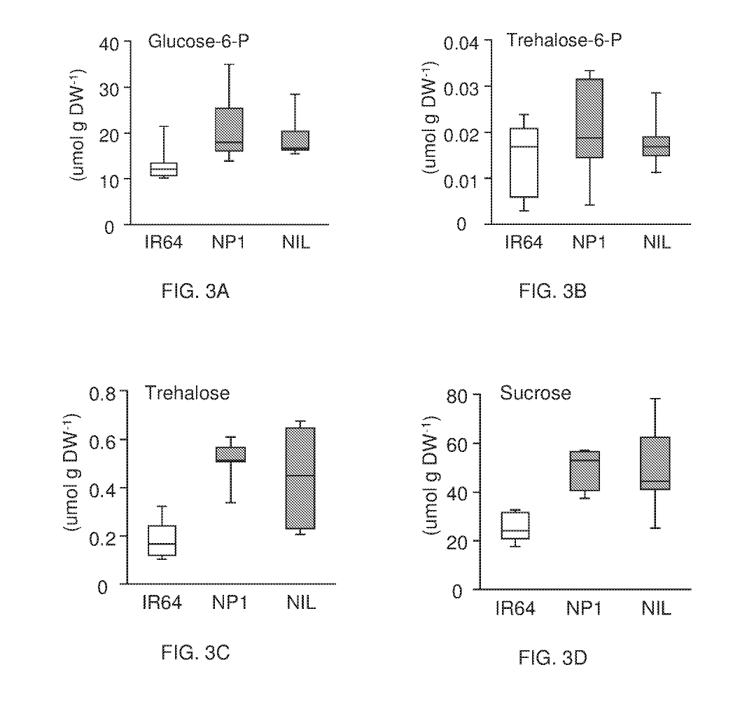

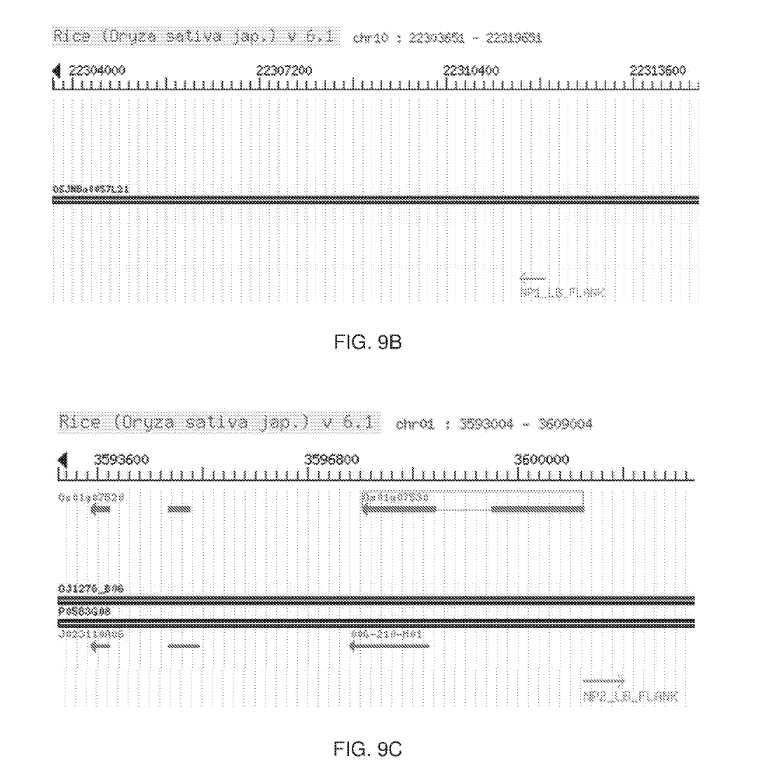

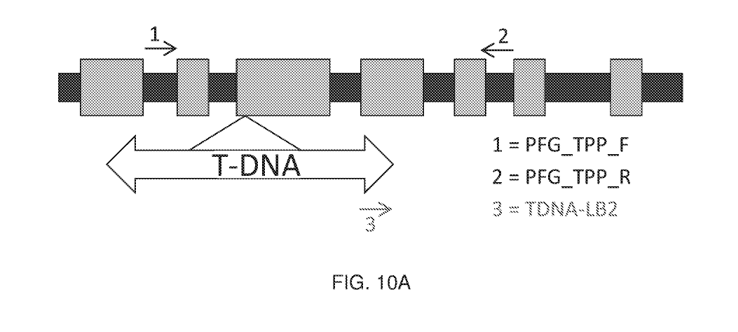

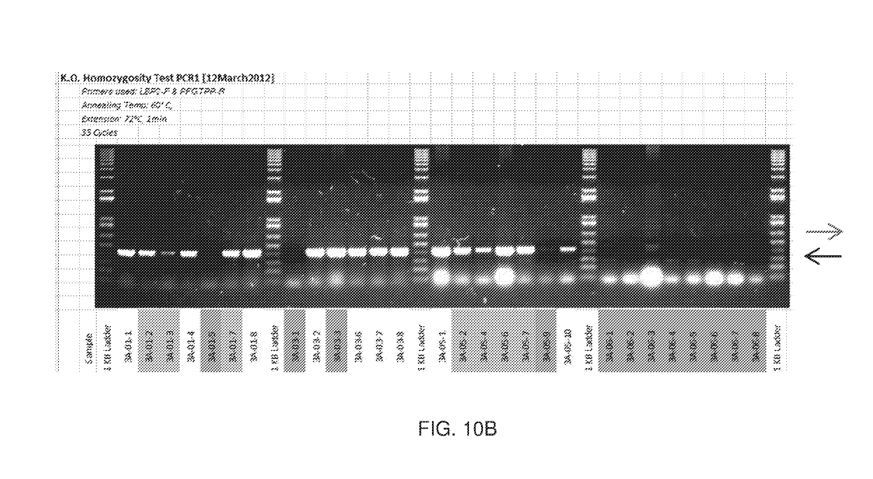

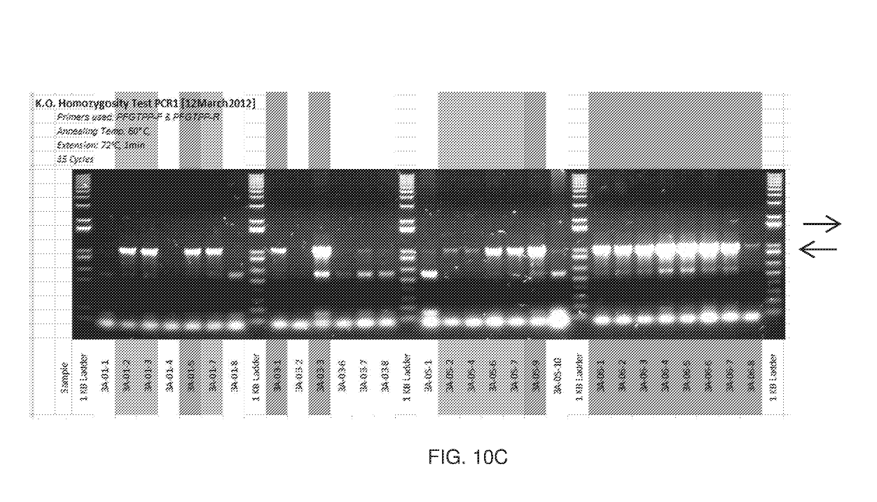

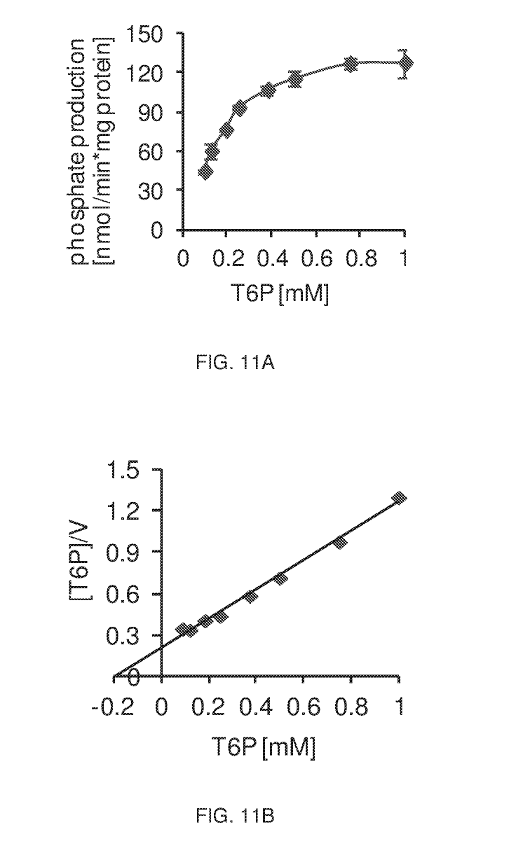

Transgenic IR64 lines carrying the KHO allele of OsTPP7 under the control of either its native promoter (NP) or the constitutive maize ubiquitin promoter (OX) (FIG. 9). Presence of a functional OsTPP7 allele correlated with qAG-9-2-mediated AG tolerance in all investigated lines as monitored by coleoptile length (FIG. 3A) and α-amylase activity (FIG. 3B) after 4 DOGS. Coleoptiles were 2.1 fold longer for NIL66 than for IR64. Two independent NP lines displayed 1.6-1.7 fold longer coleoptiles than their respective null segregants, while two independent OX lines displayed 1.9-2.6 fold longer coleoptiles. Coleoptiles of the KO line were 1.5 fold shorter than those of the null segregant. Amylase activities were 1.7 fold higher for qAG-9-2-NIL than for IR64. NP lines showed 2.1-2.9 and OX lines showed 1.6-2.3 fold higher amylase activities than their respective null segregants. Amylase activities of the KO line were on average 1.5 fold lower than those of the null segregant.

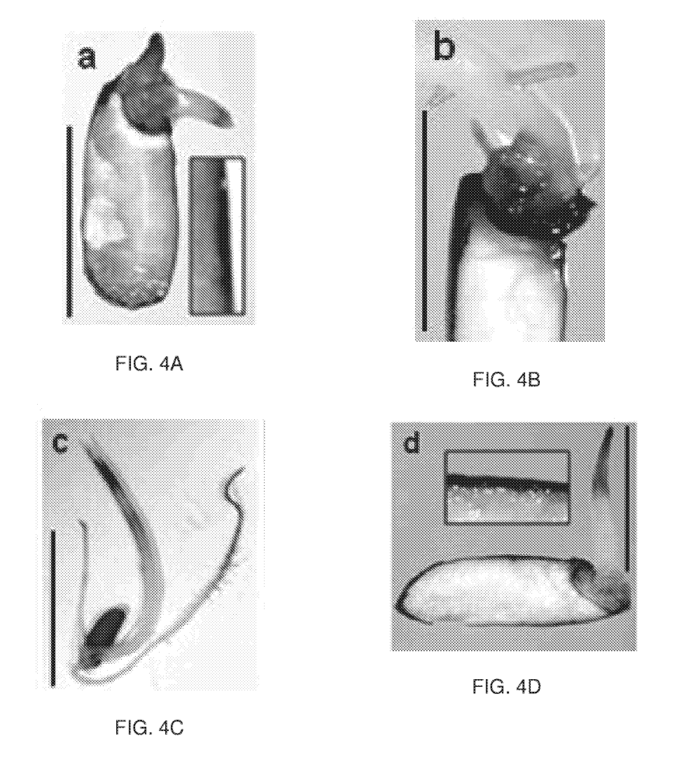

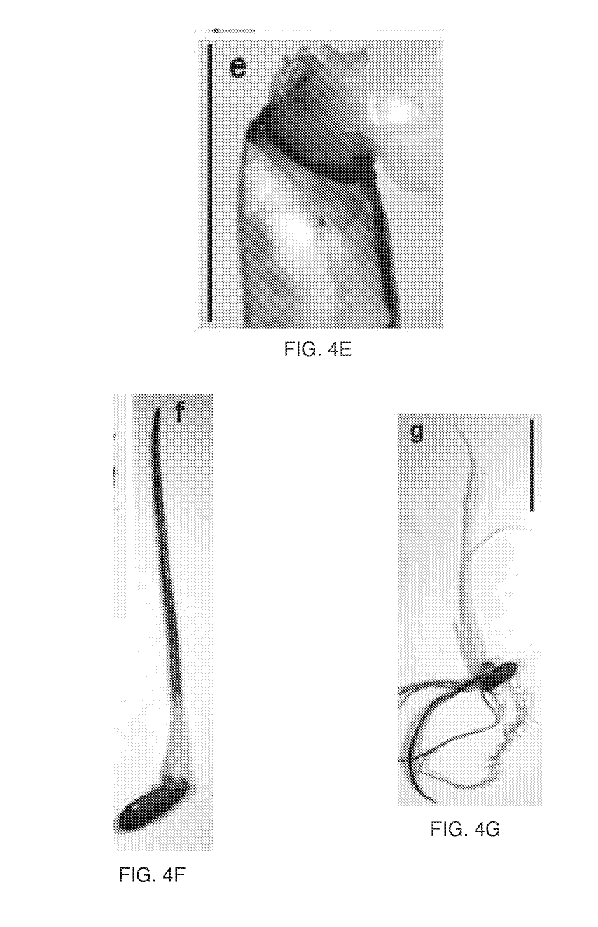

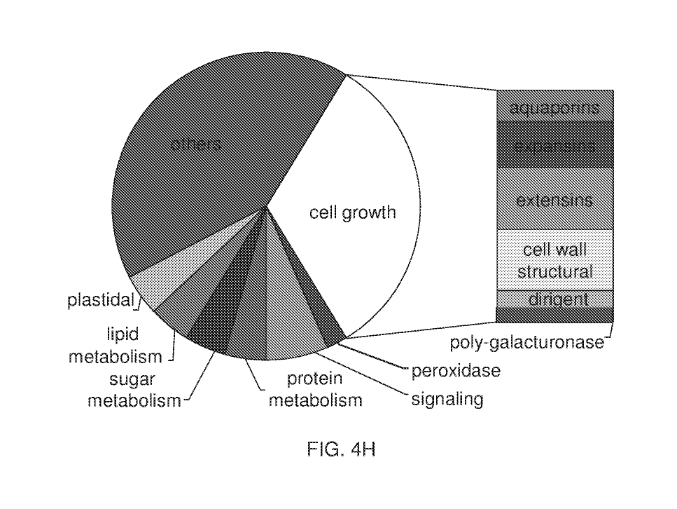

To investigate whether presence of OsTPP7 affects the concentrations of sugars that are linked to T6P metabolism, glucose-6-P, T6P, trehalose and sucrose were quantified in freeze-dried tissues of embryos and coleoptiles of IR64, NP1 and the NIL after 4 DOGS (FIGS. 4A-4D).

Function of OsTPP7 In Vivo

T6P is generated from UDP-glucose and glucose 6-phosphate by trehalose 6-phosphate synthase (TPS) and converted to trehalose by TPP. T6P is a general indicator of sucrose availability.

Presence or absence of OsTPP7 in the IR64 background did not have significant effects on glucose-6-phosphate or T6P concentration (FIGS. 4A-4B). Trehalose concentrations were on average 2.7 fold higher in NP1 and 2.3 fold higher in the NIL (FIG. 4C), indicating that OsTPP7 catalyzes the conversion of T6P to trehalose in vivo. Sucrose concentrations were, on average, 1.9 fold higher in NP1 and 2.0 fold higher in the NIL (FIG. 4C), showing that OsTPP7 activity results in higher sucrose availability.

Though T6P concentrations were comparable in both backgrounds, the relative amounts of T6P to sucrose were reduced in the OsTPP7-containing lines, indicating a change in T6P-sucrose homeostasis. T6P synthesis rates largely depend on substrate concentrations, making T6P an indicator of sugar levels. Therefore, increased sucrose translates into increased T6P. Local T6P concentrations, however, depend on both rates of synthesis and rates of turnover. Higher concentrations of trehalose in OsTPP7-containing lines indicate enhanced T6P turnover. Consequently, OsTPP7 acts as an enhancer of sink strength, maintaining increased sucrose allocation to growing tissues by preventing concomitant increase of the sucrose-availability signal T6P, which would otherwise dampen sink strength through feedback inhibition.

Recombinant OsTPP7

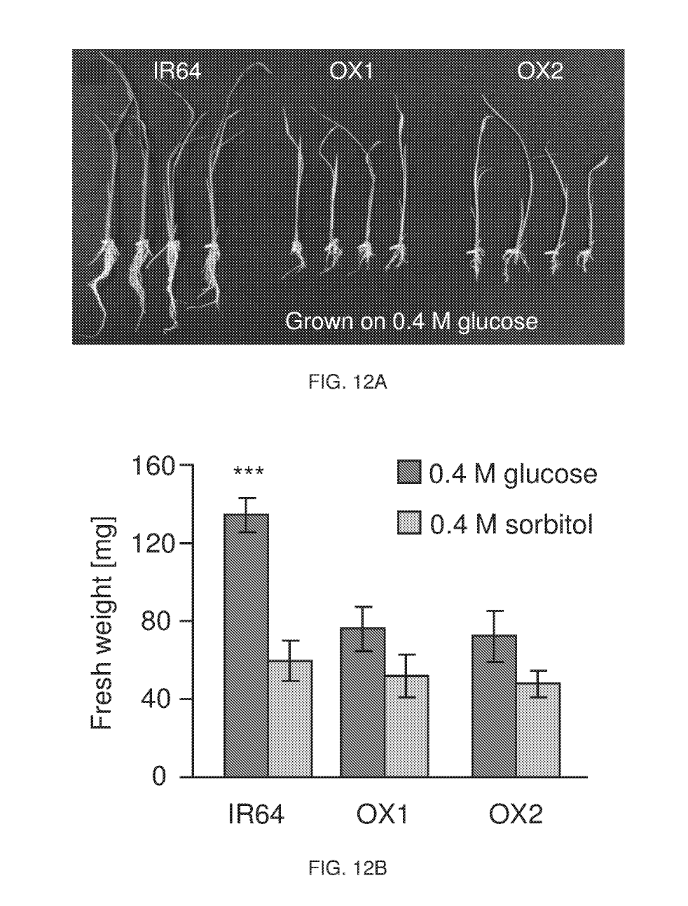

OsTPP7 activity in vivo was supported by the observation that recombinant OsTPP7 was able to dephosphorylate T6P in vitro (FIG. 8). Recombinant TPP displayed a high affinity for T6P (apparent Km=0.2 mM). Additional evidence for OsTPP7 functionality came from the finding that OX lines were hypersensitive to external glucose applications (FIG. 12), a phenotype previously described for Arabidopsis lines over-expressing the bacterial TPP otsB, which was attributed to a global deregulation of sugar signaling.

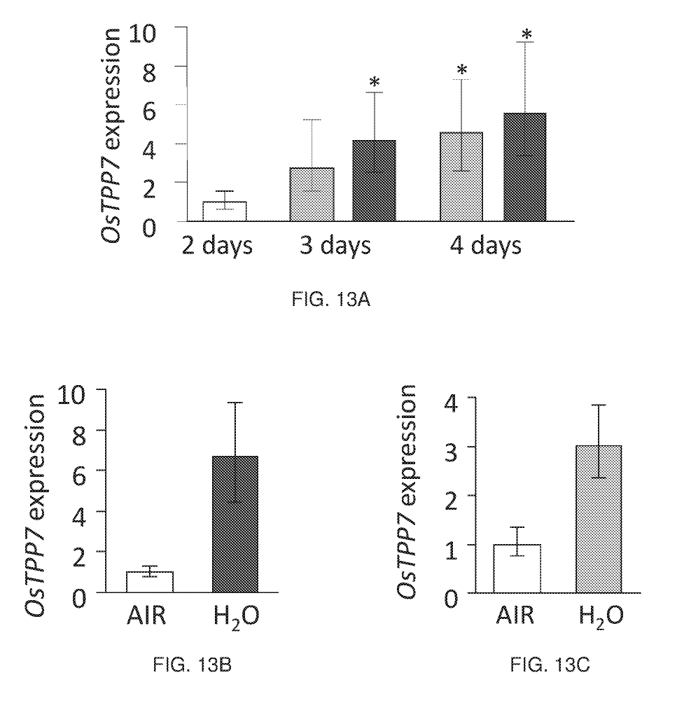

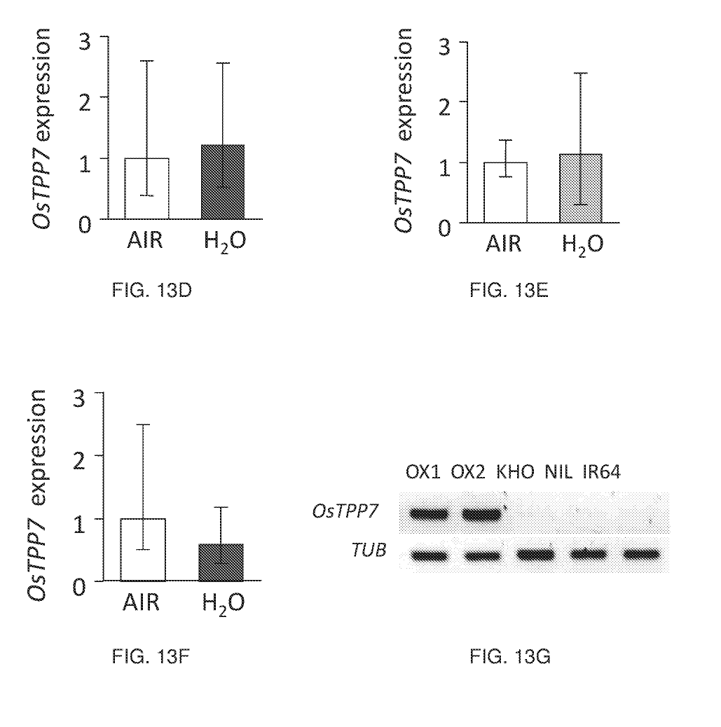

OsTPP7 promoter-GUS (pOsTPP7::GUS) studies showed OsTPP7 expression in embryo, coleoptile, roots, the aleuron layer, and the scutellar epithelium (FIGS. 4A-4F). Embryonic pOsTPP7::GUS signal appeared higher in aerated samples (FIGS. 4A-4C), whereas signal in coleoptiles appeared higher in submerged samples (FIGS. 4D-4F). Grown under 16 h daylight cycling for 6 days, pOsTPP7::GUS expression was absent in leaves, but present in roots (FIG. 4G). PCR-based expression analysis supported the pOsTPP7::GUS data, showing a clear increase for OsTPP7 expression between two and four days DOGS and absence of OsTPP7 expression in leaves (FIG. 13).

Collectively, OsTPP7 was expressed in young heterotrophic tissues independent of submergence, which is in line with the finding that qAG-9-2-dependent early vigor phenotypes were independent of the oxygen environment (FIG. 8). T6P is a negative signal of sink strength and congruently OsTPP7 expression was found in young sink tissues that depend on sugar allocation from source tissues, while it was absent in autotrophic source tissues (FIG. 4G).

Activation of α-Amylases by OsTPP7-Catalyzed T6P Turnover

High expression of OsTPP7 was apparent in the scutellar region separating embryo from endosperm (FIGS. 4A, 4B, 4D, and 4E). The scutellar epithelium expresses and secretes α-amylases into the starchy endosperm. Expression of scutellar α-amylases is sugar inhibited via SnRK1-dependent signaling, T6P being the responsible signal. After imbibitions, rapid depletion of sugars in the embryo initially induces α-amylase expression, but subsequent sugar release and transfer from the starchy endosperm results in α-amylase repression.

The observed activation of α-amylases through OsTPP7-catalyzed T6P turnover is relayed through SnRK1-dependent signaling pathways via alleviation of T6P-mediated SNRK1 inhibition. Thus, OsTPP7 acts as an upstream modulator of SnRK1 that fine tunes local T6P concentrations as input signals of local sucrose status.

Effects of OsTPP7 on Global Gene Expression

In order to investigate whether OsTPP7 had effects on global gene expression, total transcript amounts were compared between pooled embryos and coleoptiles of IR64 and NP1 lines after 4 DOGS via RNA sequencing analysis. A total of 62 genes were found to be differentially expressed with a minimum fold change of 1.5 (Tables 2 and 3). Of the 46 up-regulated genes, more than one third were related to cell growth (FIG. 4B), reflecting investments into coleoptile elongation due to improved carbon status.

TABLE 2
Genes significantly upregulated by at least 1.5 fold, with false discovery rate
corrected p-values below 0.05 in an OsTPP7-containing IR64 (NP1) vs. IR64

| | EDGE test: | EDGE test: | EDGE test: | | |
|---|---|---|---|---|---|
| | WT14 vs | WT14 vs | WT14 vs | | |
| | AG1_MU14 | AG1_MU14 | AG1_MU14 | | |
| | tagwise | tagwise | tagwise | | |
| | dispersions- | dispersions- | dispersions- | | |
| | Fold | P- | FDR p-value | Annotations-Annotation | |
| Feature ID | change | value | correction | notes | Ontology |
| LOC_Os04g02910_2 | 2.84 | 6.294E−06 | 1.064E−02 | | other |
| LOC_Os11g06720_1 | 2.42 | 1.476E−05 | 2.171E−02 | abscisic stress-ripening; | signaling |
| | | | | putative; expressed | |
| LOC_Os03g31679_1 | 1.62 | 2.781E−05 | 3.345E−02 | annexin A7; putative; | other |
| | | | | expressed | |
| LOC_Os02g44630_1 | 2.49 | 3.028E−08 | 1.252E−04 | aquaporin protein; putative; | cell growth |
| | | | | expressed | |
| LOC_Os04g16450_1 | 1.86 | 1.390E−05 | 2.090E−02 | aquaporin protein; putative; | cell growth |
| | | | | expressed | |
| LOC_Os06g37560_1 | 2.46 | 2.330E−05 | 2.908E−02 | beta-galactosidase | sugar |
| | | | | precursor; putative; metabolism | |
| | | | | expressed | |
| LOC_Os09g17740_1 | 9.01 | 2.421E−10 | 2.002E−06 | chlorophyll A-B binding | early |
| | | | | protein; putative; expressed | photosynthesis |
| LOC_Os04g53190_1 | 2.09 | 6.432E−06 | 1.064E−02 | CPuORF12-conserved | other |
| | | | | peptide uORF-containing | |
| | | | | transcript; expressed | |
| LOC_Os08g09010_1 | 17.54 | 4.832E−08 | 1.682E−04 | Cupin domain containing | other |
| | | | | protein; expressed | |
| LOC_Os03g59440_1 | 6.69 | 1.927E−05 | 2.601E−02 | dirigent; putative; expressed | ROS-related |
| LOC_Os01g14410_1 | 5.08 | 7.498E−09 | 3.543E−05 | early light-induced protein; | early |
| | | | | chloroplast precursor; | photosynthesis |
| | | | | putative; expressed | |
| LOC_Os10g40720_1 | 2.57 | 3.565E−13 | 4.760E−09 | expansin precursor; | cell growth |
| | | | | putative; expressed | |
| LOC_Os02g44108_1 | 1.92 | 1.692E−05 | 2.402E−02 | expansin precursor; | cell growth |
| | | | | putative; expressed | |
| LOC_Os01g60770_1 | 1.91 | 3.363E−07 | 9.672E−04 | expansin precursor; | cell growth |
| | | | | putative; expressed | |
| LOC_Os07g39450_1 | 26.57 | 3.063E−05 | 3.516E−02 | expressed protein | other |
| LOC_Os07g05840_1 | 7.86 | 4.690E−07 | 1.293E−03 | expressed protein | other |
| LOC_Os05g36210_1 | 6.34 | 4.362E−06 | 7.799E−03 | expressed protein | other |
| LOC_Os09g10274_1 | 2.98 | 6.678E−06 | 1.077E−02 | expressed protein | other |
| LOC_Os05g03590_1 | 2.81 | 1.766E−06 | 3.633E−03 | expressed protein | other |
| LOC_Os01g45720_1 | 2.28 | 7.825E−07 | 1.917E−03 | expressed protein | other |
| LOC_Os07g03120_1 | 1.82 | 9.135E−06 | 1.439E−02 | expressed protein | other |
| LOC_Os01g11730_1 | 12.96 | 4.905E−11 | 4.636E−07 | GDSL-like | lipid |
| | | | | lipase/acylhydrolase; | metabolism |
| | | | | putative; expressed | |
| LOC_Os01g27390_1 | 8.26 | 9.865E−07 | 2.331E−03 | glutathione S-transferase; | ROS-related |
| | | | | putative; expressed | |
| LOC_Os10g31640_1 | 3.30 | 5.561E−09 | 3.344E−05 | glycine-rich cell wall | cell growth |
| | | | | structural protein 2 | |
| | | | | precursor; putative; | |
| | | | | expressed | |
| LOC_Os10g31720_1 | 3.20 | 2.569E−07 | 7.725E−04 | glycine-rich cell wall | cell growth |
| | | | | structural protein 2 | |
| | | | | precursor; putative; | |
| | | | | expressed | |
| LOC_Os10g31710_1 | 2.75 | 8.168E−09 | 3.602E−05 | glycine-rich cell wall | cell growth |
| | | | | structural protein 2 | |
| | | | | precursor; putative; | |
| | | | | expressed | |
| LOC_Os10g31660_1 | 2.62 | 1.177E−06 | 2.513E−03 | glycine-rich cell wall | cell growth |
| | | | | structural protein 2 | |
| | | | | precursor; putative; | |
| | | | | expressed | |
| LOC_Os04g46830_1 | 2.44 | 1.897E−11 | 2.091E−07 | LTPL122-Protease | protein |
| | | | | inhibitor/seed storage/LTP | metabolism |
| | | | | family protein | precursor; |
| | | | | expressed | |
| LOC_Os10g40510_1 | 2.09 | 4.265E−05 | 4.478E−02 | LTPL144-Protease | protein |
| | | | | inhibitor/seed storage/LTP | metabolism |
| | | | | family protein precursor; | |
| | | | | expressed | |
| LOC_Os11g38810_1 | 2.15 | 1.269E−05 | 1.952E−02 | mannose-6-phosphate | sugar |
| | | | | isomerase; putative; | metabolism |
| | | | | expressed | |
| LOC_Os06g35970_1 | 1.83 | 4.742E−06 | 8.254E−03 | meiosis 5; putative; | other |
| | | | | expressed | |
| LOC_Os01g74300_1 | 3.11 | 7.266E−09 | 3.543E−05 | metallothionein; putative; | thionin |
| | | | | expressed | |
| LOC_Os12g38010_1 | 1.72 | 1.783E−05 | 2.457E−02 | metallothionein; putative; | thionin |
| | | | | expressed | |
| LOC_Os03g18070_1 | 1.92 | 2.928E−05 | 3.459E−02 | omega-3 fatty acid | lipid |
| | | | | desaturase; chloroplast | metabolism |
| | | | | precursor; putative; | |
| | | | | expressed | |
| LOC_Os02g56380_1 | 3.81 | 4.778E−08 | 1.682E−04 | OsWAK21-OsWAK | signaling |
| | | | | receptor-like cytoplasmic | |
| | | | | kinase OsWAK-RLCK; | |
| | | | | expressed | |
| LOC_Os03g25330_1 | 4.79 | 2.229E−05 | 2.836E−02 | peroxidase precursor; | ROS-related |
| | | | | putative; expressed | |
| LOC_Os10g05970_1 | 3.54 | 7.270E−09 | 3.543E−05 | POEI12-Pollen Ole e I | cell growth |
| | | | | allergen and extensin family | |
| | | | | protein precursor; expressed | |
| LOC_Os10g05980_1 | 4.73 | 2.527E−05 | 3.095E−02 | POEI13-Pollen Ole e I | cell growth |
| | | | | allergen and extensin family | |
| | | | | protein precursor; expressed | |
| LOC_Os10g05990_1 | 2.98 | 4.154E−08 | 1.617E−04 | POEI14-Pollen Ole e I | cell growth |
| | | | | allergen and extensin family | |
| | | | | protein precursor; expressed | |
| LOC_Os10g06000_1 | 2.58 | 1.104E−06 | 2.434E−03 | POEI15-Pollen Ole e I | cell growth |
| | | | | allergen and extensin family | |
| | | | | protein precursor; expressed | |
| LOC_Os05g50260_1 | 3.27 | 3.598E−13 | 4.760E−09 | polygalacturonase; putative; | cell growth |
| | | | | expressed | |
| LOC_Os04g33390_1 | 2.18 | 1.812E−06 | 3.633E−03 | prephenate dehydratase | protein |
| | | | | domain containing protein; | metabolism |
| | | | | expressed | |
| LOC_Os10g31670_1 | 2.19 | 3.382E−05 | 3.728E−02 | retrotransposon protein; | other |
| | | | | putative; unclassified; | |
| | | | | expressed | |
| LOC_Os07g24830_1 | 2.84 | 1.033E−06 | 2.356E−03 | thionin-like peptide; | thionin |
| | | | | putative; expressed | |
| LOC_Os07g25050_1 | 2.63 | 2.053E−06 | 3.994E−03 | thionin-like peptide; | thionin |
| | | | | putative; expressed | |
| LOC_Os01g08380_1 | 4.30 | 4.239E−05 | 4.478E−02 | transferase family protein; | other |
| | | | | putative; expressed | |
| LOC_Os09g20390_1 | 155.61 | 3.730E−20 | 1.234E−15 | uncharacterized glycosyl | TRANSGENE |
| | | | | hydrolase Rv2006/MT2062; | |
| | | | | putative; expressed | |

TABLE 3
Genes significantly downregulated by at least 1.5 fold, with false discovery rate
corrected p-values below 0.05 in an OsTPP7-containing IR64 (NP1) vs. IR64

| | EDGE test: | EDGE test: | EDGE test: | | |
|---|---|---|---|---|---|
| | WT14 vs | WT14 vs | WT14 vs | | |
| | AG1_MU14 | AG1_MU14 | AG1_MU14 | | |
| | tagwise | tagwise | tagwise | | |
| | dispersions- | dispersions- | dispersions- | | |
| | Fold | P- | FDR p-value | Annotations-Annotation | |
| Feature ID | change | value | correction | notes | Ontology |
| LOC_Os01g03360_1 | −1.53 | 3.083E−05 | 3.516E−02 | BBTI5-Bowman-Birk type | other |
| | | | | bran trypsin inhibitor | |
| | | | | precursor; expressed | |
| LOC_Os05g33140_1 | −8.48 | 2.200E−07 | 6.931E−04 | CHIT5-Chitinase family | other |
| | | | | protein precursor; expressed | |
| LOC_Os07g36630_1 | −1.91 | 5.036E−07 | 1.333E−03 | CSLF8-cellulose synthase- | cell growth |
| | | | | like family F; beta1;3;1;4 | |
| | | | | glucan synthase; expressed | |
| LOC_Os11g10590_1 | −1.70 | 2.033E−05 | 2.689E−02 | expressed protein | other |
| LOC_Os03g02470_3 | −10.00 | 3.472E−36 | 2.297E−31 | expressed protein | other |
| LOC_Os06g04930_1 | −18.27 | 2.109E−05 | 2.736E−02 | expressed protein | other |
| LOC_Os04g46810_1 | −3.81 | 3.235E−13 | 4.760E−09 | LTPL120-Protease | other |
| | | | | inhibitor/seed storage/LTP | |
| | | | | family protein precursor; | |
| | | | | expressed | |
| LOC_Os10g40614_1 | −1.54 | 3.613E−06 | 6.640E−03 | LTPL147-Protease | other |
| | | | | inhibitor/seed storage/LTP | |
| | | | | family protein precursor; | |
| | | | | expressed | |
| LOC_Os06g49190_1 | −2.50 | 4.967E−09 | 3.286E−05 | LTPL154-Protease | other |
| | | | | inhibitor/seed storage/LTP | |
| | | | | family protein precursor; | |
| | | | | expressed | |
| LOC_Os11g02389_1 | −1.55 | 3.381E−05 | 3.728E−02 | protease inhibitor/seed | other |
| | | | | storage/LTP family; | |
| | | | | putative; expressed | |
| LOC_Os03g63074_2 | −3.00 | 3.449E−05 | 3.740E−02 | Ser/Thr protein phosphatase | signaling |
| | | | | family protein; expressed | |
| LOC_Os06g32350_1 | −2.90 | 1.531E−07 | 5.064E−04 | THION12-Plant thionin | other |
| | | | | family protein precursor | |
| LOC_Os06g31890_1 | −1.85 | 3.097E−09 | 2.277E−05 | THION3-Plant thionin | other |
| | | | | family protein precursor; | |
| | | | | expressed | |
| LOC_Os06g32020_1 | −1.78 | 3.061E−06 | 5.786E−03 | THION6-Plant thionin | other |
| | | | | family protein precursor; | |
| | | | | expressed | |
| LOC_Os06g32240_1 | −1.71 | 7.341E−07 | 1.868E−03 | THION9-Plant thionin | other |
| | | | | family protein precursor; | |
| | | | | expressed | |

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. For example in addition to being applicable to rice, the materials and methods disclosed herein may also be applied to other plants, including but not limited to corn, wheat, barley, sorghum, millet, oats, rye, sunflower, and soybean.

Therefore, it is intended that the invention not be limited to the particular embodiment disclosed herein contemplated for carrying out this invention, but that the invention will include all embodiments falling within the scope of the claims.

1-11. (canceled)

12. A method for selecting a plant having improved early vigor during germination relative to a control plant, comprising:
a) inducing expression or increasing expression in a plant, a polynucleotide sharing at least 70% identity with a polynucleotide selected from the group consisting of: SEQ ID NO: 4 (OsTPPT); SEQ ID NO: 7 (OsTPPT); SEQ ID NO:8 (OsTPPT); and SEQ ID NO: 9 (OsTPPT), wherein the induced or increased expression of the polynucleotide is obtained by transforming and expressing in the plant the polynucleotide; and
b) selecting a plant having improved early vigor during germination relative to a control plant.

13. The method of claim 12, wherein the selected plant is further selected for having tolerance to anaerobic germination compared to a control plant.

14. The method of claim 12, wherein the selected plant is further selected for having tolerance of anaerobic germination compared to a control plant, wherein anaerobic germination occurs in conditions of complete submergence.

15. The method of claim 12, wherein the induced or increased expression of the polynucleotide is under the control of at least one promoter functional in plants and wherein the at least one promoter and the polynucleotide are operably linked.

16-17. (canceled)

18. The method of claim 12, wherein the plant is selected from the group of plants consisting of: rice; corn; wheat; barley; sorghum; millet; oats; rye; sunflower; canola; and soybean.

19-52. (canceled)

53. A method of improving plant tolerance to anaerobic germination, the method comprising introducing into a rice plant, being susceptible to anaerobic germination (AG), a nucleic acid sequence that encodes a functional trehalose-6-phosphate phosphatase (TPP), thereby improving tolerance of the plant to anaerobic germination.

54. The method of claim 53, wherein susceptibility to said AG is with respect to the rice cultivar Khao Hlan On.

55. The method of claim 53, wherein said plant comprises a deletion in TPP.

56. The method of claim 53, wherein said plant comprises a deletion in qAG-9-2.

57. The method of claim 55, wherein said deletion in said qAG-9-2 comprises LOC_Os08g20380 (SEQ ID NO: 3), all of LOC_Os08g20390 (SEQ ID NO: 4; OsTPPT), and parts of LOC_Os08g20400 (SEQ ID NO: 5).

58. The method of claim 53, further comprising seeding the plant by direct seeding.

59. The method of claim 58, wherein said rice plant is of the Indica rice group.

60. The method of claim 53, wherein said introducing is by transforming the rice plant with a nucleic acid construct comprising a polynucleotide sharing at least 70% identity with a polynucleotide selected from the group consisting of: SEQ ID NO: 4 (OsTPPT); SEQ ID NO: 7 (OsTPPT); SEQ ID NO:8 (OsTPPT); and SEQ ID NO: 9 (OsTPPT).

61. The method of claim 53, wherein the plant is a rice plant of rice variety IR64 or IR8 or progeny of same.

62. A plant or part thereof having a deletion in endogenous TPP and comprising an exogenous nucleic acid sequence encoding a functional TPP, said plant exhibiting tolerance to AG.

63. The plant of claim 62, being a transgenic plant.

64. The plant of claim 62, being a non-transgenic plant.

65. The plant of claim 62, being of the Indica rice group.

66. The plant of claim 62, wherein the plant is of rice variety IR64 or IR8 or progeny of same.

67. The method of claim 12, wherein the plant having improved early vigor during germination relative to a control plant is selected by detecting presence of the polynucleotide in the transformed plant.


SEQUENCE LISTING (166 sequences)

SEQ ID NO: 1 - DNA, 984 bp, Oryza sativa
atggcgtgggcatcggcggcctggggagaaaggaagatggcggtagcggtggaagaggaggcggtgaaggaagaggcgacatcatttataggtcctaaccttgttctcgttcccgtttcgtaccgtcaacctcgtcgtcatcctccgctccaagccaccttggccagtccgcagcagcagcttgacgatggcctcctcgagtcctccgcctcccacgcttaccactagatccgtgccgtcccattctgtgccaccccgctccatgccgctgagcggccgtcccgctcgtagtctctgtctctccgagctccacgccggcgcgccagccagggtaggatggacgccggtcgtaggtcaggggaggagtcctcggtcgccggtagggggaggagtcaggacattagccggtgaaggaggggagggagccaaagtcgctggctcgttgtcgggagtgagtggaggagaggtcgagaggagataaggggggagagagacgggggagggaagaatttataattttgttttggagtctagggtctttggtataaattttgaacctctacacaaaatgaaaaaagtatgagaactaaatcaaataagtggtagggactgaaatataaatttaaagccacacctaaagccacttgcaatatggagaactcaaattttggacctgtgaccctagcctatatggcagtctgaggctcctcgtttgactgggacacaatgtgaatggctttagtagtttacatcttttgattcttttagttaaaaacttattttatatatagagctatgagaaaacttacaccttctgtgctatgagaaactaacttctgaggattaatctggacaaatgtttatctaaatttatcttaaaaatagttttttttaactattctaggaatcaacgttttttcacggtgcaaaggtaatgataccgagatgtcttggtgtcaatatcgccaataacgttagagttga

SEQ ID NO: 2 - DNA, 1243 bp, Oryza sativa
atggcggggaagaggggcgaagcggcggggcggctgcggaggccggtgaccacggaggcaagggaggcgcggtcggcagcaaggccgaggcagctcggggtcgcgggtgagcgccgtcggcgggtgaggcagctcgaggtcgcgggtgagcagcaaagagaagaggagacgagtggttcaccgtcgccattgaggtcactgttcggcactgaggcggaagcggtggcgctgctaatgctcccttccctggagacggcggtgaggctcccccctctctctctctctcctccttctccactcaggcaggcggtgagcgacaaatcctcctcggggaacgcatggcgcgcgcggtgggcagggcgaggtgggcggcaccgaggacattggaggcggcaggctatcgtgacgagtttgccgcccatccccgtcgtccccgtgcccgattgttcctctgccgcctccactccaatctcctctccttcccgatgtcctcgccggcggctctaccccactggtcccgtcacgacgtgccctccgcaatcttgccgtgcaccagagggaagaaagagagaagagagattttagcagtactccgactgcagccgcatcctctgcgtgctcgctgcgccgcccttgtctccacctccatgccgctgccggcacgtgccgccctcgcttccacctccttgccgctgccggtagtgccgctctactgtgtcgctccgtctccccctgccgccgtgcgctttagaaggaggaagaagagcgccgcgtcgtgccgtctccccaaccaccacccgctttagaaggggatggagaagagagaggtggtggggattctaacgagtgggaactagcgatttttttttaaaaaaactgatgattggactgtcacgtgtgctatttgaatcctaaaccgcttgcaacagtgagcgatatctggttttcaagtttagttggtaattcggtcgatcgcgatagattaggggggtaattcgtactttcctttatattatggattgagaacaagaggatagcttattcttcaacgcatccttgtgacaatctcctgcactcccagataaacagatcatgcatgagcatatatatcgatcttgttagaagatatagctagctttgattgtcagtcaaagatatggctccctttttatcaccatcacagtcaggggcaacatggctagaggtcacttcactatcaacggtcatggatcgagtatactga

SEQ ID NO: 3 - DNA, 3590 bp, Oryza sativa
tcactttttctttgtctcctcccttttctttaactctgatttcttactgggttgcgtatttctcttccttggctcagcttttctcttccgaattgttgccctgccctctttggttgctgtcgttattggcggctgcactgcagcctttcggtttgtggcaataaaatttgattccggcagtggaccaaggtcaactctagaagagttgtttataatgttctgcagaaagtacattgaaattgtgaaaactgttttataaagctataaaatggaaaattggcaaagcattgtctaacctctctcgtcaaggaagacaatatggtattttgtaactgatcatagctaggttggttggttaaaatgctagtctgactgttcagaacttcaggcatgacctgcagaggcataaaatcttcattaccatgttgttctgaaggagcagcactggcttgctgctttcctgcttttatatcttccttcctcttcttgcagcccttcttgttgtgattaactccttgacagtagctacaatgcatttgcacaccatgcttggatatttttgttcctccttcaagttcatgtggctgctttcttctacatctcctaggccttccaactttcttctcataaatcggtgggttcacctttggtccattcatcttcacccaagatgccttgtccctaacaggcattatgttaaatccataggcctccatgtatttttcagttgaataatagaaggataccacatcttcaggttttgtcctctcactcctcaagcaagaaattgcatggttgcatggtatccctgtcaattgccatctcctacattcatagtgttttgccatgaggtcaacaatatattgatgttctttatcagttacttggaacacccctttaccagcaggtaatatatagcatgtatttgacaattctacatttttatcaacctttttctgtatttttatcaacctttttctgtatttttgggcatatgtttcccgaccacttctttacacactcctcttgtttggtatgtatcctattcatgatttggctcctaatcttttcaagcatggataaaattggaagctctctggcttcaagaatgtacttgttgaatacctcggaattgttgttgagtagaatatcacacttggggaaatcactaaagaatgctctgcaccactggttgggtggtatttcctccaggtaggcataagcatctttgctaagagacttcatcttttccatgttaaattcccactctggtatagtacttgaccttgcaattgcccacaactgattctttagagtctcccctttgtatagaacatgaaaattttggtacaaatgcctaacgcaaaatctttgttctgaatctgagaatttatccctcacagctggtaccaatccctacaaaatttaaaaaggaagggtgagtaccaatcmattgaacagtaaataatttcaaatatwttwaaaatgacaagttgaacaaaatcaaatatgatggaataaagtcataccttctgcctgtctgtcatgatagtataaggtccagtgttgtcgataaggagatcttctttcaaagtattcaaaaaccaaccccaactagctctactctccacctccacaacagccatggctataggatagatgcagtcattaggattaatgcctactgcagtcaacaaatgtcccccaaacttggttttgatgtggcatccatcaaggaatatgataggtctacaaccacttaggaatcctcttttgcaagcatcaaatgacacatagcatgtatggaagcacatattttgtaggttcaagaaaaatttactacctggatttgaagtcctaagctcttgtccataatcccacaacatatcgtattgtgctatttcatcaccatatatcgctttcatagctagtttccttgctcgacctaatttatgtcttgatggtgttacatttagttccttttgaactaccttagagaaactcttgagtgtcatcctatcattatccctaaagccttcaacatatctatttgcaagatacctggcagttacataattcaactcccactccttctgacatttgtggccatctatatatctttttaccataagacaattcgtcctgctgtcctcagctacatccaacatccatgagcactcttctttacatttagcctcaatwtttttcttggtatttcttgtaaatttaattgcgactctattcttcacactgtattgttttattgcttgcctaacttcaatagcatcagagaacaacatcccaaccttaaagattggtgactccatgtccacagcagccacaaagttcttaaatttcaacttaatgccttcattatcttcatcagatgactctggaagcaacaagccttcatcatcagagtcatgttcaggaagcttaacaaccatcttgctgctagccacttcttctatctcaacacagttgtcaaaaacatcatcatcccccttgtcagcctcatactcactgtcaacaaagtcaggatctacatcgatgtcatcgtcatcgtcaatgtcatgaccttctataggtttaacatctgaatcttttcgattcattgtagacctctcacggctagccacgataggtatagaagcaggggtagaaagagcaacatcatcccaattgatggattcaagaatgtcttggtgatccacatacaaaagcaatgtcctaaaccttggtgctaatacaaccatgctgttggtgcttgcatcagagttaattctccttagcccatcactgaatgtcttcccaggcaataaccaataaaccttggccttctcagacatatcatatcctagttgctcaataaaatcatcaagccataatgaagaccatgtgtcactgtcacaataatcaaaccaatccaccttttcatccaaataaattctgctactaccaactccagagaaaaaaccaccatgatgtagttcagttgtgaactgttcatcctcgcagcctacatcataagacaaaccattctgatgtgtcaaatcgtaaataattatctgatctattatacacaaaaagattatacagagtacatattcatgcttagagcaaaaattatttctaatcacaaactattcgtgaaggattacttatatgacacctagccttataaaacaccaaagcagtaggcggaggcagcgccgggccaccacaggcaattgcccaggctccatgcatacaacaacactgccccaatgcatataacaacatattactcattaatcatatacttaatcactttgtctcataggttataatggagattaattagatgaataaacagaaacgaggaagcgaaggaccaaaccatcaaactgtaagctacagaagaccaaactatacaatcacaagattcaaagaaaaaacagagaaacatgcaattaccggtacaaatcaatgaaaacctaactttatcaacaacaatcaattgaaccaaaaaacgaaaatatgtaatcactagggagttatttaccataagaaggagggaacgccccaagaattcgccgagtcggcgccat

SEQ ID NO: 4 - DNA, 2223 bp, Oryza sativa
aagcaaaccaatcatcacctcttcttcttggggtgttcttgactggaaggtttctttggtgatggcgaaggcgagcgtggtggtgcctgagcaggtgggcgcggcggcggcggcgcaggtggggtgcccctgtccgggcacgacgctgttcccgtacccgccgccgcgcgccgggatcgccgtgcggcgcaagtgcctgcaggcggcgcagcagctggagctcggcgccgggctgcgcggcggctgggtggagtccatgcgggcgtcgtcgcccacccacgccaaggccgccgccgccctcgccgccggcgtcgacgaggagcacgccgcctggatggtccgtttccgttcaccgattgatcgatgttcgtcgcgttcttggcgcgcgcgcgctgacactgacatgaaccgtgcatttccgttcgtctttgtgcaggcgaggcacccgtcggcgctgggcgagttcgagaaggtggtggcggcgtcgaaggggaagcagatcgtcatgttcctcgactacgacggcaccctctcccccatcgtcgacgaccccgacgccgccttcatgagcgagacggtgagcttgagctcccctcccctgtcacctactctgctcctccactcatcatcatctcacacctctctccttcctcatcagatgcggatggccgtgcgcagcgtggcgaagcacttcccgacggcgatcgtgagcgggcggtgccgcgacaaggtgttcgagttcgtgaagctcgccgagctgtactacgcggggagccacggcatggacatcaagggccccgcctcccgccacgccgccgccaagtctcctccccacaacaagggagtcctcttccagccggccagcgagttcctccccatgatcgagcaggtgcaccagcgactcgagcaggccaccagctccatcccgggcgccaaggtcgagaacaacaagttctgcgtctccgtccacttccggtgcgtcgacgagaaggtaactgatcgatctgcaagctcgattggttgattggcttttttcatttggtgatcaattgatgatggaattgggggtgcagagttggggggcgttggcggagacggtgaggagggtggtgagggagttcccgcggctgcggctgagccaggggaggatggtgttcgaggtgcggccgaccatcaagtgggacaagggcaaggccctcgagttcctcctcgactcgctcggtacgtgcagtgtgttacaactttgccattcctcgtcggcaaaaaaacccattggctctgctccgccgcaggtttcgccgactgcagagacgtgctgccggtctacatcggcgacgaccgcacggacgaggacgcgttcaaggtaaataaatactaaccgacaaaaattactgcatgctgccacgctacgactacgtgtagcagcagcagtaacacgagacgctaccactacttcgtttgcaagtggttcgacgaacgtacggccggttcgtcgcgtgcagctagcgacaacgtacgtcttcttctacgttggactaaccggcgagccgtgtgcatgatccgggcaggttttgcggcggcgtgggcagggcgtggggatcctggtgtccaagcaccccaaggagacgagcgcctccttctccctccaggagcccgccgaggtaattaagcaaaaccactcgtacgcacgcatgaaaacgttcgtatcactctgctcatgtttgtgttgcaattgcgacaacaggtgatggagttcttgctgcggctcgtggagtggaatcgcctgtccaggacacggttgaggctgtaacaattgatgatcatctggcatcagctaatttaaccggcgaggctagctagagagaagcgcgtgatctgggccgtccgagcgattacatcggcagggtaacccgtgacgctgatcgatcgtggattctacaccaacacaggtgctcgaaaatggtgtccacattgcagaagcgcagagagctaattaatcaacgacggacgagagagactgatggctgtctggccattgttgtgccataatcctgtttagttcttcacctttcacctttctcccttcttcttttttcccatttggggccccccttttggtaccaaccatgtaaattccgtactactagtaccttgtcatgcacaagaggaagatcaatgcaaataatgaagagcaactaatgcaagtatatactcatcagcaca

SEQ ID NO: 5 - DNA, 2875 bp, Oryza sativa
attgcagcattaagtgttgatcgatcggagatggcgactcgggagggaggaggaggaggaggaggagggcggccgccgctgcgagtcgggcggacgcaggagtaccggatggggagggagacgcagctgctcgccgctgagggctcgccgccggtcagcctcttcgtgctgtgcggcgaccgcttcgaggcggcgcggctgttccgctccggcggcctgtcggtgcgcatggcccgcgtcgagggccacccggtctccatggcctcctgcgccgtcggcgaccaccactggatgctgtcccgggacgccctcgtcgcgcgcctcgacgcccgcgtcttcgtcttcgagatgcccggattcttctacgccgtcgtcgtcccttccgacgccggcgtcggcggcggtggcgccgagaggaagtgcgccaccctcgccgagatcttctcccggttctgctcctaccacgatctctccaccacgcagcaaggtattccaattaacatgcaaattagaacgcgtgtgattaccaattaatcctactagttaattacatattgtgcgatgcgtatcaaattatcttcatgcatgcgtgcatatatactaggttgattactgtacacaacaacatgtactagatcaaaaacattagttagagcaagtacatagcaggctataagccgaatatgatatgcgatgaaaagagaagagagaagaaagcaagctatttgtatctagctagctgtaacatggattctaagacgttatgtccaacatattacttcctccgtattttaatgtataacgccgttgactttttaacaaacgtttgatcttttgtcttactaaaaaaatttatgtaaagattattttataatattgaaatctgatgcgtttagtcatgacttaagtattttttgtttgcataaataatttactaaaggagatggataaataggggttaaaaagttaacagcgtcatacatgaaagatactgtaggtatatgcagtattattgtatgaatggactgacagaatatatatggatgatttagatctaacatttggcttatgctatttagggacagttgcatccatgcccctacatttaacctcaactgctgttttaccctcactttttagggtgttgccattttacccccattttatttaaaactccctcatttctttttaacaccgattgaggttgcgtcccacaccttttttagaaaaacaaataccaaataaaaaatgaatcttatgaaatatcgatttaccgttctatcctcgaggcaaatcgataaacatccaaccattatctccatctcccaccggattgtgcggaggcggaggcggaggcggccgcgggcgcgcgccccccaatgccgcgcttccacccacacgcacgaggtgccgccagaccgttatcagcgccctgccggacaaggtgctccgcctcgtgctgttgctgctcccggcgcacgaggcggtggcgacgtgcgtgctcgcccgtcgttggctccaccactggaaggaagcaccgggcctctccatggattggtggggttatcaggagttggacaggttcatcagcttcgtggatcgcgttttcacgctccgcagctacaacgcgcctctgaatcactgccatttctacatgtatttcctgaggttagtgcctggaagggagcggctcttcgtccgctggatccggcaagccttgcgctgccaagctcgggagctacgaatctgtttgatacttactccaagatgcctaacatgactctcatctctcagcacctcacaaggtctagagcattgaccatgaaggctagtatttgggatttttgtttaaattttgtaattgcctatatttgtgattcttttttgttaaagtaaaaaacatcacaagagatgcttatatttggtcaaaatgctcatcacacaagctgaaatcacaaatcacaaattaggagcaaaatcacgcaaaactgaaaaaaatagggcaaaaatatattaaccacccaggacataaatgtccttttttttaacaactagcaccgttaaaatcggccgttagaaatggaggtacatggttgatttagatttaaaatgtggcggtaaaactgcaagccctaaaaagtaggggtaaaacaataattggcttcgaaagtgagtgcaccgatgcaattgcctcatactattaaaacttgctctaaaagtagtgattagttttaacactgttgcagtgagacgtgtcgagtcctgattaatttcttcgttttgttctcgatgtttcgttcaggtgaagacgaagccggcggcgatatgaaccaacactccaacccgtgggtgcgcgcccacgccaggatacagcgtctgaagaagcccacctcgccgcccgccggcgccggccaggccaccgccgacgcggcggccgaacgcgccggcgccggcgccgttgtcggactcgcgagccagctggagcgcgccgtacgcacgtcggcggtcgtcaagctgctcagccgctccctcctcgccggcgcgctccagcccgcgcgccacctgatgatcaccctcgccgccgccgccggcggcgctgccgccaataatgccgccgggaccagcgccggcgcctgtggctcggcggcggcgctgccaagcaagtcggtcgtgtccgaccttctggaggccatcgagacgagccgtacgtcgccgcggcgggaggcggcgcggagggcgggcggcggcgccggcgggcccggggtggtggagcctcaacgtggagggcgtaatgctgctgctcagggtcgtccaggcggtcaggggggcggaagctgccggcgccggagaagaggacgcgcgacgaggcgagcga

SEQ ID NO: 6 - DNA, 2071 bp, Oryza sativa
ccttagatggggctggaaaggggataattttcttattggtataataatctaattgtgaaactattggacacttatttatgaaatgtgctcaggcttgatttgtggacaacgtaattaatatcactttcaatttgggtgcaatttagaatatcccacacatatttgagggatgaaacaattttggaaaagaaatgaggaagctaatagaggtaggtgtataaccgttgttccgtgagcaatttggacaaaaagagaaatggtgcttttttaaaaaaaaatttactgctaatagaggtaggcttgattactcgcccgttggagcgaatcagacatgtctatttttgagtttcatcccaaactattttaaataaatcacaggaagagacataagaaatataattttatgagtgagggatgtgggtaggtcagacgctaaataggatggcaagtgacacatataatacgaggtatggccaacgaccttggagaagaagactaagatgaattctctaagtcttggcatgaaggttttatttaagaagaaatgagtgaaagaagtccagggttttggaaaaatgctacttatgtcatctaattttggaccattttctatttttcggttgttaaatacaatacttatgttaagtgttttttctctctgtagagcaacttgaaagacaatgggatggcattattaagtcaagtgtcgttgataatgggattatgtactatttacccatttatgtaaatagaaatgatagaggccttgacctctctttttggaagaagataacacgacttgagaagcatactaacatagactatcttcatgatagtgtgagtgtgatgtgacgtctcaatgccatttgaaatctgtaggaagccaacgacatagtctatttgtaccaccataaacattatctgcatacatattcatactagtaccaagtctaaaatgtcaaatgaaaaagttggagagaaagataaacatatattggggttgagagagggacaatacaataatgggtggagaaagtggataagtaaaggaacaccaatatttgtgtggacaatgaggctatcatgttacgtatttttatctcgacagagggaaactattttaatcccttgaggggatgtctcctcattgtttgcatgtcacttaaatggttatgaaaaaattgaaaaaatttgagaagatgtattaacatgtgatatatcacttcacaaacatgcaagttcaaattcaacttctacatctcacaatgaaaaataaataaatttgactgtgaatatacgttaactagttgtagtttaatttgttttttcgttgcgagatgtagaagttgaatttgaatttgcatgtttgtggagtgatatatcacatgttaatacatcttctcaatttttttttaaattttttcataaccatttgagtgccatgcaaataacgaggggacattctttcgagggatcaaaatccactcccctcgacagataaataaccaattttttttgcgaagaacaataacttttatgacattattaatacagatgagttttatatttataccaaactccaaataaaaatatttagcagtatttgctactcgtgttaatctctacagtacaagttctcaagaaaattacactttgccccgtggggagttctaatcttccagtatttctcgtcgctcccgatgcaattgaaaaaatacacacgagttgtaacgaacaaaaaggaaaaaccgcacgggtttatcatcaaagctggatctgcacaacaaacagcctcgcaacagaaagagaatcaccacgggacaaaggcggtgacgtgtcacgcggcgcggccccacacgtccgcgtgggcccggcacgtgccccgcggcgcgggggccacctggcgccgagctgggctcgcgcgggtgtggcgacgtcggacgcgtccaagacatcggcgtggcccgtggccaccaccaccaccggcgggccccgccgtcgccgccgccgccgtctccctcctccctctccaactataaatacctcactcctcctcttcccgcctccacac

SEQ ID NO: 7 - DNA, 1128 bp, Oryza sativa
atggcgaaggcgagcgtggtggtgcctgagcaggtgggcgcggcggcggcggcgcaggtggggtgcccctgtccgggcacgacgctgttcccgtacccgccgccgcgcgccgggatcgccgtgcggcgcaagtgcctgcaggcggcgcagcagctggagctcggcgccgggctgcgcggcggctgggtggagtccatgcgggcgtcgtcgcccacccacgccaaggccgccgccgccctcgccgccggcgtcgacgaggagcacgccgcctggatggcgaggcacccgtcggcgctgggcgagttcgagaaggtggtggcggcgtcgaaggggaagcagatcgtcatgttcctcgactacgacggcaccctctcccccatcgtcgacgaccccgacgccgccttcatgagcgagacgatgcggatggccgtgcgcagcgtggcgaagcacttcccgacggcgatcgtgagcgggcggtgccgcgacaaggtgttcgagttcgtgaagctcgccgagctgtactacgcggggagccacggcatggacatcaagggccccgcctcccgccacgccgccgccaagtctcctccccacaacaagggagtcctcttccagccggccagcgagttcctccccatgatcgagcaggtgcaccagcgactcgagcaggccaccagctccatcccgggcgccaaggtcgagaacaacaagttctgcgtctccgtccacttccggtgcgtcgacgagaagagttggggggcgttggcggagacggtgaggagggtggtgagggagttcccgcggctgcggctgagccaggggaggatggtgttcgaggtgcggccgaccatcaagtgggacaagggcaaggccctcgagttcctcctcgactcgctcggtttcgccgactgcagagacgtgctgccggtctacatcggcgacgaccgcacggacgaggacgcgttcaaggttttgcggcggcgtgggcagggcgtggggatcctggtgtccaagcaccccaaggagacgagcgcctccttctccctccaggagcccgccgaggtgatggagttcttgctgcggctcgtggagtggaatcgcctgtccaggacacggttgaggctgtaa

SEQ ID NO: 8 - DNA, 1212 bp, Oryza sativa
atggcgaaggcgagcgtggtggtgcctgagcaggtgggcgcggcggcggcggcgcaggtggggtgcccctgtccgggcacgacgctgttcccgtacccgccgccgcgcgccgggatcgccgtgcggcgcaagtgcctgcaggcggcgcagcagctggagctcggcgccgggctgcgcggcggctgggtggagtccatgcgggcgtcgtcgcccacccacgccaaggccgccgccgccctcgccgccggcgtcgacgaggagcacgccgcctggatggcgaggcacccgtcggcgctgggcgagttcgagaaggtggtggcggcgtcgaaggggaagcagatcgtcatgttcctcgactacgacggcaccctctcccccatcgtcgacgaccccgacgccgccttcatgagcgagacgatgcggatggccgtgcgcagcgtggcgaagcacttcccgacggcgatcgtgagcgggcggtgccgcgacaaggtgttcgagttcgtgaagctcgccgagctgtactacgcggggagccacggcatggacatcaagggccccgcctcccgccacgccgccgccaagtctcctccccacaacaagggagtcctcttccagccggccagcgagttcctccccatgatcgagcaggtgcaccagcgactcgagcaggccaccagctccatcccgggcgccaaggtcgagaacaacaagttctgcgtctccgtccacttccggtgcgtcgacgagaagagttggggggcgttggcggagacggtgaggagggtggtgagggagttcccgcggctgcggctgagccaggggaggatggtgttcgaggtgcggccgaccatcaagtgggacaagggcaaggccctcgagttcctcctcgactcgctcggtttcgccgactgcagagacgtgctgccggtctacatcggcgacgaccgcacggacgaggacgcgttcaaggttttgcggcggcgtgggcagggcgtggggatcctggtgtccaagcaccccaaggagacgagcgcctccttctccctccaggagcccgccgaggtaattaagcaaaaccactcgtacgcacgcatgaaaacgttcgtatcactctgctcatgtttgtgttgcaattgcgacaacaggtgatggagttcttgctgcggctcgtggagtggaatcgcctgtccaggacacggttgaggctgtaac

SEQ ID NO: 9 - DNA, 1405 bp, Oryza sativa
atggcgaaggcgagcgtggtggtgcctgagcaggtgggcgcggcggcggcggcgcaggtggggtgcccctgtccgggcacgacgctgttcccgtacccgccgccgcgcgccgggatcgccgtgcggcgcaagtgcctgcaggcggcgcagcagctggagctcggcgccgggctgcgcggcggctgggtggagtccatgcgggcgtcgtcgcccacccacgccaaggccgccgccgccctcgccgccggcgtcgacgaggagcacgccgcctggatggcgaggcacccgtcggcgctgggcgagttcgagaaggtggtggcggcgtcgaaggggaagcagatcgtcatgttcctcgactacgacggcaccctctcccccatcgtcgacgaccccgacgccgccttcatgagcgagacgatgcggatggccgtgcgcagcgtggcgaagcacttcccgacggcgatcgtgagcgggcggtgccgcgacaaggtgttcgagttcgtgaagctcgccgagctgtactacgcggggagccacggcatggacatcaagggccccgcctcccgccacgccgccgccaagtctcctccccacaacaagggagtcctcttccagccggccagcgagttcctccccatgatcgagcaggtgcaccagcgactcgagcaggccaccagctccatcccgggcgccaaggtcgagaacaacaagttctgcgtctccgtccacttccggtgcgtcgacgagaagagttggggggcgttggcggagacggtgaggagggtggtgagggagttcccgcggctgcggctgagccaggggaggatggtgttcgaggtgcggccgaccatcaagtgggacaagggcaaggccctcgagttcctcctcgactcgctcggtacgtgcagtgtgttacaactttgccattcctcgtcggcaaaaaaacccattggctctgctccgccgcaggtttcgccgactgcagagacgtgctgccggtctacatcggcgacgaccgcacggacgaggacgcgttcaaggtaaataaatactaaccgacaaaaattactgcatgctgccacgctacgactacgtgtagcagcagcagtaacacgagacgctaccactacttcgtttgcaagtggttcgacgaacgtacggccggttcgtcgcgtgcagctagcgacaacgtacgtcttcttctacgttggactaaccggcgagccgtgtgcatgatccgggcaggttttgcggcggcgtgggcagggcgtggggatcctggtgtccaagcaccccaaggagacgagcgcctccttctccctccaggagcccgccgaggtgatggagttcttgctgcggctcgtggagtggaatcgcctgtccaggacacggttgaggctgtaac

SEQ ID NO: 10 - DNA, 424 bp, Oryza sativa
aagtacaggtagtagtgacaacggcattgtgtaatactcatgaaaagcaagcaacggcagcaggagaagtctctttcagctgtcaatagttttgccgtggatcacgagtagagagtcatcaaatcagatgtttattagacggagcgtatcagcgtatcgtgtactcctgctagtgttgctagtgtgtttgtgctgcctgcaagcgcaatgcagcagcaagcaactgcagtgagatgatcgaatcgaatcctgcaaggggaaaaaggaatcatcagtgaatgaaatccaatccctttcggttttggttttggttttcctgcctgcctggttgcttgctttgcagtttgccttgtggtcgtggaccgagctgctatgctattgctagctttcctcctttcttttcgcgtatctcgatgactcgaca

SEQ ID NO: 11 - DNA, 636 bp, Oryza sativa
tttttgtgtgggagccacaacaactcattccttttataaattaaattatattttttgtcagtttatttctcttactgtcctttgatagccatggaatgcccctgtatctttccgtttccttttcgatgctgtatgggctatggcgaaccttccggtggtaattttttttttataattacacagtaaaattcgcgtcctttcatgagttaatcctgcaagtaaagttttatccgctaattatcttcttcagatcagattccttctttccctgacgaaaaagtacagcatcagtaatccaggggtccatttccatttggtgattatacatgcatcaatggattgttttcagcaaattactagccgacgataccgtagcattctcgtaattaatgacagagattttgtagtgtcgccgatggattctacagtggacactatcatgatcatattgtacttatcatggtaattaatattcctgataatttcccctgcgaaaacatcagatcgccttctctcctgtcgtcgttggattgctgtaaattgcaaacggtcgttcagaaaattgtctggtttcctagtattttgccgtaagaggatactagaattgactaaactggccaagtcaaacttcaaact

SEQ ID NO: 12 - DNA, 904 bp, Oryza sativa
tctgatctattatacacaaaaagattatacagagtacatattcatgcttagagcaaaaattatttctaatcacaaactattcgtgaaggattacttatatgacacctagccttataaaacatcgagaacaaaacgaagaaattaatcaggactcgacacgtctcactgcaacagtgttaaaactaaccactacttttagagcaagttttaatagtacggggcaattgcatcggtgcactcactttcgaagtcaattgttgttttacccctactttttagggcttgcagttttaccctcacattttaaatctaaatcagccatgtacccccatttctaacggccgattttaacggtgctggatgttaaaaaaaaaggacatttatgtcctgggtggttattgtatttttgccctatttttttctgttttgcgtgattttgctcctaatttgtgatttgtgatttcagcttgtgtgatgagcattttgaccaaatatgagcatctcttgtgatgttttttactttaacaaaaaagaatcacaaatataggcaattacaaaatttaaacaaaaatcccaaatactagccttcatggtcaatgctctagaccttgtgaggtgctgagagatgagagtcatgttaggcatcttggagtaagtatcaaacagattcgtagctcccgagcttggcagcgcaaggcttgctggatccagcggacgaagagccgctcccttccaggcactaacctcaggaaatacatgtagaaatggcagtgattcagaggcgcgttgtagctgcggagcgtgaaaacgcgatccacgaagctgatgaacctgtccaactcctgataaccccaccaatccatggagaggcccggtgcttccttccagtggtggagccaacgac

SEQ ID NO: 13 - DNA, 2310 bp, Oryza sativa
atggcgtgggcatcggcggcctggggagaaaggaagatggcggcagcggtggaagaggaggcggtgaaggaagaggcggcatcatttataggtcctaacctcgttctcgttcccgttccgtaccgtcaacctcgtcgtcatcctccgctccaagccaccttggccagtccgcaacagcagcttgatgatggcctcctcgagtcctccgcctcccatgcttaccactagatccgtgccgtcccattctgtgccaccccgctccatgccgctgagcggccgtcccgctcgtagtctctgtctctccgagctccacgccggcgcgccggccaaggtaggatggacgccggtcgtaggtcaggggaggagtcctcggtcgccggtagggggaggagtcaggacatcagccggtgaaggaggggagggagccaaagtcgctggctcgttgtcgggagtgagtggaggagaggtcgagaggagataaggggggagagagacgggggagggaggaatttataattttgttttggagtctagggtctttggtataaattttgaacctctacccaaaatgaaaaaagtatgaaaactaaatcaaataagtggtagggactgaaatataaatttaaagccacacctaaaaccacaaattttggacctgtgaccctagcctatgtggcagtctgaggctcctcatttgactgggacacaatgtgaatggctttagtagtttacatcttttgattcttttagttaaaaacttattttatatatagagctctaagaaaacttacaccctctgtgctatgagaaactaacttatgagaattaatctggacaaatgtttatctaaatttatcttaaaaatagttttttttaactattctaggaatcaacgttttttctcggtgcaaaggtaatgataccgagatgtcttggtgtcaatatcgccaataacgttagcgttgacaccctagccccatgtgatagctaaatcattgacgtttagatgttggacttcgataacaggatcactagcgtcgggaaaaaaggtctaattttaaatatgtgcttttaagaatctatttacaaaataatttttaaaaggctcgaaacataatagaatccgacagtagtagggtaagtctggttgcttggctgggcttggcgttccatgacttctatccttgctgctacacgggaaaaatatacagtgcaaaccacatgacacatatgtagtggaaacctgaaagctatagttacataaaaaaatagacgtattcgtccacgtcattatgagtaaaattttgacccctaatgatggtagttctcgatgcaaaggtattcatacgtctattttttaatttgatggtagtttttgggtgtctactatatacgtaatttccggtgtatatttatactactctatgctgaatgctgaagtaaatgtgaaatggcaggcacaccagccactggacaaggaggtagggtcggcctcatctgcaactgatcaccggtcaccaccaccgttgggtaagtagcagcagcgtgtagtgggcaagtctcaaatacagtgatccttggtggccggtacaacattcttgatggcaaagtggttgctcacatacagtgatccataactagtgctttatttttgttgggaagtccaaatgatactcttttttgttgaattgaaaatagggccaattggtttggagccggtttttttgtcataccaaaatttagtgccaaaatgttggtatgttttagtaccactagattgatagtatagaatttgtatagtatgtgtaaaaattgatagcaaacaaaacatccatataacattattgaagttgccaaatttttttggtaggacaaagcactataaaaaaattattttcccatacaatcaaaaccaattttcgcatgtggttgggtcgccgcctgctagggcagcgcacctgtccgcgaaaatccgccttctgcgggcggtaaattatgtcgcccaccctcggaaagaaaaatcgctaaaaccctatctagctcccatccacaggtgacattaacactatagtctgctatcgctcgtcaccgtcgttgccacagaggccccgcagctcatactcatcgtcgtcgccctgcccccatgccttcaccacctccaccgttgccccttctcaccccggtagcagcggcagcgtggatccgcctgtcacggggatgctcgacagcggaacccattgcggcgccacccgacggcggatcccgcctagaggggagttagctgccgaatcctcgtgggagaggagttga

SEQ ID NO: 14 - DNA, 1248 bp, Oryza sativa
atggcggggaagaggggaaacgcggtggggcggctgcggaggccggtgaccacggaggcaagggaggcgcggtcggcggcaaggccgaggcagctcggggtcgcgggtgagcgccgtcggcgggtgaggcagctcgaggtcacgggtgagcagcaaagagaagaggagacgagtggttcaccgtcgccattgaggtcactgttcggcgctgaggcggaagcggtggcgctgctaattctcccttccctggagacggcggtgaggcctccccccctctctctctctctccctcctccttctccactcaggcaggcggtgagcgacaaatcctcctcggggaacgcatggcgcgcgtggtgggcagggcgaggcgggcggcaccgaggacattggaggcggcaggctatcgtgacgagtttgccgcccatccccgtcgtccccgtgcccgattgttcctccgccgcctccgctccaatctcctctccttcccgatgccctcgccggcggctctaccccactggtcccgtcacgacgtgccctccgcaatctcgccgtgcaccagagggaagaaagagagaagagagattttagcagtactccgactgccgccgcatcctctgcgtgctcgctacgccgcccttgtctccgcctccatgccgctgccggcacgtgccgccctcgcttccacctccttgccgctgccggtagtgccgctctactgtgtcgctccgtctccccctgccgccgtgcgctttagaaggagggagaagagcgccgcgtcgttccctctccccagccaccacccgctttagaaggggatggagaatagagaggtggtggggattctaacgagtgggatctagcgattttttttaaaaaaactgatgattggactgtcacgtgtgctatttgaatcctaaaccgcttgcaacagtgagcgatatctggttttcgagtttagggggtaattcggtcgatcgcgatagattaagggggtaattcgtactttcctttatattatggattgagaacaagaggatagcttattcttcaacgcatccttgtgacaatctcctgcactcccagataaacagatcatgcatgagcatatatatcgatcttgttagaagatatagctagctttgattgtcagtcaaagatatggctccctttttatcaccatcacagtcaggggcaacatggctagaggtcacttcactatcaaaggtcatggatcgagtatactga

SEQ ID NO: 15 - DNA, 3419 bp, Oryza sativa
atggcgccgactcggcgaattcttggggcgtcccctccttcttatggtaaataactccctagtgattacatattttcgtttttggttcaattgattgttgttgataaagttagggtttcattgatttgtaccggtaattgcatgtttctctgttttttcttgaatcttgtgattgtatagtttggtctctgtagctacagtttgatggtttggtccttgcttcctcgtttctatttattcatctaattaatctccattataacctatgagacaaagtgattaagtatatgattaatgagtaatatgttgtatatgcattggggcagtgttgttgtatgcatggagcctgggcaattgcctatggtggcccggcgctgcctccgccactgctttggtgttttataaggctaggtgtcatataagtaatccttcacgaatagtttgtgattagaaataatttttgctctaagcatgaatatgtactctgtataatctttttgtgtataatagatcagataattatttacgatttgacacatcagaatggtttgtcttgtaatataggctgcgaggatgaacagttcacaactgaactacatcatggtggttttttctctggagttggtagtagcaaaatttatttgaatgaaaaggtggattggtttgattattgtgacagtgacacatggtcttcattatggcttgatgattttattgagcaactaggatatgatatgtctgagaaggccaaggtttattggttattgcctgggaagacattcagtgatgggctaaggagaattaactctgatgcaagcactaacagcatggttgtattagcaccaaggtttaggacattgcttttgtatgtggatcaccaagacattcttgaatccatcaattgggatgatgttgctctttctacccctgcttctatacctatcgtggctagccgtgagaggtctacaatgaatcaaaaagattcaggtgttatacctatagaaggtcatgacattgacgatgacgatgacatcgatgtagatcctgactttgttgacagtgattatgaggctaacaagggggatgatgatgtttttgacaactgtgttgagatagaagaagtggctagcagcaagatggttgttaagcttcctgaacatgactctgatgatgaaggcttgttgcttccagagtcatctgatgaagataatgaaggcattaagttgaaatttaagaactttgtggctgctgtggacatggagtcaccaatctttaaggttgggatgttgttctctgatgctattgaagttaggcaagcaataaaacaatacagtgtgaagaatagagtcgcaattaaatttacaagaaataccaagaaaaaaattgaggctaaatgtaaagaagagtgctcatggatgttggatgtagctgaggacagcaggacgaattgtcttatggtaaaaagatatatagatggccacaaatgtcagaaggagtgggagttgaattatgtaactgctaggtatcttgcaaatagatatgttgaaagctttagggataatgataggatgacactcaagagtttctctaaggtagttcaaaaggaactaaatgtaacaccatcaagacataaattaggtcgagcaaggaaactagctatgaaagcgatatatggtgatgaaatagcacaatacgatatgttgtgggattatggacaagagcttaggacttcaaatccaggtagtaaatttttcttgaacctacaaaatatgtgcttccatacatgctatgtgtcatttgatgcttgcaaaagaggattcctaagtagttgtagacctatcatattccttgatggatgccacatcaaaaccaagtttgggggacatttgttgactgtagtaggcattgatcctaatgactgcatctatcctatagccatggctgttgtggaggtggagagtagagctagttggggttggtttttgaatactttgaaagaagatctccttatcgacaacactggaccttatactatcatgacagacaggcagaaggtgtgactttattccatcatatttgattttgttcaacttgtcatttttaaaatatttgaattatttactgttcaatggattggtactcacccttcctttttaaattttgtagggattggtaccagctgtgagggataaattctcagattcagaacaaagattttgcgttaggcatttgtaccaaaattttcatgttctatacaaaggggagactctaaagaatcagttgtgggcaattgcaaggtcaagtactataccagagtgggaatttaacatggaaaagatgaagtctcttagcaaagatgcttatgcctacctggaggaaataccacccaaccagtggtgcagagcattctttagtgatttccccaagtgtgatattctactcaacaacaattccgaggtattcaacaagtacattcttgaagccagagagcttccaattttatccatgcttgaaaagattaggagccaaatcatgaataggatacataccaaacaagaggagtgtgtaaagaagtggtcgggaaacatatgcccaaaaatatagaaaaaggttgataaaaatgcagaattgtcaaatacatgctatatattacctgctggtaaaggggtgttccaagcaactgataaagaacaccaatatattgttgacctcatggcaaaacactgtgaatgtaggagatggcaattgatagggataccatgcaaccatgcaatttcttgcttgaggagtgagaggacaaaacctgaagatgtggtatccttctattattcaactgaaaaatacatggaggcctatggatttaacataatgcctgttagggacaaggcatcttgggtgaagatgaatggaccaaaggtgaacccaccgatttatgagaagaaagttggaaggcctaggagatgtagaagaaagcagccacatgaacttgaaggaggaacaaaaatatccaagcatggtgtgcaaatgcattgtagctactgtcaaggagttaatcacaaaaagaagggctgcaagaagaggaaggaagatataaaagcagaaaagcagcaagccagtgctgctccttcacaacaacatggtaatgaagattttatgcctctgcaggtcatgcctgaagttctgaacagtcagactagcattttaaccagccaacctgtcacgccctgaagttcccctcccttgctctaaattcataaaataaatcgtccgaagtaattgtttaatttaacctagggatgaatccctaattaataaatgcaaataataatcggaatcgatatgtggaatttttcttagattctacatgtcaaaatatgctaacacgatttttagtggaattttcagagccttggaaataa

SEQ ID NO: 16 - DNA, 2213 bp, Oryza sativa
aagcaaaccaatcatcacctcttcttcttggggtgttcttgactggaaggtttctttggtgatggcgaaggcgagcgtggtggtgcctgagcaggtgggcgcggcggcggcggcgcaggtggggtgcccctgtccgggcacgacgctgttcccgtacccgccgccgcgcgccgggatcgccgtgcggcgcaagtgcctgcaggcggcgcagcagctggagctcggcgccgggctgcgcggcggctgggtggagtccatgcgggcgtcgtcgcccacccacgccaaggccgccgccgccctcgccgccggcgtcgacgaggagcacgccgcctggatggtccgtttccgttcaccgattgatcgatgttcgtcgcgttcttggcgcgcgcgcgctgacactgacatgaaccgtgcatttccgttcgtctttgtgcaggcgaggcacccgtcggcgctgggcgagttcgagaaggtggtggcggcgtcgaaggggaagcagatcgtcatgttcctcgactacgacggcaccctctcccccatcgtcgacgaccccgacgccgccttcatgagcgagacggtgagcttgagctcccctcccctgtcacctactctgctcctccactcatcatcatctcacacctctctccttcctcatcagatgcggatggccgtgcgcagcgtggcgaagcacttcccgacggcgatcgtgagcgggcggtgccgcgacaaggtgttcgagttcgtgaagctcgccgagctgtactacgcggggagccacggcatggacatcaagggccccgcctcccgccacgccgccgccaagtctcctccccacaacaagggagtcctcttccagccggccagcgagttcctccccatgatcgagcaggtgcaccagcgactcgagcaggccaccagctccatcccgggcgccaaggtcgagaacaacaagttctgcgtctccgtccacttccggtgcgtcgacgagaaggtaactgatcgatctgcaagctcgattggttgattggcttttttcatttggtgatcaattgatgatggaattgggggtgcagagttggggggcgttggcggagacggtgaggagggtggtgagggagttcccgcggctgcggctgagccaggggaggatggtgttcgaggtgcggccgaccatcaagtgggacaagggcaaggccctcgagttcctcctcgactcgctcggtacgtgcagtgtgttactactctacaactttgccattcctcgtcggcaaaaaaacccattggctctgctccgccgcaggtttcgccgactgcagcgacgtgctgccggtctacatcggcgacgaccgcacggacgaggacgcgttcaaggtaaataaatacttgtactactaaccgacaaaaattactgcatgctgccacgctacgactacgtgtagcagcagcagtaacacgagacgctaccactacttcgtttgcaagtggttcgacgaacgtacggccggttcgtcgcgtgcagctagcgacaacgtacgtcttcttctacgttggactaatcggcgagccgtgtgcatgatccgggcaggttttgcggcggcgtgggcagggcgtggggatcctggtgtccaagcaccccaaggagacgagcgcctccttctccctccaggagcccgccgaggtaattaagcaaaaccactcgtacgcacgcatgaaaacgttcgtatcactctgctcatgtttgtgttgcaattgcgacaacaggtgatggagttcttgctgcggctcgtggagtggaatcgcctgtccaggacacggttgaggctgtaacaattgaatttaaccggcgaggctagctagagagaagcgcgtgatctgggccgtccaagcgattacatcggcagggtaacccgtgacgctgatcgatcgtggattctacaccaacacaggtgctcgaaaatggtgtccacattgcagaagcgcagagagctaattaatcaacgacggacgagagaaactgatggctgtctggccattgttgtgccataatcctgtttagttcttcacctttctcccttcttcttttttcccatttggggccccccttttggtaccaaccatgtaaattccgtactactagtaccttgtcatgcacaagaggaagatcaatgcaaataatgaagagcaactaatgcaagtatatactcatcagcaca

SEQ ID NO: 17 - DNA, 4116 bp, Oryza sativa
agctcgtacaacctcgcatatactccaacatgatccctctctagctaacgtaattcccttctcttttctctccgatccggctctcgttgtggtaagcactacgtaccatagtttaactggtagctagcatagagtgagtggtgcatacatacatggtcattaaccttaacacgtgccgttgtttaacctagcgaataatattagctgcatggaaaaatatccgggcaaaaaatatctatctagccacgagccaacgatctatcactgtctaataaaaaggaccagtatatttgccgtatggacatgctctttgcgtgcctgcagcttcttcttcttcctcttcttcttttgcagtacagagtgaaattgaatctagtagcttaatttgtgtgatcaagcagcattaagtgttgatcgatcggagatggcgactcgggagggaggaggacgaggaggagggcggccgccgctgcgagtcgggcggacgcaggagtaccggatggggagggagacgcagctgctcgccgctgagggctcgccgccggtcagcctcttcgtgctgtgcggcgaccgcttcgaggcggcgcggctgttccgctccggcggcctgtcggtgcgcatggcccgcgtcgagggccacccggtctccatggcctcctgcgccgtcggcaaccaccactggatgctgtcccgggacgccctcgtcgcgcgcctcgacgcccgcgtcttcgtcttcgagatgcccggattcttctacgccgtcgtcggcggcggtggcgccgagaggaagtgcgccaccctcgccgagatcttctcccggttctgctcctaccacgatctctccaccacgcagcaaggtattccaattaacatgcaaattagaacgcgtgtgattaccaattaatcctactagttaattacatattgtgcgatgcgtatcaaattatcttcatgcatgcgtgcatatatactaggttgattactgtacacaacaacatgtactagatcaaaaacattagttagagcaggtacatagcaagctataagccgaatatgatacgcgatgaaaagagaagagagaagaaagcaagctatttgtatctagctgtaacatagactatgatatgcgatgaaaagagaagagagaagaaagcaagctatttgttatgtccaacatattactccctccgtattttaatgtatgacgccgttgactttttaacaaacgtttgatcttttgtcttactaaaaatttttaatcttttgtcttactaaaaaaatttatgtaattatcatttattttattgtgatttgatttatcatcaaatgttctttaagcatgacataataagcatgacataagtattttttttatttgcataaaaaaattgaatcatgttcttttaagcatgacataagtattttttatttgcataaaaaaattaaataagacgaatggttaaatattggttaaaaagttaacggcgtcatacattaaaatacggagggagagggagtatatgggtataatacctgagaccaggtattaatggtgtagtatatttttatacttaattattgtattaattgactgttagaatagcgatagatgatttagatctaacatttggctatactattagggacaattgcatccatgcccctacttttaattccaactactgttttaccctcactttttagggtttgcaattttacccccatttttttaaaacgtcctcagttttaacaccgttaggttggtcccacatttttttagaaaaacaaatagaaataaaaaaaatgatttatgaaatatcaatttaccgttctatcctcgagggaatcgataaacatccaaccagctctcctctcccaccggattggtccttgcgcggcggcggcggcggccgcgtgtgtgcgcggaggcggaggcggaggcggcggcggcgccgcgcttccacccacacgcacgaggtgccgccagaccgcatcagcgccctgccggacgaggtgctccgcctcgtgctgttgctgctcccggcgcacgaggcggtggcgacgtgcgtgctcgcccgtcgttggctccaccactgggaggaagcaccgggcctctccatcgattggtggggttatcaggagttggacaggttcatcagcttcgtggatcgcgttttcacgctccgcagctacaacgcgcctctgaatcactgccatttctacatgtatttcctgaggttagtgcctggaagggagcggctcttcgtccgctggatccggcaagccttgcgctgccaagctcgggtgctacgaatctgtttgatacttaggggctgtttggttcccagccacactttaccattacttgccaacaaaagttgtcacaccttacctaaggtgaggtgatcaaattgttagccacaacttactaagtctaagggaatcttgccacacttttttgagccattgacacgtgggacccaatttgttggaggggaatcttgccacaactgtggctacaaccaaacacctgtcaaatctgtctaaccttaggcgttgcaaactgtggcaaagtgtagcttgcaaccaaacacacccttactccaagatgcctaacatgactctcatctctcagcacctcacaaggtctagagcattgaccatgaaggctatcatctctcagcacctcacaaggtctagagcattgaccatgaaggctatcatctctcagcacctcacaaggtctagagcattgaccatgaaggctagtatttgggatttttgtttaaattttgtaattgcctatatttgtgattcttttttgttaaagtaaaaaacatcacaagagatgcttatatttggtcaaaatgctcatcacacaagctgaaatcacaaattaggagcaaaatcacgcaaaactgaaaaaaatagggcaaaaatacaataaccatccaggatataaatgtccttttttttaacaactagcaccgttaaaatcggccgttagaaataggggtacatggctgatttagatttaaaatgtgggggtaaaactgcaagccctaaaaagtgggggtaaaacaacaattggcttcgaaagtgagtgcacagatgcaattgcccccatgctattaaaacttgctgattagttttaacactgttgcagtgagacgtgtcgagtcctgattaatttcttcgttttgttctcgatgtttcgttcaggtgaagacgaagccggcggcgatatgaaccaacactccaacccgtgggtgcgcgcccacggcaggatacagcgtctgaagaagcccacctcgccgcccgccggcgccggccaggccaccgccgacgcggcggccgaacgcgccggcgccggcgccgttgtcggactcgcgagccagctggagcgcgccgtacgcacgtcggcggtcgtcaagctgctcagccgctccctcctcgccggcgcgctccagcccgcgcgccacctgatgatcaccctcgccgccgccgccggcggcgctgccgccaataatgccgccgggaccagcgccggcgccggtggctcggcggcggcgctgccgagcaagtcggtcgtgtccgaccttctggaggccatcgagacgagccgtacgtcgccgcggcgggaggcggcgcggagggcgggcggcggcgccggcgggcccgggtggtggagcctcaacgtggagggcgtaatgctgctgctcagggtcgtccaggcggtcagggggcggaagctgccggcgccggagaagaggacgcgcgacggggcgagcgacgccgccggcctgagaggcggcggcatcatgggcggcggcggcggcggcgccgcgaggcggtggtgcggcggccggccgaagaagctgggcaacacggtgggcgcgtgtggaactcctgatctgaggctttgtgttaatgttcccaaaagtgaaccgagagagagagagagattattaataggtaattaagcggaggattaggttaatgatatagagtttaacaagttgatctgtactgttagatgcatgatcaggtactaatgcttggtcatagtgtttcttgttctgcttctaataatattcatgaatcggtgtttaatta

SEQ ID NO: 18 - DNA, 25945 bp, Oryza sativa
tctttttctgatttcaccttccaacttgcgaagatctgcctgttgagttgcttgtgtgtgggggcagagaaacacttcgctttccagaaaggagaagacgaacaaactcgtctgtctgactgtctctgcatcaacgtcactggatttcggctagcggatgcagatctcatctcaacggcgcgaagcttcgggcgaagatgttcttcaatattttccgcttaattttctaccatgttaatccgaaagcgacgctacccgtggaccggtcagagtgaggctctcagcgggggcagcggcaacgacggagcacgtcgccgacgagatgctacgccgtaatgttttttttttcttttttcatcgcctgatttggatttcgcatcgctcccgtcaggatgcactacacggaatcgcgttctttagcctcggtttcaatgacaggacaccgttgatctcacgcgaacgtgccgttgcgttttactgtgtgtactacaaggaatcgaatcttgcaaggtacagtatgatatccgtgacgggcaacgacgataccttgacaggcaggctcggtgcatcgatttctttcttcttttttttgatttgaatgttccgggcggagttggaaacagaggcgcgagagtggaggaattccccgccaaaaaatatctgagagattcgtagcagtaggtacagcattgcgaccgcaatagtgaagtaagatgctctttataaaatatatacatctcagcaatagactagattaatatgtacatcttagcaatagactagattaatagtaaaccacctcaatattatgtctacatggatatctatagctctatcatgtattgtctcatttttctctatagactatttttaagttagtagatagttttgctctctctttttatttaatatcttcgaagtagaaaaatatgctgacatggatctcttgtagagagcttatagataactattgtccctagtgcacatggctggcgggaaaccgcgtagcctcagcgtccgcgccagcgacgactctcgtactctctctctctctctcctctcgtctacaaaaaggatgggcctcattggccccacccacgtcgccctctggatgagggtggcattgaatgcgtgtgttggtgggccccaccaccacgcaggaggtgggcccacctgccagtgtcacaggggctcagtacgcagaaagagagagagagatcaggtaaaaatggaatggaaaggagagccgccaaggcggccacaggtgaaacgccgacgacgaccacgaccaggcgtttctctgactagtgagtgatacactgatactcctacaattaactgtggtgctttgtgctttggttggttgctttggcacgtggtaactggcaatgtggaacgcgcgctgtccgtcactagcgattcttgtagagtgtagtacaactatctcatcatattttcacctgatctaagggcccgatcgagagagtgagggtgggggaataatctctctggcacggaaaacggagcaggccattagcacatgattaattaagtattagctatttttttaaattggatcaatatgaattttttaagcaacttttatatataattttttttacaaaaaaaacacaccgtttaacagcttaggaagcgtgtgcgtgaaagacgaggtggtggggacgtttgcccgtgggaacgagcgcacccttaaagccgtgaggcgtcatcatcaatccggtggcaagttgggcttgtggcactgtccggctgcacactccagtagcagtagacggtactccttaggccattcgccgtgcgtcaccgtggcgttctacaccgactcgctaaacttgggcatgtgttcttcgtcagttttggcaggcatgtgtcaaagcgaggcgaggaactggcgaagaacggtgcgcacaacacgttgggagagggcgtgggcctatgcttcgtcgctcccaacgcaacggtggctggatctggtgacgtcgatggcgtgggcatcggcggcctggggagaaaggaagatggcggtagcggtggaagaggaggcggtgaaggaagaggcgacatcatttataggtcctaaccttgttctcgttcccgtttcgtaccgtcaacctcgtcgtcatcctccgctccaagccaccttggccagtccgcagcagcagcttgacgatggcctcctcgagtcctccgcctcccacgcttaccactagatccgtgccgtcccattctgtgccaccccgctccatgccgctgagcggccgtcccgctcgtagtctctgtctctccgagctccacgccggcgcgccagccagggtaggatggacgccggtcgtaggtcaggggaggagtcctcggtcgccggtagggggaggagtcaggacattagccggtgaaggaggggagggagccaaagtcgctggctcgttgtcgggagtgagtggaggagaggtcgagaggagataaggggggagagagacgggggagggaagaatttataattttgttttggagtctagggtctttggtataaattttgaacctctacacaaaatgaaaaaagtatgagaactaaatcaaataagtggtagggactgaaatataaatttaaagccacacctaaagccacttgcaatatggagaactcaaattttggacctgtgaccctagcctatatggcagtctgaggctcctcgtttgactgggacacaatgtgaatggctttagtagtttacatcttttgattcttttagttaaaaacttattttatatatagagctatgagaaaacttacaccttctgtgctatgagaaactaacttctgaggattaatctggacaaatgtttatctaaatttatcttaaaaatagttttttttaactattctaggaatcaacgttttttcacggtgcaaaggtaatgataccgagatgtcttggtgtcaatatcgccaataacgttagagttgacaccctagccccatgtgatagctaaatcattgacgtttagatgttggacttcgatatcagaatcactagcgtcgggaaaaaaggtctaattttaaatatgtgcttttaagaatctatttacaaaataatttttaaaaggctcgaaacataaatagaatccgacagtagtagggtaagtctggttgcttggctgggcttggcgttccatgacttctatccttgctgctacacaggaaaaatatacagtgcaaaccacatgacatatacgcagtagaaacctgaaaactgaaaactatagttacataaaaaaaatagacgtattcgtccacgtcattatgagtaaaattttgacccctaatgatggtagttcttgatgcaaaggtattcatacgtctattttttaatttgatggtagtttttgggtgtctactatatacgtaatttccggtgtatatttatactactctatgctgaatgctgaagtaaatgtgaaatggcaagcacaccagccactggacaaggaggtagggtcggcctcatctgcaactgttcaccggtcaccaccaccgttgggtaagtagcagcagcgtgtagtgggcaagtctcaaatacagtgatccttggtggccggtacaacattcttgatggcaaagtggttgctcacatacagtgatccataactagtgctttatttttgttgggaagtccaaatgatactcttttttgttgaattgaaaatggggccaattggtttggagccggttttttgtcataccaaaatttagtgccaaaatgttggtatgttttagtaccactagattgatagtatagaatttgtgtagtatgtgtaaaaattgatagcaaacaaaacatccatataacattattgaagttgccaaatttttttggtaggacaaagcactataaaaaaatcattttcccatacaatcaaaaccaattttcgcatgtggttgggtcgccgcctgctagggcagcgcacctgtccgcgaaaatccgccttctgcgggcggtaaattatgtcgcccacccgcggaaagaaaaattgctaaaaccctatctagctcccatccacaagtgacattaacactatagtctgctatcgctcgtcaccgtcgttgccacagaggccccgcagctcatactcatcgtcgtcgccctgcccccatgccttcaccacctccaccgttgccccttctcaccccggtagcagcggcagcgtggatccgcctgtcacggggatgctcgacagcagaacccattgcggcgccacccgacggcggatcccgcctagaggggagttagctgccgaatcctcgtgggagaggagttgagagagaagggatcgactacagtcaccgtcgccgagcttgagcatgccgtcagctgctcgggagggggaggcgagggagagaggaggtgagaaatgagagagagaagagggggggggggttgagagagataaggatgaggaagggaggaggtgagaagataaggatgagaacttataaaattttgaagatgtggaaaggaagagagaaagacggtcaaacttttcgtaggtagacctcttaatcggcctcttgagataatgtattttcgcatgcagtctttgaactgaccccgggggagcaactttgcatgttgacgtagtttcggcccgtcttcagcctagagggggtcttgtacagaaaaatcgattctgtagtagtgaaaatttattttaatccaaacaagcccacaatcttaataaacttttcaaccatatattatgtagagagaaacatgactagccaagtcggacatctcctaaatgttaaagtatatctacgaacaaccaatttgctcaccagcgtaatgtatgtcagcttgagcttcgaatgtttgtaatccatcccatccataatgtaaccattcgtggttgcaatggtggccatatcctttactccttccctccgaaaaaaattaaactctaggatcatgtttttttataggacggagggtgtagatgtaaaccttagttaagcaaatgtagagcgtttagataagtaaaaatattataatcaatactaaatcaaatttagtaagaaactagttaatcttttatcgtacacggtgtctttgtcactgacatgtggacccagggatcatcaggcccacatttcagtgataaaatcatgatcatcaagcccacatttcagtgataaagtcatgtgacttcgactatagaggatcctgatccgattagtttgcctctggcatagcgttggtattggttgaataatcagtttaagtcttatctctctctttcatcacatcagtatgtcttaacccgaaatcatacaaaataagtactgtatcaactttgccatatgaaaataacataatttcaaatattgttttcacctggaaagatgtgtaataaaaagttgcttttgtcaatggccttgggtaggttgtatcacccagagttacattctgcaatgtgacagtttgttttcttttaacacccaacgttttcgatgaccagagagagatagagtgagagagtcgttaaattgtggcccgtatattatccgtgaatcgttaaattgctgcagagctgaacaacgcaagaaaagacggaaacggtgccgccccactacgctacgctagtgctgttcctcctggtttttgtggacttgcagcaacgggcgtgcatttgtttatctgaattttctgattacatttcggcgtgcgctatggatggaggaggatcttcagatcaatccagcatccatcctttttctaaggccacgaattttcaggtttaatttcttctggatcatgttaagaggataattcagcgtcctaactttcagcttttctttctctgtgaggctacgaagccaacggtttggtaaagaaagaaaaaggcaaagtctgctgctgctcatcactgtccactaatagttaagttatggatcaaacatgatcagggttttgctgggggatttagccctgctaggcttcaatatgttgggcttgccgggcttcaatatggtagcaacaagaaagcagcctgtctggacaaagttgggagcctactcgtactgaacggcgattgttttttttaattgctcccactgatctatttatgttactatgtataactgatattggtactattcaatttcaaattttgacaacttctttttttccctaaaatatattataatacttgatatatattatgttatattaccaaagtgtgtatacgtagtgtttcatccatcatactctaatcattaaaaagcaatttattgttaaagtgagatgcatggtagcagttgacagtgtcattacaaaattataaagatggtcattatgctttaggctttatgcaaacaattaatatgctatattcggataactatatgggcggtcaaaatttggtacgctaatatttttaatgtattatttgggtaggtaaatgcaccccctttgattataaatatttttcatatttgattaaactttaaaatttataaccaccaataatttcataaatatttagtttaataaaaaaagacactaacacagacacgtctacctgtgttacatctataacgggttcaaccagcaattacaaccaattggttaaaacgtggccaatcatcattaacggttggaggccctttacatatgaggtggctgatgtctataattgcttctcaactggagccgttacagataagtgctaatccctaatggtttcaatattgcccgttacagatgagtgatttgtaacggctcgcttaggcccatcgcatataaccgttacagacgtgagaatctaagctagtgaaaataatataacagagttgccttgaaaagatatatattatactttataaacatgctttaatacaaaactactccctcagtctcaaaataagactcttttactttatattttgaactagatgagtgattattttaggacggagggaataataattaaagttttaaaggtttaatcaaatcttattttaaatgttaaatatttatgatagaagaaagcataacattcttacctaggacacatctgattttggaaataaataaatatttatgccccaaaattggctcatggttttcacaaaaccagtgtaaacaaccggctgtctagatcgtagccatgcagctagctggctgccagggatcagtagaagtactctgtgcagtgaacagggtgagccatgagcatcatcactgatggttctttttgagtggctaagcatgtcactgggatagcccatgatcattccggatgcaatgcatggctagcttcaaagtgaataaaactgatctagaaacatctggttcttcatagccagagaccaggctacaccatataagagaccactatgtactttcttggtcacaaaaatgatttgcatgctcaactaacatggtcctagatacgctctgctgggtccagagacaagtgtctagatcgtggagaggggaatttcagtttatgggtccgattccattgtaaggattgcccccaacacagttccatcacaatctagtgaatcttgggatttgatctaaggaggatgtctctactcggatgtagcacaacagtttgtttctgataagagatttgatttatagagtggactctgctcgggcatactgcaaacgttttattctgattagatagcttattcttttaggagaacacggaataaagggacatatatatatattctacgcacatcaccctcacacagatcaagcttagaaattgctagcaaaacccaattgcccattgtatagctcacatatatactatcatggaatagcttacacattgaacatgtgtagcactaaaattggtttgtgccaaacttaatggcgcgacttaaaattggagtaatattctatttagttcggactacatcgtcgaatgataagtacggtatcaagtttatcgaaggtgatgttttagaaaccatatgtattgggtgccacaagttattccgatttgtttcagtttttttttcctttcttatttttcctgcattcctagttagtgcagatcgaggacatataagtattttttttgcgaggatatatgtattttttaacttaataaaatctgcattatctcgttctattattcatcgtctgatcacatgcatgctagtttctgataagaatagtctttcttcaatatttgagggaggagaggggattgcatatatgcaagtgcagaatttctacaacaggatgagttgtaaagcgcataataattcaccgaaatataagaatgcacacatgcaagatagatcgagaattggatagatccaagaatgctcaggttggtatgtattaagtccattgttgtggtcgtcccagagcttagctattgtccttttaacaaattcttatccccttagctttatcatcaacttgcatgctctgcgtgtgcaagaggattccagaaaagaagaaaaaatgtgctagagtggttgtattaaggtagcagatttggtagtcctagctttgcttgatccattgctttaggccatgaaggcatataacggcagggataagcccagattagcaccacatgacatggaagtgaaaggcgtgtgctcaccaaccatgtaaccactcaaaatctcacccttttcttttgtccacaaagctttttctatatgcaactatagtcttaattaattaattcaacaggaggcatcttattaccatttgaaagctagatatatgaccaggaggcatgcatgtacacatatatagctagctagctatttgatttgaacaatataaaggccttgataagtgctactaatccataacaacactagtagtatccgggttctcaatcacattgtggaccattttggcatatcattttgtgcgctcaatcttgataagtactccctccgtcccataatataagagattttaagtttttgtttgcaacgtttgaccactcgtcttattcattttttttaaaattattatttattttatttgtgatttactgtattatctacagtactttaagcacaacttttcgttttttatattaaaaaaaaattaaataagacgagtggtcaaacattgcaatcaaaaactcaaaattctgggacagagggagtagttatttgtgcctttaatctgacatgctagaagggggtagggggagtgtattaattatatagtgtaactcattatctgattgcactaagaacgatctagctttgcttggcttgatagctgaactgctgaagtgcatttgccatggcagccatgcaaatccatactgttatactagtttttattatctcatactttccaaaagaaaaaaaaaggcctttactctccagcttttgttttttttagaaaaaaaatgcaggcatttgtcgttctttggccccacttgtcccaggttaatgtttgggatttaaagttttaacctcatttccactaggacattacgtgttgctttgtatccatcgttagcagagtagagagtgtgaatcaagcgcacatgggttaatccagaatttcagatccatcatccttgtgcaaaggaaaggatatatgatttcaacatacacaagagctagtgtgaccagtatagcagagtgtgcacccacatgggcatatatattaggaaaaagtacgaattaccatcttccccgaactatcgcggtcgatcgaattaccccactgaactcgaaaaccagatatcgctcaccataaactttcaataccggacaaaagcaccccctaactcaacatcgcagtggttttggtcctaagtggcagtccagtcagcaatttttattttaaaaatggtggggcccacctgtcatagtctctcctctctatcccctctcacctctctatctctctcgtctatcgctcgctcaccgctgagaatgagtggagcacgcgccggcggtggttggcccgacgcgatgggacggctacagaagccggcgaccgtggaggcgaaggcagcgcggtcgaggactagcggttgcgagtctcctcaaggcggaggccgaggatgaggcgtgccggcggtggctagcccatcaggctcaccaggcctcgtcctcctgtggttaggggagtagcggctgacaccggacttacgcaagaaggcagccatggcggggaagaggggcgaagcggcggggcggctgcggaggccggtgaccacggaggcaagggaggcgcggtcggcagcaaggccgaggcagctcggggtcgcgggtgagcgccgtcggcgggtgaggcagctcgaggtcgcgggtgagcagcaaagagaagaggagacgagtggttcaccgtcgccattgaggtcactgttcggcactgaggcggaagcggtggcgctgctaatgctcccttccctggagacggcggtgaggcctccccccctctctctctctctcctccttctccactcaggcaggcggtgagcgacaaatcctcctcggggaacgcatggcgcgcgcggtgggcagggcgaggtgggcggcaccgaggacattggaggcggcaggctatcgtgacgagtttgccgcccatccccgtcgtccccgtgcccgattgttcctctgccgcctccactccaatctcctctccttcccgatgtcctcgccggcggctctaccccactggtcccgtcacgacgtgccctccgcaatcttgccgtgcaccagagggaagaaagagagaagagagattttagcagtactccgactgccgccgcatcctctgcgtgctcgctgcgccgcccttgtctccacctccatgccgctgccggcacgtgccgccctcgcttccacctccttgccgctgccggtagtgccgctctactgtgtcgctccgtctccccctgccgccgtgcgctttagaaggaggaagaagagcgccgcgtcgtgccgtctccccaaccaccacccgctttagaaggggatggagaagagagaggtggtggggattctaacgagtgggaactagcgattttttttttaaaaaaaactgatgattggactgtcacgtgtgctatttgaatcctaaaccgcttgcaacagtgagcgatatctggttttcaagtttagttggtaattcggtcgatcgcgatagattaggggggtaattcgtactttcctttatattatggattgagaacaagaggatagcttattcttcaacgcatccttgtgacaatctcctgcactcccagataaacagatcatgcatgagcatatatatcgatcttgttagaagatatagctagctttgattgtcagtcaaagatatggctccctttttatcaccatcacagtcaggggcaacatggctagaggtcacttcactatcaacggtcatggatcgagtatactgatccacttttaaatgccgtatccttacatactccctccgctccaaaagtctgtctaaatttgtagtactaaatgtgttatatccatccaaaaatctttatatatttggatggatatactacatacattttgtcctttaaaacataataactaataataactagaaaggtggcccgcgcaacgcgcgggcatccatattatttaaatataagattatattttgttcgtaagatatatcttataaattaagggcaactcaaggtcacacgtatctaaaaaattattcatcgaaattttaatagagaatggattaaggtcacatgacatttgaatcaactgaataataaaatgctgctaggtaattatcatctctatcatttatacacatagtgtgacagtgtgaaatatgtctataagctaaacttgtgagagtaaggaaatcaaagttctttggattgaatcgtctcattaaggcacatgatatagtgacaaccaaattaaggatagatcaaatgtttgatttttttttcctatgtagaagttcaacaaattcattttttttcacagttttgaagaaatttaatggtatgttaggacacatattactcacacaattcattggcttggtaatgcaaaatattagcaattgtttcaagataatttctttctttttttcatgcttgattgttttctttgaaacaacatttgtttgaaataagatcaaaattgtataaatttgattagtatgtgtagtgtacttatacatttgaagtgttctatagtaaagtttgcttttgtcatataatccaattgtatcaaataatattgttttatcatgtaaattataatcaataagttcaatatgattcatatcatcataattaatgaaacttagttaaaaaatatgactacctaaaaattattgttcgcttattttgtttttttctccttacttaatggtgcctttgtcattactctgaatcaaagaaaaagtggcacatcataccatggctcatatatagctataagctattactctatgtccactcattgctcagtagtgtccttcactcaccgatcatcttgtgatagtcatatagacaatatttatcaatttgtttagtttacataaagggcaacaccacattgctcctttctcatgcatggtcatagagtccaaaatgatttttccaaataatgctctgcattaacacatgtatctagaacattaactacataacatatacgtgtggaacacatggtagttttcgttctagattcacccttatttacatacatgtaaagagtgtaatgacaatcaatagtgacacctattaacaattgttatatacctaattagaatatgtattaattatattcatctcatgactctgactatctttgatcaccttgagtagtagaaaaaaataattgtactacgggtttaggatgggtgaaactaatggattatttgatttttatgatatctatttgagggtataaatgaatgaattaacttatacaaatttgaaatgttgttctggaaaaagcgctttaagaaaaaaaatagaattgttttcccgaaacacatcttttaggaacatgggaaacatgcaaatgaaaatacaaaatctagatcgaaaaggaaaaacagggagtaagactcccttatattatataagtacaattaggtaatcaacccttcgttttagcttacatagagttgcatgttgcaaaaacttaacccaatggacttcattattttccttctgcaatagtcgttcaatttgttttcactttattaacaaaacaatcattatccattgatgatttgatgtgtcctcaccttggcaaactggtacggtgtggtgatcatgatcaccttacttgcctcaatctggtgcaccgcattgtcgtggcgcacgcatgcatgtgttgtgtagaggaacggactgatgtttttggaggatttttttagaaaaatagatctgatggtttattatctatcaatttaaatgaaaattgatagtcaaatattttagcagcaatatagtctcaaatattgggcccacttgttggtttaaatataaattagtcgatatagattataaattgttaatttaataggtataaaatataatggtgtaaaattcaaacaattttatatttgaaaaaaaaattaatccaccagttcaatttaaaaaattatcttatattattaatttaataggttaaaaatacaattttttaaactttacttttaaacattatagagctaatttttagttatatttatttgtaaagagttaatttatgatgatatatatagtctatgtttgtaaaattataattattttaaatttattatcactaatataactgttttatcacatgtacatggagaaagttggaaaggagagagaagcagatgtatttttttaattgatgtatataatttgtgtttttatgttttttttgctttaatcttcttttattgatagtgcgttacttacgtactttgtattttgggggttttttttttacgatcagtactttcgtgaacgggagattataagatggaagtttttttaaatagatctaatctaacaataaataacagatcaggtgttttgcttgtgttttgcttttcttttagaatttataggaatttctttaatttattaagtgccacatgacagcttgagatcatttgtataaaatttaataaactttttatatataatagataatagattgataaatacataattgaaatatagaatgataaagagtgttgattggatatcttatatacgatctagtttaaccgcatatatttatagagcgacatattttatttttaaatagtagaaaatcctactttaaaggccatcaatttttttaatgacactacgtacgtatgttttttctttactttatcgcttttatccggtgaagagtagataattaagcttcatcgtttttatttcagatgtatgtacgtactgaatactttcctctttcttttccgtcccacaggaggaaactattactttttaaaataatacatctaatcgaatggtccataatatcgggtccaccaattttaatgaaaatcgatggtaagattaaatagtgccacatgacgacctaagagtgtttataggagtgccacatggcggcttaacagtgtttgtaggaagtttaatggacttttagtatacaatagatagatagatagatttctgactataaatttagacatgggttattgaggttcatagctagatttttttttaagatggcttgagtatatgcaacgagaaacaaaggcatgcatacatgtcatcatttggttaattagactattgcaacttgcaatagctacctacctgatgtacgtcgaagcaagtacaagttgcatgcaagctttattgtctgtgtctgtctttaagaaaattaaagcagggagactctaggataagaaagaagaataagctgttcaaaatagaccaactgttcatgatgcttttcaccttcctttcgttaattagaagcatataatcccagagatttcagcgtcaaggggaaaccatttgtagcttggtagcacaggttatataactaaagccaccaaccaatcatttgggctaagatcgagcgatactgttacggtgctgttgcatttacttttgactaattaatctgcgagctaccaggccaaatggatcagccccaatgattaacaacgctcatggattggaactaattacacagattcatcccgatctgcaggtaacaaaaatatgcgaacctttctgcagccaatctatgcaatggtacagaagtgtaaacagataattggtggcttgcagagaagatgaatctgaattatataagcaggttgggtgcttgttgttggtggccatgtgagtatcagtatgtgacccaggatgactcggatgacacagctctgctcgccgaccgatatgaaaatgacggtgatcgactgcgactgccaccagttggcggcggccggacgagaccccccgcgtcgttcctcgtcgtcctccaccttcggctccctgcttaaactgcttcgccgacctgttagactatttggcaatgtacctgtgattggctcccttccctaaacctttcgatcctgtttctataaatcggttgagcgttctatagaagattgatgcctatttgtacagctaccatgtatcaacatgcatgattatcgcattgagctgtatcatccaacattccctgctccggcggacggccctgctcgccaagccctggctctgttggtctctgcctctgtctcaaacagatgttgttcgagccattgactggatggctaaaggagaacagagagaaggaaggatgataggacgacaggtgggccctgtaaattcattattattatttttctgattgggatgttatgttggcaccacgtgtaccagtttaagtcaagacaatccataagggataatttatccggtacgaaaagcttagggtgtaaaatatttggttttgtattttccgggggtaaatcggattggcacaatagttcagcagtgtaattcaaacttttttttaagttgttctactattttttactctctatcccaatataactgcaacatagtaaacataggctctatttagttcacaccaaacttcccccaaactcccaacttttcatcacatcacatccaaaacttttctacacacataaactccaaactttttttttccaaactaccaactttccccaatttcaggaactaaacacagtcatagtatagatgtgacatttgtactaggttgcatttatattgggacggaatgagttttaaagatagtcaattcgaatatgctaccacttggtgacaatgatgcatataaaaccatctatgtaccctctatttaggggggtttaagattctgagaattaaccaatgatagtcacttagtaatcaatttagcgaagctggaaaatgcctttagtttatttttattccacagttgctgattcttcaaatctaaattgtttggagtagctgtagagatttactaatacacattgtgctgcttgaatctttttcaaacatgaccatagaatagatgataaataatcaagatctctctttgccggagcaatggagggaccgggagaacacataggtgagagtgagtcgatcaaacgaaggacatggggtctgtttacttcttgccctgaaaatcttgcctgactcagacgcaattctccgaaatttaattttgtgtgcctgcagatcgacctgactgagcgagagccctcagtcaaccaaacaaggccatgatcccccgccgctcgtgggcgcacgcacaggccatgtgacccatgttagtaggccgccgcgcatcgatctggacgcgcgactgcacctacgacgttcgcctgaatgcaagcgtgatgcatgcacccgtagacgttgctattggagatatatcctatatgcagtgtagtacaagtactatggtggtagtacgtactatattttgctgcttttgataacagaggattatgagaaaaaaaataccagtaagaaaaggaaaagaacatacgtgttcccagtagattttggttgccaacagtgcgatgacatagtaattaccggcgaattaaccatgacaagctgacgcccaatggatgaagtggccgagctagcactgcagattaaacaacaaattaacctgcaatggccaactaatttagctagatagccaagtttccgtgtcagccatgtgcgcccgaggagggctaccttgtgcagctgccggaggatcgatcaatgcagacatgcgcacattcatgcatgcagctgcaggagcatgcaacggcggctggtcttaattaatttgctgaattgagctgatctgcatgcatgaacgcgtgaaggcacaggcagagggccggccgttggtcgcaatgcagaggcagctaggcgcgcgtacagtgatctgcctcgataccgcgtcgatccattcatcggctggtccgtcgtccgtccgtccctgtcgcccacgccggagcctagagatctgtcggcctttgggcagccacccaacagtcaagcgtcgtgcgtggtggctggaccattttctcgtagtaccaaactgcgtaccaaaccgatcgatcattaactaccttcaagagtactctctttactgtactactcataatcggaggccgtatgtcgactaaatactgaggtggagaaaataagaaaggagagagaggtgaagcaggctgtaaacttatagccggtttggatgtaagaaccaaaatattctctgagagagacaagtgcgccatatattaattctaagagtaaattgcgttgacggtacaagaacttggcgggtgggtgcgattaagtacaagaacttgaaagttggacatatcgatgcaacaacttgactaggtggatgcgcttgtggttcaaaaggctgccacataaggttttttgccacgtcagctttccaccttggacaaagtctacgtgagggctctttacacatcataataattttgcacaaatcccctgctgtttttcccatttgaggacattaattaatcaccatgagggataggacctcaaatatgtagcttccggctgtagcggtcggtgggactcaaagccggcgtgcggcagcagtcgtcgggactcaaagctggtggcggtggtcctcgggacttgaaaccggcgtgggtcagcggtcgatgggacacgaagcccggccacggcagccgtcaggactcaaagccagtgcagggggttgttagatctcgaagccaccgtcgctgcccatggccgtagtaggttcccttcaactgatgagaatctgaaccttccattttttttgtgctgtaattctatgggattcttcgtccatagtggaatcagcatcatgataactgcatctggtgtaccacagtgagaggactctttgactctttgttgctgtcaaagcagtatgtgagtgaataatggcaatgtaggtgcttttgtaaatactctggaagtgcttttgttatataagggatacatgaattttttagctacttaaatgaatggtgaacacgatgtagcaaatactgcttgtattctctcttttggaattgtatgtccaagtgataacctgaattttgctaacattgctttgaccagtagcaatttctgtacagacatatgtggtatgttttcttgtccatacacaaatcctggcagaaatgtgctcctaatattctaaaatgttttaccataaatagctgttgtataaacaggaaaaacaagagatgttcctgaattggaacttagagcaatgtaacagagcacagtccaaaatgaaacatgtctaattggttggcaaaaaataaacagaacttctggctaaattataagctaatcctttttatcaagttgaatatattgttcactttttctttgtctcctcccttttctttaactctgatttcttactgggttgcgtatttctcttccttggctcagcttttctcttccgaattgttgccctgccctctttggttgctgtcgttattggcggctgcactgcagcctttcggtttgtggcaataaaatttgattccggcagtggaccaaggtcaactctagaagagttgtttataatgttctgcagaaagtacattgaaattgtgaaaactgttttataaagctataaaatggaaaattggcaaagcattgtctaacctctctcgtcaaggaagacaatatggtattttgtaactgatcatagctaggttggttggttaaaatgctagtctgactgttcagaacttcaggcatgacctgcagaggcataaaatcttcattaccatgttgttctgaaggagcagcactggcttgctgctttcctgcttttatatcttccttcctcttcttgcagcccttcttgttgtgattaactccttgacagtagctacaatgcatttgcacaccatgcttggatatttttgttcctccttcaagttcatgtggctgctttcttctacatctcctaggccttccaactttcttctcataaatcggtgggttcacctttggtccattcatcttcacccaagatgccttgtccctaacaggcattatgttaaatccataggcctccatgtatttttcagttgaataatagaaggataccacatcttcaggttttgtcctctcactcctcaagcaagaaattgcatggttgcatggtatccctgtcaattgccatctcctacattcatagtgttttgccatgaggtcaacaatatattgatgttctttatcagttacttggaacacccctttaccagcaggtaatatatagcatgtatttgacaattctacatttttatcaacctttttctgtatttttatcaacctttttctgtatttttgggcatatgtttcccgaccacttctttacacactcctcttgtttggtatgtatcctattcatgatttggctcctaatcttttcaagcatggataaaattggaagctctctggcttcaagaatgtacttgttgaatacctcggaattgttgttgagtagaatatcacacttggggaaatcactaaagaatgctctgcaccactggttgggtggtatttcctccaggtaggcataagcatctttgctaagagacttcatcttttccatgttaaattcccactctggtatagtacttgaccttgcaattgcccacaactgattctttagagtctcccctttgtatagaacatgaaaattttggtacaaatgcctaacgcaaaatctttgttctgaatctgagaatttatccctcacagctggtaccaatccctacaaaatttaaaaaggaagggtgagtaccaatccattgaacagtaaataattcaaatattttaaaaatgacaagttgaacaaaatcaaatatgatggaataaagtcataccttctgcctgtctgtcatgatagtataaggtccagtgttgtcgataaggagatcttctttcaaagtattcaaaaaccaaccccaactagctctactctccacctccacaacagccatggctataggatagatgcagtcattaggattaatgcctactgcagtcaacaaatgtcccccaaacttggttttgatgtggcatccatcaaggaatatgataggtctacaaccacttaggaatcctcttttgcaagcatcaaatgacacatagcatgtatggaagcacatattttgtaggttcaagaaaaatttactacctggatttgaagtcctaagctcttgtccataatcccacaacatatcgtattgtgctatttcatcaccatatatcgctttcatagctagtttccttgctcgacctaatttatgtcttgatggtgttacatttagttccttttgaactaccttagagaaactcttgagtgtcatcctatcattatccctaaagccttcaacatatctatttgcaagatacctggcagttacataattcaactcccactccttctgacatttgtggccatctatatatctttttaccataagacaattcgtcctgctgtcctcagctacatccaacatccatgagcactcttctttacatttagcctcaatttttttcttggtatttcttgtaaatttaattgcgactctattcttcacactgtattgttttattgcttgcctaacttcaatagcatcagagaacaacatcccaaccttaaagattggtgactccatgtccacagcagccacaaagttcttaaatttcaacttaatgccttcattatcttcatcagatgactctggaagcaacaagccttcatcatcagagtcatgttcaggaagcttaacaaccatcttgctgctagccacttcttctatctcaacacagttgtcaaaaacatcatcatcccccttgtcagcctcatactcactgtcaacaaagtcaggatctacatcgatgtcatcgtcatcgtcaatgtcatgaccttctataggtttaacatctgaatcttttcgattcattgtagacctctcacggctagccacgataggtatagaagcaggggtagaaagagcaacatcatcccaattgatggattcaagaatgtcttggtgatccacatacaaaagcaatgtcctaaaccttggtgctaatacaaccatgctgttggtgcttgcatcagagttaattctccttagcccatcactgaatgtcttcccaggcaataaccaataaaccttggccttctcagacatatcatatcctagttgctcaataaaatcatcaagccataatgaagaccatgtgtcactgtcacaataatcaaaccaatccaccttttcatccaaataaattctgctactaccaactccagagaaaaaaccaccatgatgtagttcagttgtgaactgttcatcctcgcagcctacatcataagacaaaccattctgatgtgtcaaatcgtaaataattatctgatctattatacacaaaaagattatacagagtacatattcatgcttagagcaaaaattatttctaatcacaaactattcgtgaaggattacttatatgacacctagccttataaaacatcgagaacaaaacgaagaaattaatcaggactcgacacgtctcactgcaacagtgttaaaactaaccactacttttagagcaagttttaatagtacggggcaattgcatcggtgcactcactttcgaagtcaattgttgttttacccctactttttagggcttgcagttttaccctcacattttaaatctaaatcagccatgtacccccatttctaacggccgattttaacggtgctggatgttaaaaaaaaaggacatttatgtcctgggtggttattgtatttttgccctatttttttctgttttgcgtgattttgctcctaatttgtgatttgtgatttcagcttgtgtgatgagcattttgaccaaatatgagcatctcttgtgatgttttttactttaacaaaaaagaatcacaaatataggcaattacaaaatttaaacaaaaatcccaaatactagccttcatggtcaatgctctagaccttgtgaggtgctgagagatgagagtcatgttaggcatcttggagtaagtatcaaacagattcgtagctcccgagcttggcagcgcaaggcttgctggatccagcggacgaagagccgctcccttccaggcactaacctcaggaaatacatgtagaaatggcagtgattcagaggcgcgttgtagctgcggagcgtgaaaacgcgatccacgaagctgatgaacctgtccaactcctgataaccccaccaatccatggagaggcccggtgcttccttccagtggtggagccaacgacgggcgagcacgcacgtcgccaccgcctcgtgcgccgggagcagcaacagcacgaggcagagcacctcgtccggcagggcgctgatgcggtctggcggcacctcgtgcatgtgggtggaagcgcggcgccgcctcgcgcgcgcccgcggccgccgccgccgcctcctccgcgcaaggaccaatccggtgggagaggagagctggttggatgtttatcgattccctcgaggatagaacggtaaattgatatttcataaatcatttttattatttctatttgtttttctaaaaagatgtgggaccaacctaacggtgttaaaactgagggcgttttaaaaaaataggggtaaaattacaaaccctaaaagtgagggtaaaacaacagttgaggttaaaagtaggggcatggatgcaattgtccctaatagtatagccaaatgttaaatctaaatcatctatagctattctaacagtccattcatacaataattaagtataaaaatatactacaccattaatacctggtctcaggtattatacccatatactccctcttcgtattttaatgtatgacgccgttaactttttaaccaatatgtatgacgccgttaactttttaaccaatatttaaccattcgtcttatttaatttttttatacaaataaaaaatacttatgtcatgcttaaaaatacttatgtcatgcttaaagaacatgatttaatttttttatgcaaataaaaaaaatacttatgtcctacttaaagaacattcgatgataaatcaaatcacaataaaataaaatgataattacataaatttttttaaggaagacaaaagatcaaacgtttgttaaaaagtcaacggcgtcatacattaaaatacggagggagtaatatgttggacataacgtcttagaatccatgttacagctagatacaaatagcttgctttcttctctcttctcttttcatcgcatatcatattcggcttatagcctgctatgtacctgctctaactaatgtttttgatctagtacatgttgttgtgtacagtaatcaacctagtatacatgcacgcatgcatgaagataatttgatacgcatcgcacaatatgtaattaactagtaggattaattggtaatcacacgcgttctaatttgcatgttaattggaataccttgctgcgtggtggagagatcgtggtaggagcagaaccgggagaagatctcggcgagggtggcgcacttcctctcggcgccaccgccgccgacgccggcgtcggaagggacgacgacggcgtagaagaatccgggcatctcgaagacgaagacgcgggcgtcgaggcgcgcgacgagggcgtcccgggacagcatccagtggtggtcgccgacagcgcaggaggccatggagaccgggtggccctcgacgcgggccatgcgcaccgacaggccgccggagcggaacagccgcgccgcctcgaagcggtcgccgcacagcacgaagaggctgaccggcggcgagccctcagcggcgagcagctgcgtctccctccccatccggtactcctgcgtccgcccgactcgcagcggcggccgccctcctcctcctcctcctccctcccgagtcgccatctccgatcgatcaacacttaatgctgcttgatcacacaaattaagctactagattcaatttcactctgtactgcaaaagaagaagaggaagaagaagaagctgcaggcacgcaaagagcatgtccatacggcaaatatactggtcctttttattagacagtgatagatcgttggctcgtggctagatagatatttgttgcccggatatttttccatgcagctaatattattcgctaggttaaacaacggcacgtgttaaggttaatgaccatgtatgtatgcaccactcactcaatgctagctaccagttaaactatggtacgtagtgcttaccacaacgagagccggatcggagagaaaagagaagggaattacgttagctagagagggatcatgttggagtatatgcgaggttgtacgagctgggagaagacatgggggtggccactcttaaataagagggaggtagtggtagtagtagatagccatggtcgccgaccgaggaagaagaagcagtggggggtcgcgaaacggaggccgcgcccgcgcgggagaagagagcgtattgcattgcattgcatgcatgcagtgcacgcgccacgcacgcgggacacctgcgcgccacatcgagcggggggtaggggcccaggcgtggggcccgcaggcgctacacgcggcccccaccgcagcgcgacgtggcccgacgacggccacgcgtcccgcagcgctggaccccttgcatgccgccgcctcgcctgcgccgcgaatccgcgaccgcttcgcgcgcgcgcgcgcgcgcgcgcgccgagccgagccgttcgagttcgcgcgctgcgcgccggtcgaatcgaataccggcggcattggccggcctaatcaggggcttagagcaagcataatagacgtctctgtaagaaagtagacgagagaggagagaaaagtgaggagaaacggtctatgaatttgtctataaatttacagctggtttagaaaaaaaaccaaaaaataaatctatgaaaagcagatggacaatatatttattatagtgaaaagctaacaactatatgagtaggttaaaaggtagactataaaaatctatataatcagtagctagctgtattattagctatgctcatagggcctgtctagggaccttctagttgtgtagtatcttttagaattaaaaactccctacacagtttagcttttagtttagattctaagaatttatagttataaaatctaaaaaacaaagtagaagtcataagctagaaaacctagctagcttttttaagattatcagtagctagttatcaattagttgtttcttagaattttaaactcttctaaacaggctgttagctacacctggtaagcctacctaccgaagacaattatata

SEQ ID NO: 19 - DNA, 39530 bp, Oryza sativa
gctctcagcgggggcagcggcaacgacggagcacgtcgccgacgagatgctacgccgtaatgtttttttttctttttcatcgcctgatttggatttcgcatcgctcccgtcaggatgcactacacggaatcgcgttctttagcctcggtttcaatgacaggacaccgttgatctcacgcgaacgtgccgttgcgttttactgtgtgtactacaaggaatcgaatcttgcaaggtacagtatgatatccgtgacgggcaacgacgataccttgacaggcaggctcggtgcatcgatttctttcttcttttttttgatttgaatgttccgggcggagttggaaacagaggcgcgagagtggaggaattccccgccaaaaaatatctgagagattcgtagcagtaggtacagcattgcgaccgcaatagtgaagtaagatgctctttataaaatatgtacatctcagcaataaactagattaatatgtacatcttagcaatagactagattaatagtaaatcacctcaatattatgtctacatgggtatctatagctctatcatgtattgtctcatttttctctatagactatttttaagttagtagataattttgctctttctttttatttaatatcttcgaagtagaaaaatatgctgacatggatctcttgtagagagcttatagataactattgtccctagtgcacatggctggcgggaaaccgcgtagcctcagcgtccgcgccagcgacgactctcgtactctctctctctctctcctctcgtctacaaaaaggatgggcctcattggccccacccacgtcgccctctggatgagggtggcattgaatgcgtgtgttggtgggccccaccaccacgcaggaggtgggcccacctgccagtgtcacaggggctcagtacgcagaaagagagagagagatcaggtaaaaatggaatggaaaggagagccgccaaggcggccacaggtgaaacgccgacgacgaccacgaccaggcgtttctctgactagtgagtgatacactgatactcctacaattaactgtggtgctttggttggttgctttggcacgtggtaactggcaatgtggaacgcgcgctgtccgtcactagcgattcttgtagagtgtagtacaactatctcatcatattttcacctgatctaagggcccgatcgagagagtgagggtgggggaataatctctctggcacggaaaacggagcaggccattagcacatgattaattaagtattagctatttttttaaattggatcaatatgaattttttaagcaacttttatatataattttttttacaaaaaaaacacaccgtttaacagcttaggaagcgtgtgcgtgaaagacgaggtggtggggacgtttgcccgtgggaacgagcgcacccttaaagccgtgaggcgtcatcatcaatccggtggcaagttgggcttgtggcactgtccggctgcacactccagtagcagtagacggtactccttaggccattcgccgtgcgtcaccgtggcgttctacaccgactcgctaaacttgggcatgtgttcttcgtcagttttggcaggcatgtgtcaaagcgaggcgaggaactggcgaagaacggtgcgcacaacacgttgggagagggcgtgggcctatgcttcgtcgctcccaacgcaacggtggctggatctggtgacgtcgatggcgtgggcatcggcggcctggggagaaaggaagatggcggtagcggtggaagaggaggcggtgaaggaagaggcgacatcatttataggtcctaaccttgttctcgttcccgtttcgtaccgtcaacctcgtcgtcatcctccgctccaagccaccttggccagtccgcagcagcagcttgacgatggcctcctcgagtcctccgcctcccacgcttaccactagatccgtgccgtcccattctgtgccaccccgctccatgccgctgagcggccgtcccgctcgtagtctctgtctctccgagctccacgccggcgcgccagccagggtaggatggacgccggtcgtaggtcaggggaggagtcctcggtcgccggtagggggaggagtcaggacattagccggtgaaggaggggagggagccaaagtcgctggctcgttgtcgggagtgagtggaggagaggtcgagaggagataaggggggagagagacgggggagggaagaatttataattttgttttggagtctagggtctttggtataaattttgaacctctacacaaaatgaaaaaagtatgagaactaaatcaaataagtggtagggactgaaatataaatttaaagccacacctaaagccacttgcaatatggagaactcaaattttggacctgtgaccctagcctatatggcagtctgaggctcctcgtttgactgggacacaatgtgaatggctttagtagtttacatcttttgattcttttagttaaaaacttattttatatatagagctatgagaaaacttacaccttctgtgctatgagaaactaacttctgaggattaatctggacaaatgtttatctaaatttatcttaaaaatagttttttttaactattctaggaatcaacgttttttcacggtgcaaaggtaatgataccgagatgtcttggtgtcaatatcgccaataacgttagagttgacaccctagccccatgtgatagctaaatcattgacgtttagatgttggacttcgatatcagaatcactagcgtcgggaaaaaaggtctaattttaaatatgtgcttttaagaatctatttacaaaataatttttaaaaggctcgaaacataaatagaatccgacagtagtagggtaagtctggttgcttggctgggcttggcgttccatgacttctatccttgctgctacacaggaaaaatatacagtgcaaaccacatgacatatacgcagtagaaacctgaaaactgaaaactatagttacataaaaaaatagacgtattcgtccacgtcattatgagtaaaattttgacccctaatgatggtagttcttgatgcaaaggtattcatacgtctattttttaatttgatggtagtttttgggtgtctactatatacgtaatttccggtgtatatttatactactctatgctgaatgctgaagtaaatgtgaaatggcaagcacaccagccactggacaaggaggtagggtcggcctcatctgcaactgttcaccggtcaccaccaccgttgggtaagtagcagcagcgtgtagtgggcaagtctcaaatacagtgatccttggtggccggtacaacattcttgatggcaaagtggttgctcacatacagtgatccataactagtgctttatttttgttgggaagtccaaatgatactcttttttgttgaattgaaaatggggccaattggtttggagccggtttttgtcataccaaaatttagtgccaaaatgttggtatgttttagtaccactagattgatagtatagaatttgtgtagtatgtgtaaaaattgatagcaaacaaaacatccatataacattattgaagttgccaaatttttggtaggacaaagcactataaaaaatcattttcccatacaatcaaaaccaattttcgcatgtggttgggtcgccgcctgctagggcagcgcacctgtccgcgaaaatccgccttctgcgggcggtaaattatgtcgcccacccgcggaaagaaaaattgctaaaaccctatctagctcccatccacaagtgacattaacactatagtctgctatcgctcgtcaccgtcgttgccacagaggccccgcagctcatactcatcgtcgtcgccctgcccccatgccttcaccacctccaccgttgccccttctcaccccggtagcagcggcagcgtggatccgcctgtcacggggatgctcgacagcagaacccattgcggcgccacccgacggcggatcccgcctagaggggagttagctgccgaatcctcgtgggagaggagttgagagagaagggatcgactacagtcaccgtcgccgagcttgagcatgccgtcagctgctcgggagggggaggcgagggagagaggaggtgagaaatgagagagagaagagggggggggttgagagagataaggatgaggaagggaggaggtgagaagataaggatgagaacttataaaattttgaagatgtggaaaggaagagagaaggacggtcaaacttttcgtaggtagacctcttaatcggcctcttgagataatgtattttcgcatgcagtctttgaactgaccccgggggagcaactttgcatgttgacgtagtttcggcccgtcttcagcctagagggggtcttgtacagaaaaatcgattctgtagtagtgaaaatttattttaatccaaacaagcccacaatcttaataaacttttcaaccatatattatgtagagagaaacatgactagccaagtcggacatctcctaaatgttaaagtatatctacgaacaaccaatttgctcaccagcgtaatgtatgtcagcttgagcttcgaatgtttgtaatccatcccatccataatgtaaccattcgtggttgcaatggtggccatatcctttactccttccctccgaaaaaaattaaactctaggatcatgtttttttataggacggagggtgtagatgtaaaccttagttaagcaaatgtagagcgtttagataagtaaaaatattataatcaatactaaatcaaatttagtaagaaactagttaatcttttatcgtacacggtgtctttgtcactgacatgtggacccagggatcatcaggcccacatttcagtgataaaatcatgatcatcaagcccacatttcagtgataaagtcatgtgacttcgactatagaggatcctgatccgattagtttgcctctggcatagcgttggtattggttgaataatcagtttaagtcttatctctctctttcatcacatcagtatgtcttaacccgaaatcatacaaaataagtactgtatcaactttgccatatgaaaataacataatttcaaatattgttttcacctggaaagatgtgtaataaaaagttgcttttgtcaatggccttgggtaggttgtatcacccagagttacattctgcaatgtgacagtttgttttcttttaacacccaacgttttcgatgaccagagagagatagagtgagagagtcgttaaattgtggcccgtatattatccgtgaatcgttaaattgctgcagagctgaacaacgcaagaaaagacggaaacggtgccgccccactacgctacgctagtgctgttcctcctggtttttgtggacttgcagcaacgggcgtgcatttgtttatctgaattttctgattacatttcggcgtgcgctatggatggaggaggatcttcagatcaatccagcatccatcctttttctaaggccacgaattttcaggtttaatttcttctggatcatgttaagaggataattcagcgtcctaactttcagcttttctttctctgtgaggctacgaagccaacggtttggtaaagaaagaaaaaggcaaagtctgctgctgctcatcactgtccactaatagttaagttatggatcaaacatgatcagggttttgctgggggatttagccctgctaggcttcaatatgttgggcttgccgggcttcaatatggtagcaacaagaaagcagcctgtctggacaaagttgggagcctactcgtactgaacggcgattgttttttttaattgctcccactgatctatttatgttactatgtataactgatattggtactattcaatttcaaattttgacaacttctttttttccctaaaatatattataatacttgatatatattatgttatattaccaaagtgtgtatacgtagtgtttcatccatcatactctaatcattaaaaagcaatttattgttaaagtgagatgcatggtagcagttgacagtgtcattacaaaattataaagatggtcattatgctttaggctttatgcaaacaattaatatgctatattcggataactatatgggcggtcaaaatttggtacgctaatatttttaatgtattatttgggtaggtaaatgcaccccctttgattataaatatttttcatatttgattaaactttaaaatttataaccaccaataatttcataaatatttagtttaataaaaaaagacactaacacagacacgtctacctgtgttacatctataacgggttcaaccagcaattacaaccaattggttaaaacgtggccaatcatcattaacggttggaggccctttacatatgaggtggctgatgtctataattgcttctcaactggagccgttacagataagtgctaatccctaatggtttcaatattgcccgttacagatgagtgatttgtaacggctcgcttaggcccatcgcatataaccgttacagacgtgagaatctaagctagtgaaaataatataacagagttgccttgaaaagatatatattatactttataaacatgctttaatacaaaactactccctcagtctcaaaataagactcttttactttatattttgaactagatgagtgattattttaggacggagggaataataattaaagttttaaaggtttaatcaaatcttattttaaatgttaaatatttatgatagaagaaagcataacattcttacctaggacacatctgattttggaaataaataaatatttatgccccaaaattggctcatggttttcacaaaaccagtgtaaacaaccggctgtctagatcgtagccatgcagctagctggctgccagggatcagtagaagtactctgtgcagtgaacagggtgagccatgagcatcatcactgatggttctttttgagtggctaagcatgtcactgggatagcccatgatcattccggatgcaatgcatggctagcttcaaagtgaataaaactgatctagaaacatctggttcttcatagccagagaccaggctacaccatataagagaccactatgtactttcttggtcacaaaaatgatttgcatgctcaactaacatggtcctagatacgctctgctgggtccagagacaagtgtctagatcgtggagaggggaatttcagtttatgggtccgattccattgtaaggattgcccccaacacagttccatcacaatctagtgaatcttgggatttgatctaaggaggatgtctctactcggatgtagcacaacagtttgtttctgataagagatttgatttatagagtggactctgctcgggcatactgcaaacgttttattctgattagatagcttattcttttaggagaacacggaataaagggacatatatatattctacgcacatcaccctcacacagatcaagcttagaaattgctagcaaaacccaattgcccattgtatagctcacatatatactatcatggaatagcttacacattgaacatgtgtagcactaaaattggtttgtgccaaacttaatggcgcgacttaaaattggagtaatattctatttagttcggactacatcgtcgaatgataagtacggtatcaagtttatcgaaggtgatgttttagaaaccatatgtattgggtgccacaagttattccgatttgtttcagtttttttcctttcttatttttcctgcattcctagttagtgcagatcgaggacatataagtattttttttgcgaggatatatgtattttttaacttaataaaatctgcattatctcgttctattattcatcgtctgatcacatgcatgctagtttctgataagaatagtctttcttcaatatttgagggaggagaggggattgcatatatgcaagtgcagaatttctacaacaggatgagttgtaaagcgcataataattcaccgaaatataagaatgcacacatgcaagatagatcgagaattggatagatccaagaatgctcaggttggtatgtattaagtccattgttgtggtcgtcccagagcttagctattgtccttttaacaaattcttatccccttagctttatcatcaacttgcatgctctgcgtgtgcaagaggattccagaaaagaagaaaaaatgtgctagagtggttgtattaaggtagcagatttggtagtcctagctttgcttgatccattgctttaggccatgaaggcatataacggcagggataagcccagattagcaccacatgacatggaagtgaaaggcgtgtgctcaccaaccatgtaaccactcaaaatctcacccttttcttttgtccacaaagctttttctatatgcaactatagtcttaattaattaattcaacaggaggcatcttattaccatttgaaagctagatatatgaccaggaggcatgcatgtacacatatatagctagctagctatttgatttgaacaatataaaggccttgataagtgctactaatccataacaacactagtagtatccgggttctcaatcacattgtggaccattttggcatatcattttgtgcgctcaatcttgataagtactccctccgtcccataatataagagattttaagtttttgtttgcaacgtttgaccactcgtcttattcattttttttaaaattattatttattttatttgtgatttactgtattatctacagtactttaagcacaacttttcgttttttatattaaaaaaaattaaataagacgagtggtcaaacattgcaatcaaaaactcaaaattctgggacagagggagtagttatttgtgcctttaatctgacatgctagaagggggtagggggagtgtattaattatatagtgtaactcattatctgattgcactaagaacgatctagctttgcttggcttgatagctgaactgctgaagtgcatttgccatggcagccatgcaaatccatactgttatactagtttttattatctcatactttccaaaagaaaaaaagacgttctactctccagcttttgtttttttagaaaaaaatgcaggcatttgtcgttctttggccccacttgtcccaggttaatgtttgggatttaaagttttaacctcatttccactaggacattacgtgttgctttgtatccatcgttagcagagtagagagtgtgaatcaagcgcacatgggttaatccagaatttcagatccatcatccttgtgcaaaggaaaggatatatgatttcaacatacacaagagctagtgtgaccagtatagcagagtgtgcacccacatgggcatatatattaggaaaaagtacgaattaccatcttccccgaactatcgcggtcgatcgaattaccccactgaactcgaaaaccagatatcgctcaccataaactttcaataccggacaaaagcaccccctaactcaacatcgcagtggttttggtcctaagtggcagtccagtcagcaatttttattttaaaaatggtggggcccacctgtcatagtctctcctctctatcccctctcacctctctatctctctcgtctatcgctcgctcaccgctgagaatgagtggagcacgcgccggcggtggttggcccgacgcgatgggacggctacagaagccggcgaccgtggaggcgaaggcagcgcggtcgaggactagcggttgcgagtctcctcaaggcggaggccgaggatgaggcgtgccggcggtggctagcccatcaggctcaccaggcctcgtcctcctgtggttaggggagtagcggctgacaccggacttacgcaagaaggcagccatggcggggaagaggggcgaagcggcggggcggctgcggaggccggtgaccacggaggcaagggaggcgcggtcggcagcaaggccgaggcagctcggggtcgcgggtgagcgccgtcggcgggtgaggcagctcgaggtcgcgggtgagcagcaaagagaagaggagacgagtggttcaccgtcgccattgaggtcactgttcggcactgaggcggaagcggtggcgctgctaatgctcccttccctggagacggcggtgaggctcccccctctctctctctctcctccttctccactcaggcaggcggtgagcgacaaatcctcctcggggaacgcatggcgcgcgcggtgggcagggcgaggtgggcggcaccgaggacattggaggcggcaggctatcgtgacgagtttgccgcccatccccgtcgtccccgtgcccgattgttcctctgccgcctccactccaatctcctctccttcccgatgtcctcgccggcggctctaccccactggtcccgtcacgacgtgccctccgcaatcttgccgtgcaccagagggaagaaagagagaagagagattttagcagtactccgactgcagccgcatcctctgcgtgctcgctgcgccgcccttgtctccacctccatgccgctgccggcacgtgccgccctcgcttccacctccttgccgctgccggtagtgccgctctactgtgtcgctccgtctccccctgccgccgtgcgctttagaaggaggaagaagagcgccgcgtcgtgccgtctccccaaccaccacccgctttagaaggggatggagaagagagaggtggtggggattctaacgagtgggaactagcgatttttttttaaaaaaactgatgattggactgtcacgtgtgctatttgaatcctaaaccgcttgcaacagtgagcgatatctggttttcaagtttagttggtaattcggtcgatcgcgatagattaggggggtaattcgtactttcctttatattatggattgagaacaagaggatagcttattcttcaacgcatccttgtgacaatctcctgcactcccagataaacagatcatgcatgagcatatatatcgatcttgttagaagatatagctagctttgattgtcagtcaaagatatggctccctttttatcaccatcacagtcaggggcaacatggctagaggtcacttcactatcaacggtcatggatcgagtatactgatccacttttaaatgccgtatccttacatactccctccgctccaaaagtctgtctaaatttgtagtactaaatgtgttatatccatccaaaaatctttatatatttggatggatatactacatacattttgtcctttaaaacataataactaataataactagaaaggtggcccgcgcaacgcgcgggcatccatattatttaaatataagattatattttgttcgtaagatatatcttataaattaagggcaactcaaggtcacacgtatctaaaaaattattcatcgaaattttaatagagaatggattaaggtcacatgacatttgaatcaactgaataataaaatgctgctagataattatcatctctatcatttatacacatagtgtgacagtgtgaaatatgtctataagctaaacttgtgagagtaaggaaatcaaagttctttggattgaatcgtctcattaaggcacatgatatagtgacaaccaaattaaggatagatcaaatgtttgatttttttttcctatgtagaagttcaacaaattcattttttttcacagttttgaagaaatttaatggtatgttaggacacatattactcacacaattcattggcttggtaatgcaaaatattagcaattgtttcaagataatttctttcttttttcatgcttgattgttttctttgaaacaacatttgtttgaaataagatcaaaattgtataaatttgattagtatgtgtagtgtacttatacatttgaagtgttctatagtaaagtttgcttttgtcatataatccaattgtatcaaataatattgttttatcatgtaaattataatcaataagttcaatatgattcatatcatcataattaatgaaacttagttaaaaaatatgactacctaaaaattattgttcgcttatttgtttttttctccttacttaatggtgcctttgtcattactctgaatcaaagaaaaagtggcacatcataccatggctcatatatagctataagctattactctatgtccactcattgctcagtagtgtccttcactcaccgatcatcttgtgatagtcatatagacaatatttatcaatttgtttagtttacataaagggcaacaccacattgctcctttctcatgcatggtcatagagtccaaaatgatttttccaaataatgctctgcattaacacatgtatctagaacattaactacataacatatacgtgtggaacacatggtagttttcgttctagattcacccttatttacatacatgtaaagagtgtaatgacaatcaatagtgacacctattaacaattgttatatacctaattagaatatgtattaattatattcatctcatgactctgactatctttgatcaccttgagtagtagaaaaaaataattgtactacgggtttaggatgggtgaaactaatggattatttgatttttatgatatctatttgagggtataaatgaatgaattaacttatacaaatttgaaatgttgttctggaaaaagcgctttaagaaaaaaaatagaattgttttcccgaaacacatcttttaggaacatgggaaacaatagcaaaatgaaaatacaaaatctagatcgaaaaggaaaaacagggagtaagactcccttatattatataagtacaattaggtaatcaacccttcgttttagcttacatagagttgcatgttgcaaaaacttaacccaatggacttcattattttccttctgcaatagtcgttcaatttgttttcactttattaacaaaacaatcattatccattgatgatttgatgtgtcctcaccttggcaaactggtacggtgtggtgatcatgatcaccttacttgcctcaatctggtgcaccgcattgtcgtggcgcacgcatgcatgtgttgtgtagaggaacggactgatktttttggaggatttttttagaaaaatagatctgatggtttattatctatcaatttaaatgaaaattgatagtcaaatattttagcagcaatatagtctcaaatattgggcccacttgttggtttaaatataaattagtcgatatagattataaattgttaatttaataggtataaaatataatggtgtaaaattcaaacaattttatatttgaaaaaaaaaattaatccaccagttcaatttaaaaaattatcttatattattaatttaataggttaaaaatacaattttttaaactttacttttaaacattatagagctaatttttagttatatttatttgtaaagagttaatttatgatgatatatatagtctatgtttgtaaaattataattattttaaatttattatcactaatataactgttttatcacatgtacatggagaaagttggaaaggagagagaagcagatgtatttttttaattgatgtatataatttgtgtttttatgttttttttgctttaatcttcttttattgatagtgcgttacttacgtactttgtattttgggggtttttttttttacgatcagtactttcgtgaacgggagattataagatggaagtttttttaaatagatctaatctaacaataaataacagatcaggtgttttgcttgtgttttgcttttcttttagaatttataggaatttctttaatttattaagtgccacatgacagcttgagatcatttgtataaaatttaataaactttttatatataatagataatagattgataaatacataattgaaatatagaatgataaagagtgttgattggatatcttatatacgatctagtttaaccgcatatatttatagagcgacatattttatttttaaatagtagaaaatcctactttaaaggccatcaatttttttaatgacactacgtacgtatgttttttctttactttatcgcttttatccggtgaagagtagataattaagcttcatcgtttttatttcagatgtatgtacgtactgaatactttcctctttcttttccgtcccacaggaggaaactattactttttaaaataatacatctaatcgaatggtccataatatcgggtccaccaattttaatgaaaatcgatggtaagattaaatagtgccacatgacgacctaagagtgtttgtaggagtgccacatggcggcttaacagtgtttgtaggaagtttaatggacttttagtatacaatagatagatagatagatttctgactataaatttagacatgggttattgaggttcatagcatagwtttttttttaagatggcttgagtatatgcaacgagaaacaaaggcatgcatacatgtcatcatttggttaattagactattgcaacttgcaatagctacctacctgatgtacgtcgaagcaagtacaagttgcatgcaagctttattgtctgtgtctgtctttaagaaaattaaagcagggagactctaggataagaaagaagaataagctgttcaaaatagaccaactgttcatgatgcttttcaccttcctttcgttaattagaagcatataatcccagagatttcagcgtcaaggggaaaccatttgtagcttggtagcacaggttatataactaaagccaccaaccaatcatttgggctaagatcgagcgatactgttacggtgctgttgcatttacttttgactaattaatctgcgagctaccaggccaaatggatcagccccaatgattaacaacgctcatggattggaactaattacacagattcatcccgatctgcaggtaacaaaaatatgcgaacctttctgcagtcaatctatgcaatggtacagaagtgtaaacagataattggtggcttgcagagaagatgaatctgaattatataagcaggttgggtgcttgttgttggtggccatgtgagtatcagtatgtgacccaggatgactcggatgacacagctctgctcgccgaccgatatgaaaatgacggtgatcgactgcgactgccaccagttggcggcggccggacgagaccccccgcgtcgttcctcgtcgtcctccaccttcggctccctgcttaaactgcttcgccgacctgttagactatttggcaatgtacctgtgattggctcccttccctaaacctttcgatcctgtttctataaatcggttgagcgttctatagaagattgatgcctatttgtacagctaccatgtatcaacatgcatgattatcgcattgagctgtatcatccaacattccctgctccggcggacggccctgctcgccaagccctggctctgttggtctctgcctctgtctcaaacagatgttgttcgagccattgactggatggctaaaggagaacagagagaaggaaggatgataggacgacaggtgggccctgtaaattcattattattatttttctgattgggatgttatgttggcaccacgtgtaccagtttaagtcaagacaatccataagggataatttatccggtacgaaaagcttagggtgtaaaatatttggttttgtattttccgggggtaaatcggattggcacaatagttcagcagtgtaattcaaacttttttttaagttgttctactattttttactctctatcccaatataactgcaacatagtaaacataggctctatttagttcacaccaaacttccccaaactcccaacttttcatcacatcacatccaaaacttttctacacacataaactccaaacttttttttttccaaactaccaactttccccaatttcaggaactaaacacagtcatagtatagatgtgacatttgtactaggttgcatttatattgggacggaatgagttttaaagatagtcaattcgaatatgctaccacttggtgacaaatgatgcatataaaaccatcgtatgtacctctatttaggggggtttaagattctgagaattaaccaatgatagtcacttagtaatcaatttagcgaagctggaaaatgcctttagtttatttttattccacagttgctgattcttcaaatctaaattgtttggagtagctgtagagatttactaatacacattgtgctgcttgaatctttttcaaacatgaccatagaatagatgataaataatcaagatctctctttgccggagcaatggagggaccgggagaacacataggtgagagtgagtcgatcaaacgaaggacatggggtctgtttacttcttgccctgaaaatcttgcctgactcagacgcaattctccgaaatttaattttgtgtgcctgcagatcgacctgactgagcgagagccctcagtcaaccaaacaaggccatgatcccccgccgctcgtgggcgcacgcacaggccatgtgacccatgttagtaggccgccgcgcatcgatctggacgcgcgactgcacctacgacgttcgcctgaatgcaagcgtgatgcatgcacccgtagacgttgctattggagatatatcctatatgcagtgtagtacaagtactatggtggtagtacgtactatattttgctgcttttgataacagaggattatgagaaaaaaaaataccagtaagaaaaggaaaagaacatacgtgttcccagtagattttggttgccaacagtgcgatgacatagtaattaccggcgaattaaccatgacaagctgacgcccaatggatgaagtggccgagctagcactgcagattaaacaacaaattaacctgcaatggccaactaatttagctagatagccaagtttccgtgtcagccatgtgcgcccgaggagggctaccttgtgcagctgccggaggaattcgaatccaatgcagacatgcgcacattcatgcatgcagctgcaggagcatgcaacggcggctggtcttaattaatttgctgaattgagctgatctgcatgcatgaacgcgtgaaggcacaggcagagggccggccgttggtcgcaatgcagaggcagctaggcgcgcgtacagtgatctgcctcgataccgcgtcgatccattcatcggctggtccgtcgtccgtccgtccctgtcgcccacgccggagcctagagatctgtcggcctttgggcagccacccaacagtcaagcgtcgtgcgtggtggctggaccattttctcgtagtaccaaactgcgtaccaaaccgatcgatcattaactaccttcaagagtactctctttactgtactactcataatcggaggccgtatgtcgactaaatgctgaggtggagaaaataagaaaggagagagaggtgaagcaggctgtaaacttatagccggtttggatgtaagaaccaaaatattctctgagagagacaagtgcgccatatattaattctaagagtaaattgcgttgacggtacaagaacttggcgggtgggtgcgattaagtacaagaacttgaaagttggacatatcgatgcaacaacttgactaggtggatgcgcttgtggttcaaaaggctgccacataaggttttttgccacgtcagctttccaccttggacaaagtctacgtgagggctctttacacatcataataattttgcacaaatcccctgctgtttttcccatttgaggacattaattaatcaccatgagggataggacctcaaatatgtagcttccggctgtagcggtcggtgggactcaaagccggcgtgcggcagcagtcgtcgggactcaaagctggtggcggtggtcctcgggacttgaaaccggcgtgggtcagcggtcgatgggacacgaagcccggccacggcagccgtcaggactcaaagccagtgcagggggttgttagatctcgaagccaccgtcgctgcccatggccgtagtaggttcccttcaactgatgagaatctgaaccwtccwtytttttgtgctgtaattctatgggattcttcgtccatagtggaatcagcatcatgataactgcatctggtgtaccacagtgagaggactctttgactctttgttgctgtcaaagcagtatgtgagtgaataatggcaatgtaggtgcttttgtaaatactctggaagtgcttttgttatataagggatacatgaattttttagctacttaaatgaatggtgaacacgatgtagcaaatactgcttgtattctctcttttggaattgtatgtccaagtgataacctgaattttgctaacattgctttgaccagtagcaatttctgtacagacatatgtggtatgttttcttgtccatacacaaatcctggcagaaatgtgctcctaatattctaaaatgttttaccataaatagctgttgtataaacaggaaaaacaagagatgttcctgaattggaacttagagcaatgtaacagagcacagtccaaaatgaaacatgtctaattggttggcaaaaaaaaataaaacagaacttctggctaaattataagctaatcctttttatcaagttgaatatattgttcactttttctttgtctcctcccttttctttaactctgatttcttactgggttgcgtatttctcttccttggctcagcttttctcttccgaattgttgccctgccctctttggttgctgtcgttattggcggctgcactgcagcctttcggtttgtggcaataaaatttgattccggcagtggaccaaggtcaactctagaagagttgtttataatgttctgcagaaagtacattgaaattgtgaaaactgttttataaagctataaaatggaaaattggcaaagcattgtctaacctctctcgtcaaggaagacaatatggtattttgtaactgatcatagctaggttggttggttaaaatgctagtctgactgttcagaacttcaggcatgacctgcagaggcataaaatcttcattaccatgttgttctgaaggagcagcactggcttgctgctttcctgcttttatatcttccttcctcttcttgcagcccttcttgttgtgattaactccttgacagtagctacaatgcatttgcacaccatgcttggatatttttgttcctccttcaagttcatgtggctgctttcttctacatctcctaggccttccaactttcttctcataaatcggtgggttcacctttggtccattcatcttcacccaagatgccttgtccctaacaggcattatgttaaatccataggcctccatgtatttttcagttgaataatagaaggataccacatcttcaggttttgtcctctcactcctcaagcaagaaattgcatggttgcatggtatccctgtcaattgccatctcctacattcatagtgttttgccatgaggtcaacaatatattgatgttctttatcagttacttggaacacccctttaccagcaggtaatatatagcatgtatttgacaattctacatttttatcaacctttttctgtatttttatcaacctttttctgtatttttgggcatatgtttcccgaccacttctttacacactcctcttgtttggtatgtatcctattcatgatttggctcctaatcttttcaagcatggataaaattggaagctctctggcttcaagaatgtacttgttgaatacctcggaattgttgttgagtagaatatcacacttggggaaatcactaaagaatgctctgcaccactggttgggtggtatttcctccaggtaggcataagcatctttgctaagagacttcatcttttccatgttaaattcccactctggtatagtacttgaccttgcaattgcccacaactgattctttagagtctcccctttgtatagaacatgaaaattttggtacaaatgcctaacgcaaaatctttgttctgaatctgagaatttatccctcacagctggtaccaatccctacaaaatttaaaaaggaagggtgagtaccaatcmattgaacagtaaataatttcaaatatwttwaaaatgacaagttgaacaaaatcaaatatgatggaataaagtcataccttctgcctgtctgtcatgatagtataaggtccagtgttgtcgataaggagatcttctttcaaagtattcaaaaaccaaccccaactagctctactctccacctccacaacagccatggctataggatagatgcagtcattaggattaatgcctactgcagtcaacaaatgtcccccaaacttggttttgatgtggcatccatcaaggaatatgataggtctacaaccacttaggaatcctcttttgcaagcatcaaatgacacatagcatgtatggaagcacatattttgtaggttcaagaaaaatttactacctggatttgaagtcctaagctcttgtccataatcccacaacatatcgtattgtgctatttcatcaccatatatcgctttcatagctagtttccttgctcgacctaatttatgtcttgatggtgttacatttagttccttttgaactaccttagagaaactcttgagtgtcatcctatcattatccctaaagccttcaacatatctatttgcaagatacctggcagttacataattcaactcccactccttctgacatttgtggccatctatatatctttttaccataagacaattcgtcctgctgtcctcagctacatccaacatccatgagcactcttctttacatttagcctcaatwtttttcttggtatttcttgtaaatttaattgcgactctattcttcacactgtattgttttattgcttgcctaacttcaatagcatcagagaacaacatcccaaccttaaagattggtgactccatgtccacagcagccacaaagttcttaaatttcaacttaatgccttcattatcttcatcagatgactctggaagcaacaagccttcatcatcagagtcatgttcaggaagcttaacaaccatcttgctgctagccacttcttctatctcaacacagttgtcaaaaacatcatcatcccccttgtcagcctcatactcactgtcaacaaagtcaggatctacatcgatgtcatcgtcatcgtcaatgtcatgaccttctataggtttaacatctgaatcttttcgattcattgtagacctctcacggctagccacgataggtatagaagcaggggtagaaagagcaacatcatcccaattgatggattcaagaatgtcttggtgatccacatacaaaagcaatgtcctaaaccttggtgctaatacaaccatgctgttggtgcttgcatcagagttaattctccttagcccatcactgaatgtcttcccaggcaataaccaataaaccttggccttctcagacatatcatatcctagttgctcaataaaatcatcaagccataatgaagaccatgtgtcactgtcacaataatcaaaccaatccaccttttcatccaaataaattctgctactaccaactccagagaaaaaaccaccatgatgtagttcagttgtgaactgttcatcctcgcagcctacatcataagacaaaccattctgatgtgtcaaatcgtaaataattatctgatctattatacacaaaaagattatacagagtacatattcatgcttagagcaaaaattatttctaatcacaaactattcgtgaaggattacttatatgacacctagccttataaaacaccaaagcagtaggcggaggcagcgccgggccaccacaggcaattgcccaggctccatgcatacaacaacactgccccaatgcatataacaacatattactcattaatcatatacttaatcactttgtctcataggttataatggagattaattagatgaataaacagaaacgaggaagcgaaggaccaaaccatcaaactgtaagctacagaagaccaaactatacaatcacaagattcaaagaaaaaacagagaaacatgcaattaccggtacaaatcaatgaaaacctaactttatcaacaacaatcaattgaaccaaaaaacgaaaatatgtaatcactagggagttatttaccataagaaggagggaacgccccaagaattcgccgagtcggcgccatcccagtgcacgaggcgatcacgtcagatcagttcgtaggtctgaattactaagcgcaccagtttgggatgattctcagatcagcgtggctcatatatgatgcccgcgcaaccggtgcagaggcgatgcagttttgctgtgcactcgctgctagccgccatcgcctcttgatctgttgaggtgagtaaatcacggatagaaaaacagaaccaaggcacacatattttttgctatgttaaaacaaaagaaccaattagaatataaaaattactaggaggttttttgtaaaatragatgggataggtcactctatgatgctgatgctaacgtgggactttgtccaaggtggaaagctgacgatggcaaaaaatcttatgtggcagccttttcgaaccacaaatgcacccatctagccaagttgttgcattggtatgtccaattttcaagttctcgcacttaatcgcacccacccgccaagttcttgtaccgtcaatacaatttactctaattgtaaatagctaactattatatgtgtgtgggctaagagaatgcggcaaagaatcttatagccaacaagtcagctgtattattagtcttgctctaagtagtagtaggagatactccctctcagtactcataaagaaagtcgtataggataatgtttaaatcaatcattgggaatataaattatgaataactctttggttgtttagtttgaaaatgtaaaaattatatgaatagatttgtcttaaaaaatactttcataaaagtatacatatatcacttttcaataaatatttttatagaaacaagaagttaaagttgtgttttggagaccgtgtcgctgtcgaaaacgacttcctttacgagtacggagggagtattaaattccgacaaataattctctaaattattacgagaatatccgaccatcgtaatagtttagagggattaatcgtcagactttaatgtcttctactactcatcattcatgagctcataaaaactaaaatttttgaatattccataaccttctacttttcaaaccggacaataagaaaaacccatataaaactagtcgggggtagatttggtctaagctgacttacttgatacaaacgtgacagatcaattagcaaaaagcattaaaaataattcatagaacacatgtcatatgcatcacctctccctctctttatctccttctgttagacggaggcaactggaggcttataattaggtcgaggacaacgtctgttggcacaggtggaagcagttgaaagctcaggctgtggagctcgagctcttctctcggggatttgcatctggtttaacagatactttcacctcgtgccaagtggagcaaaataatggtcacacacaacaccatatcataaaaggaaaaaagatatcacaaacttctttttctcaccatatcataaaaggaaaaaagatatttgtaaaacatgtgttctacaaaattaaactattgcttcacccagtcatatgcaatattatttgagaacttattttaatctcttagagagatgtactcttatttttttacatgtcatctagataattaaataataataataaaaatataagttagattaatatgtgatatatcattctaattgttttttttaattttaacttgtacgagtgataatttgcatgcttgtaaaatgatatatcgcatattatatattttattattttttaactatttggatgatattttttttagaaaatggattaaaatccggtatctacatccacaatgaacttacatagccggccactatttggatgacatatatatgtatataagaaatgatgagatagtttagcatctattttcgtattatttcctttgcagcggacatagtacatgacccacagttaatttttggaacgagtaaaaaaataaaagaaatgatcgagatgacccacagttaatttactcctgactgaatcaccggcgggcaattcagctatgcaagtttgtcgtgcatagcagtcactactgttagtcctgcataatcagacaaagacgcatacgcattgtcgtggagtgagctagagccaatgtgcggcagagtgtgttaccacgactgactgtcactgcatgcagcactaataatcgcttagcatgtgatgcacgtacacattattgtcagtcaatgtcatgtcatctctgcacggaccaatattatctgcgccaatcaggaatataatcaactcagctagtactaattgttgattactttgatcttcccttttcttggctgtattcttctcctctctttttttctttagcttagctagaccagtaggagtacgttgtcgtagtaatagacgcatccaattggcttgatttgtctcgagcatctggtttccttttctaataaaaatcattagcacataattaattaagttttaattattgtaattttagaaaactgatttatttgatatttaaaacaacttatatatagaaaattttctcacaaaacgtattgtttatcaattttaaaaaatatggaaataaaaatcaagataaaacctatttcatctgtccctaaatataagagattttgattgaatgtgacatattcaatctggataacttgtccaaattcgttgtaatagtatgagttacatctaatcaaaatcttatatatttagagacggagggagtatataataacaaaatagcacgacctagatccgaacaaaatctctccaggctccatagtatttatctgagcaacttgtgtgggcggcgagaggcagctttacgctcactcaccggccgctgatgctgcccatgtttcatcaatacgacgaaatacaatgtatactactacctacatctacttttgatagtcatattttatcttggcacacagaccaaggataaataattctacttgtcatctatttaaacatgctactagtcattcctcgtaaacaaacaattcattaatatttatatttctcaatgcctatgtagccaatcatgtgtggaagaatggaaagtcacgcattaaatccgaaaaaattattaaaatgataggttgttggattgaaatatgactattaaaaataaatttttcagatttagaaatatgactatcaaaaataaatggagggagtactccccaaaccagctacacacaaagggagtggtcaataattacctggtccatcgatcggttccgacgattagtcagaaaaagaaaaataatcaagagaaggccaatgagcatcgcgatttgtgttcgttcagatcgccaatcgatcggtaccatgattgtgattccaacgacggaattgttctcttcctagcctcagggccctataggcaattgattaaccgtcacctgcatggatacactgtacagtacctgagtaccatacggggttcaccaagaattctgtcaagttgaacagtattgtaagtgcaagtagaataaaaactataagctagctaactacaaacacctgtggtggaggaagaaaaaatggcttcatatttgttgtcagttatagtacaacctttaaaatagtgtataaaaggtgaactaagaattaatagtactgtagtactccctctatagggcaaacaaacccgataactttttactaataatcatactaactatatctatactaattattatgtacattatatcgctagattcatattttcaaatactgatgttaatttcatatttcttgggatcatagaataaaaaaatactagtcaaaatatattattgaaaaccgtgtaaaaaatataggttttataatttgaaacaaataaagtatatatttatatgaaactactgtatgtgtaggctattatattagttataaataacatggttaatttttagagctatcagctgagtatactattaaacgttgctcttatgtcgttagctgtgatatgctgcctcgggcaacctagaaaaactcgaggagtcgcgcggccacttgcggttgcgggggaaaaaaacacgaaagcataggcatccacgcgatcctacgatgccagcgcgtgcggatgccgcgcccaaaaaccggtagctggtttcgccgccccgtagggagcgcaggcagtttcacgcgcgcggccctgtttcgctccgcgcaacgcgcgcgcgagcccaaaaaaaaacgacgggggaatcatcgcctccgctgctgggttgtggcttttttgccttttggcgcctagctacgtaccaacccacatcacgcgcgtgcgcgtggtggcacactggcatgcatatggttgcggggggaaatcggggaaaggaaaacgcgcggccctcgtagactcgactctctacactcgcgcgaggcgagcgatgttggaggggaggggggttgcgtgtgtgacaccgctcgggttgtccctctctcgctcgcggatgattccctcgcctccaccatcgatcaccaccaccagcagcaccaacccaaccacagcctacgcatacatgcagagcatatatgctgctttcctttccatagcgaacacccagggtttttcgggctagcttcacaagatgatagtctagatgaactggattcaaaacctcacccccttttaattatttgatattagattattttctaatattcgtgtatttttagcctatagccattatatattatgatgacgataataatccgacgacgtccaaggatgaggcaggttaattgggatttgtcctcgcaaaaaaaatgaaaaaggtttattgggatttgtgaggaggatacgactacgaggacgatgtttgcatgttgacctttgttgtggggcaggtcacaccgggcaacaatcatatgtgccgcaccatgctagagtgctttattttgtttttgttggttattcgtgtgagttatttaatttgctccacgctttttaaatatttaatttatctctataatcaagaagataatccattaatatgcttaataatcatgtgtatgcggcattgttgtttttgctagtatttcataaaaaatattacaaaagccaaaacataaatcaaatatgaaaatttaggaaaagcaaaaccataagattacaaaatagataaattaattacacatgataagctagtatgcgttgatgctgagattctagaggattctttttcaatttatttcttcaaaactaaaamcatttwtatgctctctctctcccyyctttattaaattgtatttgacatgtagatctcacggagtaggttgaagcccatttggactaacccatgtcaagttcagggactcgaaaaattgtttttwwgagtgtagagattagatgtcatacccacacaagtttagggactgcccgtgcacttcactttttttatgtgtttgtgaatgtaaacacgaaaggaacacaaatttagattaatgcatcgcatctcatttgtactacactaaatgaatatcaaacaaaaatttccaaggtactgcacccgaagagatagggaatagaaagaacacaatttttkgrgragatagattagaggaagacctttagttgagaaatatccaacactgtattacttttaccaaaaacattactagctgttgcaaaggaatttaaggagggtgggggaggggctatcaaatttagaaagaatatgcgaagtgagatagctcatctgtttagtttttctagaacctatgtacccggatttgagttctaaacttggtctttttataattctatggagagtaataatctgcaattccctttctcagagatttgaatgttgaaattacctttcaaaattaaggtggtctcatatatggttgctccttagaaatggtatccttaccaaagataatttccttagatggggctggaaaggggataattttcttattggtataataatctaattgtgaaactattggacacttatttatgaaatgtgctcaggcttgatttgtggacaacgtaattaatatcactttcaatttgggtgcaatttagaatatcccacacatatttgagggatgaaacaattttggaaaagaaatgaggaagctaatagaggtaggtgtataaccgttgttccgtgagcaatttggacaaaaagagaaatggtgcttttttaaaaaaaaatttactgctaatagaggtaggcttgattactcgcccgttggagcgaatcagacatgtctatttttgagtttcatcccaaactattttaaataaatcacaggaagagacataagaaatataattttatgagtgagggatgtgggtaggtcagacgctaaataggatggcaagtgacacatataatacgaggtatggccaacgaccttggagaagaagactaagatgaattctctaagtcttggcatgaaggttttatttaagaagaaatgagtgaaagaagtccagggttttggaaaaatgctacttatgtcatctaattttggaccattttctatttttcggttgttaaatacaatacttatgttaagtgttttttctctctgtagagcaacttgaaagacaatgggatggcattattaagtcaagtgtcgttgataatgggattatgtactatttacccatttatgtaaatagaaatgatagaggccttgacctctctttttggaagaagataacacgacttgagaagcatactaacatagactatcttcatgatagtgtgagtgtgatgtgacgtctcaatgccatttgaaatctgtaggaagccaacgacatagtctatttgtaccaccataaacattatctgcatacatattcatactagtaccaagtctaaaatgtcaaatgaaaaagttggagagaaagataaacatatattggggttgagagagggacaatacaataatgggtggagaaagtggataagtaaaggaacaccaatatttgtgtggacaatgaggctatcatgttacgtatttttatctcgacagagggaaactattttaatcccttgaggggatgtctcctcattgtttgcatgtcacttaaatggttatgaaaaaattgaaaaaatttgagaagatgtattaacatgtgatatatcacttcacaaacatgcaagttcaaattcaacttctacatctcacaatgaaaaataaataaatttgactgtgaatatacgttaactagttgtagtttaatttgttttttcgttgcgagatgtagaagttgaatttgaatttgcatgtttgtggagtgatatatcacatgttaatacatcttctcaatttttttttaaattttttcataaccatttgagtgccatgcaaataacgaggggacattctttcgagggatcaaaatccactcccctcgacagataaataaccaattttttttgcgaagaacaataacttttatgacattattaatacagatgagttttatatttataccaaactccaaataaaaatatttagcagtatttgctactcgtgttaatctctacagtacaagttctcaagaaaattacactttgccccgtggggagttctaatcttccagtatttctcgtcgctcccgatgcaattgaaaaaatacacacgagttgtaacgaacaaaaaggaaaaaccgcacgggtttatcatcaaagctggatctgcacaacaaacagcctcgcaacagaaagagaatcaccacgggacaaaggcggtgacgtgtcacgcggcgcggccccacacgtccgcgtgggcccggcacgtgccccgcggcgcgggggccacctggcgccgagctgggctcgcgcgggtgtggcgacgtcggacgcgtccaagacatcggcgtggcccgtggccaccaccaccaccggcgggccccgccgtcgccgccgccgccgtctccctcctccctctccaactataaatacctcactcctcctcttcccgcctccacacaagcaaaccaatcatcacctcttcttcttggggtgttcttgactggaaggtttctttggtgatggcgaaggcgagcgtggtggtgcctgagcaggtgggcgcggcggcggcggcgcaggtggggtgcccctgtccgggcacgacgctgttcccgtacccgccgccgcgcgccgggatcgccgtgcggcgcaagtgcctgcaggcggcgcagcagctggagctcggcgccgggctgcgcggcggctgggtggagtccatgcgggcgtcgtcgcccacccacgccaaggccgccgccgccctcgccgccggcgtcgacgaggagcacgccgcctggatggtccgtttccgttcaccgattgatcgatgttcgtcgcgttcttggcgcgcgcgcgctgacactgacatgaaccgtgcatttccgttcgtctttgtgcaggcgaggcacccgtcggcgctgggcgagttcgagaaggtggtggcggcgtcgaaggggaagcagatcgtcatgttcctcgactacgacggcaccctctcccccatcgtcgacgaccccgacgccgccttcatgagcgagacggtgagcttgagctcccctcccctgtcacctactctgctcctccactcatcatcatctcacacctctctccttcctcatcagatgcggatggccgtgcgcagcgtggcgaagcacttcccgacggcgatcgtgagcgggcggtgccgcgacaaggtgttcgagttcgtgaagctcgccgagctgtactacgcggggagccacggcatggacatcaagggccccgcctcccgccacgccgccgccaagtctcctccccacaacaagggagtcctcttccagccggccagcgagttcctccccatgatcgagcaggtgcaccagcgactcgagcaggccaccagctccatcccgggcgccaaggtcgagaacaacaagttctgcgtctccgtccacttccggtgcgtcgacgagaaggtaactgatcgatctgcaagctcgattggttgattggcttttttcatttggtgatcaattgatgatggaattgggggtgcagagttggggggcgttggcggagacggtgaggagggtggtgagggagttcccgcggctgcggctgagccaggggaggatggtgttcgaggtgcggccgaccatcaagtgggacaagggcaaggccctcgagttcctcctcgactcgctcggtacgtgcagtgtgttacaactttgccattcctcgtcggcaaaaaaacccattggctctgctccgccgcaggtttcgccgactgcagagacgtgctgccggtctacatcggcgacgaccgcacggacgaggacgcgttcaaggtaaataaatactaaccgacaaaaattactgcatgctgccacgctacgactacgtgtagcagcagcagtaacacgagacgctaccactacttcgtttgcaagtggttcgacgaacgtacggccggttcgtcgcgtgcagctagcgacaacgtacgtcttcttctacgttggactaaccggcgagccgtgtgcatgatccgggcaggttttgcggcggcgtgggcagggcgtggggatcctggtgtccaagcaccccaaggagacgagcgcctccttctccctccaggagcccgccgaggtaattaagcaaaaccactcgtacgcacgcatgaaaacgttcgtatcactctgctcatgtttgtgttgcaattgcgacaacaggtgatggagttcttgctgcggctcgtggagtggaatcgcctgtccaggacacggttgaggctgtaacaattgatgatcatctggcatcagctaatttaaccggcgaggctagctagagagaagcgcgtgatctgggccgtccgagcgattacatcggcagggtaacccgtgacgctgatcgatcgtggattctacaccaacacaggtgctcgaaaatggtgtccacattgcagaagcgcagagagctaattaatcaacgacggacgagagagactgatggctgtctggccattgttgtgccataatcctgtttagttcttcacctttcacctttctcccttcttcttttttcccatttggggccccccttttggtaccaaccatgtaaattccgtactactagtaccttgtcatgcacaagaggaagatcaatgcaaataatgaagagcaactaatgcaagtatatactcatcagcacattttctttctgctagcttagctatagctcttgctctggtctgatgcaccaatgatgcgttacacatttgctttagctgaagaaaagctcagtgcactacccactgccggcacaacgtagcaacaggctgaaccgcatattggtcaattgggtgagtgtaaagcttgatctgctcgttgatggatgttgttaggacgagcagtaaactgggcctcactcacatgatcggtagcacggagtggtaccacggcttctctactgctctctgtcaatggatccaacgacacattgccttgtcatgttcttcctgtgaatcatgtcctcatctctgaagaagggcctgccagagcaacctctttcagcgtgttcttggtgggcgaagctttcactgatgcacaaaatgagcagcaatctttgatgaggttctgtgtgctcgttgaccactaatttgttcggatcgaaagctcgacaatgtcagtaactatcactgcatcatgctctcgcacacatcgagtgaatgagacatcctcgatcgactcatttaagtagattgcagtaataactgaaacgagcatcgattaaccaacttggatcacttgcaaacaagggaaggtaggtacgacgctgcatgcagattgattgatctggtcagatcataatagtctcgcttaaaaattgcacatgaatcgcactcattttggatttggatgggactgatctcgattgtacacgttgatctgccttgaagagttacacgacaaacacagccggcccacaggcaaaataatccttggaaataatgcagagaaaagattagcatctgcaagaacctcgtactgagaagctagctgttggatcgatccatggtgcatttttctgcatccacagtggggtgaaggacttaaggacaaagtgagcaatcttcaacagatatactagtacgagttaaccataattatgtgttagatctagtgctagctatgatctagtagttaaaatcatttcttcctcctaacaatactagcatatgcaccgataataatcaacgagagaagtgctagtgctggttctacatgcctacaatccacgcatcatatcaatcattaaggttgatatttcagtccaatggcgcactatgagagagcccaaaaagtaagaaaaatgaggggttaagtgattttactacacacacaaaacgactgtccacagaaaccatgccgatgtcactgataggtgggccccacgggctggttagacttcggggacccttttgtcggcggctatataagaaatcacagtacacatacatagctatttttttcttttttttataacaaacttaaaaatagtcagatatttaaactgttgtcgatttgcactctccaaaaagtttaaactgatggtgaagtgatcaaatgccaccatccccattggccagccccacaccagaaaggagcaaccaattggtggaatcttggataatgtatcttgatatacacaagacataatatttaccatttggggatatcatctgtgcatccacttgtgcgctttggagccaagccatgagagggaggggtgcatggccccccactagctatgaggaagaaaatggaaggaatctatgagggaacgacgacgatgaccatgacgatgaccatgacgatgacgatgatgtgcctctatgaggagttggaatccacatcggagatggagacacttgtatgcttgcaattattttgttccatcttaaaagatgaggggttggatgtgctagaatttgatgggtagattaaatgcatcgaaaaaactgaagatttttttttctttgaactagtatttgacggtagcagaaacttttttttccatgtgtgggactggttggttgcttgggttgggaattatcaaatggatttgaatcaaaattggacgggcaagttaaaagatgaaaaatgttactgacagattttatgtggaacaggggttgctttgggaacaatttgggggtttttccttaatcccttatccagatacaagccaatagtagtagcaacaaccgacctgaccgggatctaattaagtcaggtcgacattgaacatccttatcaatgccacttttacataaaggattatgaggctaagcaaatcaataatcacctctctcattgctaatggatgaactgttaattggtggggaattaaacagcgcatatgaatcaaatccacccatctattcgctgaattattttatccatctagtcttaaacaggattgtgccctagctagcatctatatttgcttatcatgatagactttcgttccaagcaattatcatgataaacatgccatgatcatgaatagttagccttaaagcgcgagaaaacacataattgaatggctggcctgttgttaatttttaggtggctccatggagccaatcatatatatgtgtaaatggtttcataggatattaccgtggatcaagcattaattgcatgggaaacattagaaaaaaaggagtacattttaagtttgatctcctgttcttaccaacttgaaccccataactttaccttttgttttactttgaacctctttccctcgaaatgttgccatctccaggaagacaaataaataagatgttattagtacggtgatggagacgagagtagcatggccagtaaagagagcgcgctagtacaccaagtagagcttaaggaaaacaataaaatacatagtgaaaaagagataaataaggggttcaaagtgaaagtatagattaaattgggtctaatgtgaaactctagtaaaaggaagcaggggcaggcccagaatttttgttgggtattcgaaatagaaagggataaaaaaaatacaatatcaagcctaataaatgatatgtatgtatagtttcgtatagttataaactatataatatataatggctataagcatattttcataaattcatagaattggccatgaaatgaattgaatgtattttaacatatagctgaatgggctaaattgtagcccaaactcagtgtgggctgagaggaaagggggaggagagtggaaaatttgggcctaatagagaaggaatggtgaaatagggtccaagataagtagtttaagacttttttatttttttaatacatatatgaagtatagagtatatatatatctattttttttctaaaaatcttgggtatacatctgaatacccttaatcactactaggcccgccgctgaaaggaagaggtccaaactgacattaactatttgaaaaaaaaaatattccagtttgccctcctcgtcttaccaaaatttcatgctataccacctttagccaatgcttttagtacaggaagttaacttaactccacaggtgttaatctgcctatccatcaaccatggtgaatagaaaaaggctctcactactccttgcttgtgggcgttgttgatcgacaactatacagaacaattttacatgatatagaaaattcaataaagaagcacatctaacttctacatcatggatacactgagccaatcaatcttcgagagaatgtagatgagaataagtgaatctattctttctataaagctaatacccactacactcctaaagctcaatatacaagcatagtatttattagcactactaaaacctgcatgcaagcatgccacatcacccaattaagctcacaaaactcaacatgcaaacacatagtaattatatctacaatttattttattctaaaactaaacatatatatgctaaattatcattctcaaattattttcataatatttcaatccaaatcattcatcatttctcgattatctaacatatatcccgcagcaaagcgcggggcatcatctagtaacactataacagagtataatcttgcagctaacacccactcggagtaggatcatcattgacatgttaagattgagatcctagttttttttttttgctatatatggtggattagcatgggtagagaagccgaggaagatgactaattaagctgtgttcgccattcttggttcctaacaggaggagtaagcacagaaaacggagcggtccattagcacgtgattaattaagtattagctaattttttttcaaaaatggattaatttgatctttttaagcaactttcgtatataaattttttttgcaaaaaacacatcattcagcagtttgaaaagcgtgcgcgcgaaaaatgagggagatgggttgggaaaaggggtgccgaacacaccctaatgatatctctgatatctcaaacaagagcgatgctccggtgctctctactgttagagggtaaattagtaatttactaatacaattattaggatttaccttttcatgtatgttttttttgggctaaatcgatctatgatcatccattgcactggcatcgctcatgtttcatcaaaaaagccaaaaaaagaaaaaaaaaggtttagtcactgctaaatggcgattatatcctccgcacctattttttcttacaataatacccctccacatttctactaccgctaattggggtggtagtataaatagatttgaacccttcgatcaaatgagattaacggttcagattaatttctactatcagtagagagcattgtagtgaggctcacatattgcttcacttcaaataaaatcaatagtactatgactataatatgagtatcatgtacaaaactagttaatggcaaagtgtgagtgtggcaagtggtgaccaatgaaggagaaatgggaataagaatgagctattacatatatagatacaaattttaacctaaacttattaaaataattaaacacctatgtaaaatctaaacattttgtgggtccctaaattaaaggtagtccctcttcttgacccgctaggctagaaggtctgtccctgaacctctttggcctgagtttgtccttgtcaatccttagcaatgcccaatgatcttagctaggaagggacagcgactaggtatgggcaggatagaaagcagagaaagacatgagggtatttgcactctattgaaagtttgatggcattcaactttggaaattcggtggaatatacccactagtgcatgcctatgaataacgtggattaattaaggttaattgcaaaatcagaatacacactctcttagaacctagacaaaaaagttctctctctaaagtatatagttgttacatttataagaggcacctccaatagaagtaggattaaaagtttacattttattttgtttgtgcgggtattctttggtccgtgtacctagggatgaaaatggtacgaaaactttccggattccggatctattttcaaaaatggaatttgtcggtcgaaaatttttcggaaacagaaacaaattcgaaaatattttctcagaaacagaatcgaatatgataagggcagtttccatcggaactcagaatcggtctgaaactttccgaaaaatttctcagaatttttagaattataaaggcctataccatagtctataattccttcccagcccaacccaatttagcatcctaattcttaagtcactcaattcagcccaacacaatacacacagtttaaacttgagaattggacttgacatgattgtgttttatgatgtaatgttgaaattagagacttgagaattttatttgatatagtttgaaccttagtttgaggggtttttgtattcctataaattttttgttaccgtatcagcgccggtccgttttcgctccgttttttgtttcaataatatctgattccaatttcatatccggggttttcagttccgattccgattaaaaaaataaaaacaaaaatgataaagatggtttccgtccgttttcatccctacttgtacctctaaacaacgggagaaaaactcaatatcgaccgtcctttttcttctaggtagttttggccgattgatatctttagtccccaaacggtgaaagtatayatgatacttaccgtagctgagcgaaaacctcatttaccttttttttaatttttaagctgttcaatcgatttaactcttttaatcttttgcctgtaagtagtgaaattactggcgcccattaggaagagctaagacrtcaacaaatgaaaaatgattcggtggccacaattaagtttttcaatggactaagcaccgatcaatgaacaatcctttcaaagctctaaaaggtatgctccaaacagtaaacactcgccaaatttgacattctttgtasctcttctccctattgtgatggtccaaatataaatgataggctaagaagaacaactttcctaccatttttggcgttatagtactatatgcatctctaaatatacgacrcttttgactttattaaattccgatgtttatgaaactktaaccatraccatatgtttctgttakaaataatgataatattcaatatatattaccttgttatgtattaattacttcccgtatgtgttatatctcgttaccttctgaaatagcgtaaatkaaaatttttgaatcagtatawttaatatkttatttagtaacaaattctccttcttagtacttctctagcaaggtaaatgtgtgaattccacctttatcaccagttaattcttttttatatggttatagcttactaaatgctctatccgtttcacaatgtaagtcattctaacattttccacatttatattgatgttaacgaatctagacatactccctccgaatcactagtgaattcgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaaggcataagtgtaaagccctgggtgcctaatgagtgagctactcacattaattgcgttgcgctccactgccgctttccaggtcggaaacctgtcgtgcccagctgcattatgaatcggccaacgccgcgggagaggccggttgcgtattgggcgctcttccgcttcctcgctcac

SEQ ID NO: 20 - DNA, 1895 bp, Oryza sativa
ccattgtttgtggcgtgcatgcgtcgtgaagatacagactaatctattggacgttcacaccttgttatacctaattttgatggccaatgactattataaggggtagggagggtcttgggtgtgaagcgacacgagtcgtaaatgagacagtcaacccaccctgtctaatgggtagatccgcccctaaagagtagtacgagtgaattatttagacatattctatcatcagttaggcccctttcgtttggaccggtagcttcgataacttctgtagcacgttttttaaatcgcttatataagaatttttttaaaaaataaatctattttataaatttaattgtaacaacgccttgggctgtgtttaattagaggcgttaggaacccctcccttcacacgtaaaatagagcgatgaattaacagacgattaattaagtattagcttaaaaagttttaaaatagactaatatgatttttatagaaacttttggaaaaacatatcatttagcaatttggaaaacgtgtgttcagaaaatgagagagatgaggttggaaacattgagggaggaacacaacccaaatagtttcaacgccacaaaaaagttctaatcctacataagggtttgcattatcctataacttcactttaaacctgctcatggtgttctttaggagttatagaaaccttccaaccccacctttggattgttgcacaactctactttaaactcctctcttgatttattcttagagcaattaatacagtgttctatatattgcctctaagatacaatataggattgggtgacaaagtgaatgagaaaagtgagagatgatgaaccattcgaaattaagagatrctccttccgtccttaaaaaaacgaatctatgactgaatatgacgcatcctacaaaaacgaatctgtataaaaatatatccagatttattctactaggatatatcacatcaagtggtaggttggtttttatgtgacggaggaaataacctccacataaacacggaagaacaatgtgtaactggatatgacagattttatggtaggaaacttttattttaacgcgattttaactattagaacacgataatatcatctttagggacaaaatacccctaaatattcgtgattttaaatctagaacacggtaaaaaaaataaaaatcaatctttagagacaaaatgattttaactatatatagaacacgataaaattcataaaaatcaatctttagggacaaaatatggttatttgctggttaactttatagtgtcctatatttaaaatcatgaaattgcagtgctcacttgctcttataagttgctactcgattaattcttagttgctaccaaacgtagctaatactaagtaattattaattaataataatgcgctaatcttttctgaagagttcgccaccaaacataccctaatactcgatcaatattgattgattatatccgtaatactcaatttattattgattaattatgcgctaatgacccgttcyttttttcagccgtcaatagctagagttgtgtgattcaaacgcaagcggccttaatttaatccgcttttaatttaggatatcagtcacaactcacaaatgtaaaaccagtaactaattaaacaccgattcatgaatattattagaagcagaacaagaaacactatgaccaagcattagtacctgatcatgcatctaacagtacagatcaacttgttaaactctatatcattaacctaatcctccgcttaattacctattaataatctctctctctctcggttcccttttgggaacattaacacaaagcctcagatcaggagcttccacacgcgcccaccgtgttgcccagcttcttcgaatcgcaatcgag

SEQ ID NO: 21 - DNA, 3990 bp, Oryza sativa
tcgctcgcctcgtcgcgcgtcctcttctccggcgccggcagcttccgcccccctgaccgcctggacgaccctgagcagcagcattacgccctccacgttgaggctccaccaccccgggcccgccggcgccgccgcccgccctccgcgccgcctcccgccgcggcgacgtacggctcgtctcgatggcctccagaaggtcggacacgaccgacttgcttggcagcgccgccgccgagccacaggcgccggcgctggtcccggcggcattattggcggcagcgccgccggcggcggcggcgagggtgatcatcaggtggcgcgcgggctggagcgcgccggcgaggagggagcggctgagcagcttgacgaccgccgacgtgcgtacggcgcgctccagctggctcgcgagtccgacaacggcgccggcgccggcgcgttcggccgccgcgtcggcggtggcctggccggcgccggcgggcggcgaggtgggcttcttcagacgctgtatcctggcgtgggcgcgcacccacgggttggagtgttggttcatatcgccgccggcttcgtcttcacctgaacgaaacatcgagaacaaaacgaagaaattaatcaggactcgacacgtctcactgcaacagtgttaaaactaatcactacttttagagcaagttttaatagtatgaggcaattgcatcggtgcactcactttcgaagccaattattgttttacccctactttttagggcttgcagttttaccgccacattttaaatctaaatcaaccatgtacctccatttctaacggccgattttaacggtgctagttgttaaaaaaaaggacatttatgtcctgggtggttaatatatttttgccctatttttttcagttttgcgtgattttgctcctaatttgtgatttgtgatttcagcttgtgtgatgagcattttgaccaaatataagcatctcttgtgatgttttttactttaacaaaaaagaatcacaaatataggcaattacaaaatttaaacaaaaatcccaaatactagccttcatggtcaatgctctagaccttgtgaggtgctgagagatgagagtcatgttaggcatcttggagtaagtatcaaacagattcgtagctcccgagcttggcagcgcaaggcttgccggatccagcggacgaagagccgctcccttccaggcactaacctcaggaaatacatgtagaaatggcagtgattcagaggcgcgttgtagctgcggagcgtgaaaacgcgatccacgaagctgatgaacctgtccaactcctgataaccccaccaatccatggagaggcccggtgcttccttccagtggtggagccaacgacgggcgagcacgcacgtcgccaccgcctcgtgcgccgggagcagcaacagcacgaggcggagcaccttgtccggcagggcgctgataacggtctggcggcacctcgtgcgtgtgggtggaagcgcggcattggggggcgcgcgcccgcggccgcctccgcctccgcctccgcacaatccggtgggagatggagataatggttggatgtttatcgatttgcctcgaggatagaacggtaaatcgatatttcataagattcattttttatttggtatttgtttttctaaaaaaggtgtgggacgcaacctcaatcggtgttaaaaagaaatgagggagttttaaataaaatgggggtaaaatggcaacaccctaaaaagtgagggtaaaacagcagttgaggttaaatgtaggggcatggatgcaactgtccctaaatagcataagccaaatgttagatctaaatcatccatatatattctgtcagtccattcatacaataatactgcatatacctacagtatctttcatgtatgacgctgttaactttttaacccctatttatccatctcctttagtaaattatttatgcaaacaaaaaatacttaagtcatgactaaacgcatcagatttcaatattataaaataatctttacataaatttttttagtaagacaaaagatcaaacgtttgttaaaaagtcaacggcgttatacattaaaatacggaggaagtaatatgttggacataacgtcttagaatccatgttacagctagctagatacaaatagcttgctttcttctctcttctcttttcatcgcatatcatattcggcttatagcctgctatgtacttgctctaactaatgtttttgatctagtacatgttgttgtgtacagtaatcaacctagtatatatgcacgcatgcatgaagataatttgatacgcatcgcacaatatgtaattaactagtaggattaattggtaatcacacgcgttctaatttgcatgttaattggaataccttgctgcgtggtggagagatcgtggtaggagcagaaccgggagaagatctcggcgagggtggcgcacttcctctcggcgccaccgccgccgacgccggcgtcggaagggacgacgacggcgtagaagaatccgggcatctcgaagacgaagacgcgggcgtcgaggcgcgcgacgagggcgtcccgggacagcatccagtggtggtcgccgacggcgcaggaggccatggagaccgggtggccctcgacgcgggccatgcgcaccgacaggccgccggagcggaacagccgcgccgcctcgaagcggtcgccgcacagcacgaagaggctgaccggcggcgagccctcagcggcgagcagctgcgtctccctccccatccggtactcctgcgtccgcccgactcgcagcggcggccgccctcctcctcctcctcctcctccctcccgagtcgccatctccgatcgatcaacacttaatgctgcttgatcacacaaattaagctactagattcaatttcactctgtactgcaaaagaagragaggaagaagaagaagctgcaggcacgcaaagagcatgtccatacggcaaatatactggycctttttattagacagtgatagatcgttggctcgtggctagrtagatatttgttgcccggatatttttccatgcagctaatattattcgctaggttaaacaacggcacgtgttaaggttaatgaccatgtatgtatgcaccactcactcaatgctagctaccagttaaactatggtacgtagtgcttaccacaacgagagccggatcggagagaaaagagaagggaattacgttagctagagagggatcatgttggagtatatgcgaggttgtacgagctgggagaagacatgagggtggccactcttaaataagagggaggtagtggtagtagtagatagccatggtcgccgaccgaggaagaagaagcagtggggggtcgcgaaacggaggccgcgcccgcgcgggagaagagagcgtattgcattgcattgcatgcatgcagtgcacgcgccacgcacgcgggacacctgcgcgccacatcgagcggggggtaggggcccaggcgtggggcccgcaggcgctacacgcggcccccaccgcagcgcgacgtggcccgacgacggccacgcgtcccgcagcgctggaccccttgcatgccgccgcctcgcctgcgccgcgaatccgcgaccgcttcgcgcgcgcgcgcgcgcgccgagccgagccgttcgagttcgcgcgctgcgcgccggtcgaatcgaataccggcggcattggcctgcctaatcaggggcttagagcaagcataatagacgtctctgtaagaaagtagacgagagaggaaagaaaagtgaggagaaacggtctatgaatttgtctataaatttatagctggtttaaaaaaaaaamccaaaaaataaatctatgaaagacagatggaccatatatttattatagtgaaaagctaacaactatataagtgggttaaaagatagactataaaaatatatataatcagtagctggctgtattattagctatgctcatagggcctgtctagg

SEQ ID NO: 22 - DNA, 3409 bp, Oryza sativa
ggaatagaaaaggtgggcaaagggtgaatagctaaagtggtccttcaagttttatcwaaagcttaagtttagttcttaatgtttcaatttatcccatattagtcctttaagttctaattttagttcaaacttatccttgaacccatttagaagccacatggctaaatataacaactttcttgataaataatacttatacccccctcattcacatatataagaaaaacaattgcattcaatgattgttttatagataatgcacagtaatttatgtaaaatgaaaataaattatgtacttacaagtgtatacgatagccattgtttaagtttaatgtgcacatgttgaaataatgagaaatatatctgcaatgttgtttattcatcttagttttatcttttctagtgtatagtttgtatacaaactaaagggcaaaagagacattctctaacataaatattgaatagaaatagccacatggcatattaagtgggtccaaagatgattttaaacaaaaataaatatttagacgatatatgtgaacaaaatgaratctaaagtatcaaattgagcatttgatgaaacttgagggactaaattagctattcacccgtgaggaaaataggaatrcatgcacactccaatatataggaatttttcaagagatttgagtggattgaaaattttctatgtttttttctatacaacgtgtggaaaagaaaaaaatcttttatttytactatatctacatttatattaaccgaatagtatccatagaaattaatcctgagaaatctaaatactttaaaatttatataaaacaaaaaagcccttaaattgtcgaactatatattcacgaaaaaaaagacaatataactagcatcaggatgttaaactcacaggacacagcaattatgatagttcgaatgaatttgggttcgttcaggtgtgtcaaargtctgaagaaacggctggaaccaagtcaggaaacaacgtatatttgtttccacccagaataatgcaagatctcccaaggccacattgccattcgtctgcccaagccccaagggtggggaacataactggaaagagtgcaccccctccgtcccataatataaataattttaattgtttgtttgtactattatctctcctcttattaaraagtttgtgtaaatataaaaaatgaaaagttatgtttaaagtagtttggataataaagtaggtcataaataaaataataataattctaaaaatttttattaagacgaatggtcaaacagagcatgtaaaaagttaaaatatcttatattataagaccgaatgagtagctgtgtagtacagtagttctggccggtcggcggtggcgacatgaaccgaattggttttggaagtactccgtatttgacgttactgtaaaattatattagctgtataagtaatttctggtttgtagatatgatgaaactggccactcatgttttagtttgtgttaattcaatctcagtctccactgattattttatttggtgaagacgagattaattgttgtacatgttttttagactaattaattaataatttgttaatgtttttcaaaataaatcggctagccgtcaatttaaatgaatagagtggaaaaaactgtatgcaaataaaaactgtaagaacatgtacaagttaggtcccgagatgtgggctagtgattaagataacaagtacatctgtttcacaatgtaagttattctagtatttttcacattatatgtctagattcaatatgaatttaagaaatgctagaatgacttatattgtgaaatggagagagtatgacttatattgtgaaatggagagagtagtagcgagttccctagccattgcaaggacccgtgttttgccctcttcttttatcttggttaggcgggatgttgagatctcttaataatgttaaaattgaaatttcttttgtattttttttcccatatctcttagaggactagcaaagctaatatacggactacttgctttgaggctgcacaattttttcatgtaaggccgacttgagttcttaatttttattcagactttcaacttttccatcacatcaaaacttttctatatacataaactttcaatttttttcttcaaactttcaattttagtcaaactttcaattttaacgtggaactaaacacaacctaaaactagcttctatcatgatcattcaaatttcatcgatcccatggtgaagcagtcaaagcagatcgttgctcggcgagcaagatgtactccccatacaaagaaaagtcttctcttacgcacgtagtagtacgtgtctatgtctcgcctgtatccagcccagcccagtacgtgcggagagaggagtcgattgttcttggaatctcggtgcgcgcgtagtttggctccttccaacaccgtatattcccccatgtctctcggaacccccccaatacagtagaacatctcgaccagaacatccctatgcgtttcgctgactggtggggtcccagaaggccaactcgaccaaaatattgggacaaaaagctacacacttcytctcaggtagcggtagcagccgtactttaattgcaacttgcatgaccttgggcccatgccctgcgtctgcatgcacgagtagtggaaagcgagagcccactacactactacgcggtgatgtcacgtacgcatgcgtcgcgccgcgccgcgsccatcggcgcaccggaaggccaggggccatcagccatgccatcttttgcgtggatcgatccaccccgcgaggccgccccgccctcccgcggtggtcggaggcggcgtacgtacgttacgcgagtagaggagatgcgtgggaggagcgggagcgcgacacgcgggcctcggcgcagcgacgcgtgcccgccgcgcgcggcagcgcgacrcacgtacgtgtagtagagctgggacgccgcgcgcacgactcgactcgagtgggctctcgatgggcggctttccgtttcgctgctcggtggaacatggaacgggcttggtccagcgtatggtagtgcagctgtattgggtgggtcaaaccaccaggggcaagttgttgtttccctgcgcgcgctagctttacggacgcgtggctcgttgggtgtctgcaactcacggagtagtggcgtgctgcctgagggtgcggtgtgccgcgtgcttcggtgcgtgcgatgcgactgtgattctgctctaggacagcttactccttcgtccaaaaaaaaatctcctatttaaaataagtataatttagtattataaatcaaaacagtattagaatatgtcacatctctaaaaacaagtaatataatacttaatgtgttatccataaaagatactaaatgagattggatgatgaagtggaggaga

SEQ ID NO: 23 - DNA, 20 bp, Artificial Sequence
  Description of Artificial Sequence Synthetic primer
tcactttggtgccattttca

SEQ ID NO: 24 - DNA, 20 bp, Artificial Sequence
  Description of Artificial Sequence Synthetic primer
tatgccgtggcttttaggac

SEQ ID NO: 25 - DNA, 20 bp, Artificial Sequence
  Description of Artificial Sequence Synthetic primer
catcatcactgatcggcaag

SEQ ID NO: 26 - DNA, 20 bp, Artificial Sequence
  Description of Artificial Sequence Synthetic primer
gctacaaccaccatgcacac

SEQ ID NO: 27 - DNA, 18 bp, Artificial Sequence
  Description of Artificial Sequence Synthetic primer
ctccccgagaggcacttc

SEQ ID NO: 28 - DNA, 20 bp, Artificial Sequence
  Description of Artificial Sequence Synthetic primer
caacttgagcaactccacga

SEQ ID NO: 29 - DNA, 20 bp, Artificial Sequence
  Description of Artificial Sequence Synthetic primer
ggcacacttcgccttaccta

SEQ ID NO: 30 - DNA, 20 bp, Artificial Sequence
  Description of Artificial Sequence Synthetic primer
ccatgactccatcccaaaac

SEQ ID NO: 31 - DNA, 20 bp, Artificial Sequence
  Description of Artificial Sequence Synthetic primer
acgggattagcatagggtca

SEQ ID NO: 32 - DNA, 20 bp, Artificial Sequence
  Description of Artificial Sequence Synthetic primer
ggcgatgagagagagagagc

SEQ ID NO: 33 - DNA, 20 bp, Artificial Sequence
  Description of Artificial Sequence Synthetic primer
ccgtgcagatgggattttag

SEQ ID NO: 34 - DNA, 20 bp, Artificial Sequence
  Description of Artificial Sequence Synthetic primer
ggtgaacccaccgatttatg

SEQ ID NO: 35 - DNA, 20 bp, Artificial Sequence
  Description of Artificial Sequence Synthetic primer
gttgttgtgaaggagcagca

SEQ ID NO: 36 - DNA, 20 bp, Artificial Sequence
  Description of Artificial Sequence Synthetic primer
catgacagacaggcagaagg

SEQ ID NO: 37 - DNA, 21 bp, Artificial Sequence
  Description of Artificial Sequence Synthetic primer
ggtacaaatgcctaacgcaaa

SEQ ID NO: 38 - DNA, 20 bp, Artificial Sequence
  Description of Artificial Sequence Synthetic primer
cgatggtcggatattctcgt

SEQ ID NO: 39 - DNA, 23 bp, Artificial Sequence
  Description of Artificial Sequence Synthetic primer
tgtttaaatcaatcattgggaat

SEQ ID NO: 40 - DNA, 20 bp, Artificial Sequence
  Description of Artificial Sequence Synthetic primer
gtccgctgcaaaggaaataa

SEQ ID NO: 41 - DNA, 21 bp, Artificial Sequence
  Description of Artificial Sequence Synthetic primer
cgagtgataatttgcatgctt

SEQ ID NO: 42 - DNA, 21 bp, Artificial Sequence
  Description of Artificial Sequence Synthetic primer
ttccacacatgattggctaca

SEQ ID NO: 43 - DNA, 20 bp, Artificial Sequence
  Description of Artificial Sequence Synthetic primer
agaggcagctttacgctcac

SEQ ID NO: 44 - DNA, 19 bp, Artificial Sequence
  Description of Artificial Sequence Synthetic primer
gtagctaggcgccaaaagg

SEQ ID NO: 45 - DNA, 20 bp, Artificial Sequence
  Description of Artificial Sequence Synthetic primer
acgaaagcataggcatccac

SEQ ID NO: 46 - DNA, 20 bp, Artificial Sequence
  Description of Artificial Sequence Synthetic primer
agggggtgaggttttgaatc

SEQ ID NO: 47 - DNA, 20 bp, Artificial Sequence
  Description of Artificial Sequence Synthetic primer
aaccacagcctacgcataca

SEQ ID NO: 48 - DNA, 20 bp, Artificial Sequence
  Description of Artificial Sequence Synthetic primer
cccaccctccttaaattcct

SEQ ID NO: 49 - DNA, 20 bp, Artificial Sequence
  Description of Artificial Sequence Synthetic primer
aggtactgcacccgaagaga

SEQ ID NO: 50 - DNA, 19 bp, Artificial Sequence
  Description of Artificial Sequence Synthetic primer
gatcctggtgtccaagcac

SEQ ID NO: 51 - DNA, 20 bp, Artificial Sequence
  Description of Artificial Sequence Synthetic primer
tacagcctcaaccgtgtcct

SEQ ID NO: 52 - DNA, 20 bp, Artificial Sequence
  Description of Artificial Sequence Synthetic primer
aatggtgtccacattgcaga

SEQ ID NO: 53 - DNA, 21 bp, Artificial Sequence
  Description of Artificial Sequence Synthetic primer
gcattgatcttcctcttgtgc

SEQ ID NO: 54 - DNA, 20 bp, Artificial Sequence
  Description of Artificial Sequence Synthetic primer
caacgtagcaacaggctgaa

SEQ ID NO: 55 - DNA, 20 bp, Artificial Sequence
  Description of Artificial Sequence Synthetic primer
tcgttggatccattgacaga

SEQ ID NO: 56 - DNA, 20 bp, Artificial Sequence
  Description of Artificial Sequence Synthetic primer
ctgtgtgctcgttgaccact

SEQ ID NO: 57 - DNA, 20 bp, Artificial Sequence
  Description of Artificial Sequence Synthetic primer
gcagcgtcgtacctaccttc

SEQ ID NO: 58 - DNA, 20 bp, Artificial Sequence
  Description of Artificial Sequence Synthetic primer
atcgatccatggtgcatttt

SEQ ID NO: 59 - DNA, 20 bp, Artificial Sequence
  Description of Artificial Sequence Synthetic primer
gcgtggattgtaggcatgta

SEQ ID NO: 60 - DNA, 20 bp, Artificial Sequence
  Description of Artificial Sequence Synthetic primer
acgactgtccacagaaacca

SEQ ID NO: 61 - DNA, 20 bp, Artificial Sequence
  Description of Artificial Sequence Synthetic primer
tggagagtgcaaatcgacaa

SEQ ID NO: 62 - DNA, 20 bp, Artificial Sequence
  Description of Artificial Sequence Synthetic primer
tggggatatcatctgtgcat

SEQ ID NO: 63 - DNA, 20 bp, Artificial Sequence
  Description of Artificial Sequence Synthetic primer
cacatccaacccctcatctt

SEQ ID NO: 64 - DNA, 20 bp, Artificial Sequence
  Description of Artificial Sequence Synthetic primer
gaatcaaaattggacgagca

SEQ ID NO: 65 - DNA, 20 bp, Artificial Sequence
  Description of Artificial Sequence Synthetic primer
tcaggtcggttgttgctact

SEQ ID NO: 66 - DNA, 21 bp, Artificial Sequence
  Description of Artificial Sequence Synthetic primer
ctgagccaatcaatcttcgag

SEQ ID NO: 67 - DNA, 21 bp, Artificial Sequence
  Description of Artificial Sequence Synthetic primer
tgcatgttgagttttgtgagc

SEQ ID NO: 68 - DNA, 20 bp, Artificial Sequence
  Description of Artificial Sequence Synthetic primer
gggtagagaagccgaggaag

SEQ ID NO: 69 - DNA, 20 bp, Artificial Sequence
  Description of Artificial Sequence Synthetic primer
cacgcttttcaaactgctga

SEQ ID NO: 70 - DNA, 20 bp, Artificial Sequence
  Description of Artificial Sequence Synthetic primer
gctccggtgctctctactgt

SEQ ID NO: 71 - DNA, 20 bp, Artificial Sequence
  Description of Artificial Sequence Synthetic primer
aggtgcggaggatataatcg

SEQ ID NO: 72 - DNA, 20 bp, Artificial Sequence
  Description of Artificial Sequence Synthetic primer
agtgtgagtgtggcaagtgg

SEQ ID NO: 73 - DNA, 20 bp, Artificial Sequence
  Description of Artificial Sequence Synthetic primer
ctcaggccaaagaggttcag

SEQ ID NO: 74 - DNA, 20 bp, Artificial Sequence
  Description of Artificial Sequence Synthetic primer
ttgatggcattcaactttgg

SEQ ID NO: 75 - DNA, 20 bp, Artificial Sequence
  Description of Artificial Sequence Synthetic primer
atccctaggtacacggacca

SEQ ID NO: 76 - DNA, 20 bp, Artificial Sequence
  Description of Artificial Sequence Synthetic primer
tagtccccaaacggtgaaag

SEQ ID NO: 77 - DNA, 20 bp, Artificial Sequence
  Description of Artificial Sequence Synthetic primer
gccaccgaatcatttttcat

SEQ ID NO: 78 - DNA, 21 bp, Artificial Sequence
  Description of Artificial Sequence Synthetic primer
ttcctaccatttttggcgtta

SEQ ID NO: 79 - DNA, 22 bp, Artificial Sequence
  Description of Artificial Sequence Synthetic primer
cgctatttcagaaggtaacgag

SEQ ID NO: 80 - DNA, 20 bp, Artificial Sequence
  Description of Artificial Sequence Synthetic primer
ttctgttgctggctgtcatc

SEQ ID NO: 81 - DNA, 20 bp, Artificial Sequence
  Description of Artificial Sequence Synthetic primer
ggtcgaggcaattatgcaat

SEQ ID NO: 82 - DNA, 20 bp, Artificial Sequence
  Description of Artificial Sequence Synthetic primer
ggtagatccgcccctaaaga

SEQ ID NO: 83 - DNA, 20 bp, Artificial Sequence
  Description of Artificial Sequence Synthetic primer
aggggttcctaacgcctcta

SEQ ID NO: 84 - DNA, 20 bp, Artificial Sequence
  Description of Artificial Sequence Synthetic primer
aaccccacctttggattgtt

SEQ ID NO: 85 - DNA, 20 bp, Artificial Sequence
  Description of Artificial Sequence Synthetic primer
cgtttttgtaggatgcgtca

SEQ ID NO: 86 - DNA, 20 bp, Artificial Sequence
  Description of Artificial Sequence Synthetic primer
cacaaagcctcagatcagga

SEQ ID NO: 87 - DNA, 18 bp, Artificial Sequence
  Description of Artificial Sequence Synthetic primer
gcgtaatgctgctgctca

SEQ ID NO: 88 - DNA, 19 bp, Artificial Sequence
  Description of Artificial Sequence Synthetic primer
cgtaatgctgctgctcagg

SEQ ID NO: 89 - DNA, 20 bp, Artificial Sequence
  Description of Artificial Sequence Synthetic primer
cggttcacttttgggaacat

SEQ ID NO: 90 - DNA, 20 bp, Artificial Sequence
  Description of Artificial Sequence Synthetic primer
cgtcttcgtcttcgagatgc

SEQ ID NO: 91 - DNA, 20 bp, Artificial Sequence
  Description of Artificial Sequence Synthetic primer
tttgatacgcatcgcacaat

SEQ ID NO: 92 - DNA, 20 bp, Artificial Sequence
  Description of Artificial Sequence Synthetic primer
atttgccgtatggacatgct

SEQ ID NO: 93 - DNA, 19 bp, Artificial Sequence
  Description of Artificial Sequence Synthetic primer
acagcacgaagaggctgac

SEQ ID NO: 94 - DNA, 20 bp, Artificial Sequence
  Description of Artificial Sequence Synthetic primer
tgcgactgtgattctgctct

SEQ ID NO: 95 - DNA, 23 bp, Artificial Sequence
  Description of Artificial Sequence Synthetic primer
tcatttctctcctccacttcatc

SEQ ID NO: 96 - DNA, 19 bp, Artificial Sequence
  Description of Artificial Sequence Synthetic primer
tccagctccttacggcttt

SEQ ID NO: 97 - DNA, 20 bp, Artificial Sequence
  Description of Artificial Sequence Synthetic primer
atatcggtcgacagcgagac

SEQ ID NO: 98 - DNA, 20 bp, Artificial Sequence
  Description of Artificial Sequence Synthetic primer
gatgcactccctctgttgct

SEQ ID NO: 99 - DNA, 21 bp, Artificial Sequence
  Description of Artificial Sequence Synthetic primer
tcaccttcttaccgaacacca

SEQ ID NO: 100 - DNA, 20 bp, Artificial Sequence
  Description of Artificial Sequence Synthetic primer
gttcaacgtggttgcacaat

SEQ ID NO: 101 - DNA, 19 bp, Artificial Sequence
  Description of Artificial Sequence Synthetic primer
gatgcaagcttggtcgtgt

SEQ ID NO: 102 - DNA, 32 bp, Artificial Sequence
  Description of Artificial Sequence Synthetic primer
gccctagggttcttgactggaaggtttctttg

SEQ ID NO: 103 - DNA, 30 bp, Artificial Sequence
  Description of Artificial Sequence Synthetic primer
gcggtaccttcaattgttacagcctcaacc

SEQ ID NO: 104 - DNA, 26 bp, Artificial Sequence
  Description of Artificial Sequence Synthetic primer
gcaagcttcggtcgtgcccctctcta

SEQ ID NO: 105 - DNA, 29 bp, Artificial Sequence
  Description of Artificial Sequence Synthetic primer
gccctaggtctagagtcgacctgcagaag

SEQ ID NO: 106 - DNA, 31 bp, Artificial Sequence
  Description of Artificial Sequence Synthetic primer
aagcttggtgtataaccgttgttccgtgagc

SEQ ID NO: 107 - DNA, 33 bp, Artificial Sequence
  Description of Artificial Sequence Synthetic primer
cctaggcaccaaagaaaccttccagtcaagaac

SEQ ID NO: 108 - DNA, 33 bp, Artificial Sequence; identical to SEQ ID NO: 107
  Description of Artificial Sequence Synthetic primer

SEQ ID NO: 109 - DNA, 34 bp, Artificial Sequence
  Description of Artificial Sequence Synthetic primer
gatggcgatcgccatggcgaaggcgagcgtggtg

SEQ ID NO: 110 - DNA, 36 bp, Artificial Sequence
  Description of Artificial Sequence Synthetic primer
ctaagtttaaaccagcctcaaccgtgtcctggacag

SEQ ID NO: 111 - DNA, 27 bp, Artificial Sequence
  Description of Artificial Sequence Synthetic primer
ccaccatgatgtagttcagttgtgaac

SEQ ID NO: 112 - DNA, 22 bp, Artificial Sequence
  Description of Artificial Sequence Synthetic primer
caccgttaaaatcggccgttag

SEQ ID NO: 113 - DNA, 27 bp, Artificial Sequence
  Description of Artificial Sequence Synthetic primer
tagttcccagataagggaattagggtt

SEQ ID NO: 114 - DNA, 27 bp, Artificial Sequence
  Description of Artificial Sequence Synthetic primer
ggtttcgctcatgtgttgagcatataa

SEQ ID NO: 115 - DNA, 27 bp, Artificial Sequence
  Description of Artificial Sequence Synthetic primer
cagtactaaaatccagatcccccgaat

SEQ ID NO: 116 - DNA, 27 bp, Artificial Sequence
  Description of Artificial Sequence Synthetic primer
acgtccgcaatgtgttattaagttgtc

SEQ ID NO: 117 - DNA, 15 bp, Artificial Sequence
  Description of Artificial Sequence Synthetic primer
ntcgastwtsgwgtt

SEQ ID NO: 118 - DNA, 16 bp, Artificial Sequence
  Description of Artificial Sequence Synthetic primer
ngtcgaswganawgaa

SEQ ID NO: 119 - DNA, 16 bp, Artificial Sequence
  Description of Artificial Sequence Synthetic primer
wgtgnagwancanaga

SEQ ID NO: 120 - DNA, 20 bp, Artificial Sequence
  Description of Artificial Sequence Synthetic primer
gctgctgctacacgtagtcg

SEQ ID NO: 121 - DNA, 20 bp, Artificial Sequence
  Description of Artificial Sequence Synthetic primer
gacactgacatgaaccgtgc

SEQ ID NO: 122 - DNA, 25 bp, Artificial Sequence
  Description of Artificial Sequence Synthetic primer
tcctctagagtcgagaattcagtac

SEQ ID NO: 123 - DNA, 24 bp, Artificial Sequence
  Description of Artificial Sequence Synthetic primer
agctgcatcatcgaaattgccgtc

SEQ ID NO: 124 - DNA, 24 bp, Artificial Sequence
  Description of Artificial Sequence Synthetic primer
tgtttatcggcactttgcatcggc

SEQ ID NO: 125 - DNA, 20 bp, Artificial Sequence
  Description of Artificial Sequence Synthetic primer
tccacgaccacaaggcaaac

SEQ ID NO: 126 - DNA, 19 bp, Artificial Sequence
  Description of Artificial Sequence Synthetic primer
tgcaatcgaccagcagcag

SEQ ID NO: 127 - DNA, 20 bp, Artificial Sequence
  Description of Artificial Sequence Synthetic primer
agcttaccgatgggcaccac

SEQ ID NO: 128 - DNA, 20 bp, Artificial Sequence
  Description of Artificial Sequence Synthetic primer
tcgcaggggaaattatcagg

SEQ ID NO: 129 - DNA, 18 bp, Artificial Sequence
  Description of Artificial Sequence Synthetic primer
gggaggatggtgttcgag

SEQ ID NO: 130 - DNA, 19 bp, Artificial Sequence
  Description of Artificial Sequence Synthetic primer
agcgagtcgaggaggaact

SEQ ID NO: 131 - DNA, 18 bp, Artificial Sequence
  Description of Artificial Sequence Synthetic primer
cagcagcgcctcatcttc

SEQ ID NO: 132 - DNA, 21 bp, Artificial Sequence
  Description of Artificial Sequence Synthetic primer
ggatgttgtagtcagccaagg

SEQ ID NO: 133 - DNA, 20 bp, Artificial Sequence
  Description of Artificial Sequence Synthetic primer
ctcaaggacctgcagaagga

SEQ ID NO: 134 - DNA, 19 bp, Artificial Sequence
  Description of Artificial Sequence Synthetic primer
atggacccatcagtgttgc

SEQ ID NO: 135 - DNA, 24 bp, Artificial Sequence
  Description of Artificial Sequence Synthetic primer
tgttgattatggaaagaagtccaa

SEQ ID NO: 136 - DNA, 25 bp, Artificial Sequence
  Description of Artificial Sequence Synthetic primer
gaggacactgttgtatggttctaca

SEQ ID NO: 137 - DNA, 21 bp, Artificial Sequence
  Description of Artificial Sequence Synthetic primer
aacaagggagtcctcttccag

SEQ ID NO: 138 - DNA, 18 bp, Artificial Sequence
  Description of Artificial Sequence Synthetic primer
cttgaacgcgtcctcgtc

SEQ ID NO: 139 - DNA, 20 bp, Artificial Sequence
  Description of Artificial Sequence Synthetic primer
ccactggacaaggaggtagg

SEQ ID NO: 140 - DNA, 25 bp, Artificial Sequence
  Description of Artificial Sequence Synthetic primer
tcaactcctctcccacgaggattcg

SEQ ID NO: 141 - DNA, 21 bp, Artificial Sequence
  Description of Artificial Sequence Synthetic primer
ttttagcagtactccgactgc

SEQ ID NO: 142 - DNA, 18 bp, Artificial Sequence
  Description of Artificial Sequence Synthetic primer
ttctaaagcgggtggtgg

SEQ ID NO: 143 - DNA, 18 bp, Artificial Sequence
  Description of Artificial Sequence Synthetic primer
agcaagtcggtcgtgtcc

SEQ ID NO: 144 - DNA, 18 bp, Artificial Sequence
  Description of Artificial Sequence Synthetic primer
gaccctgagcagcagcat

SEQ ID NO: 145 - DNA, 24 bp, Artificial Sequence
  Description of Artificial Sequence Synthetic primer
atgaagaccatgtgtcactgtcac

SEQ ID NO: 146 - DNA, 25 bp, Artificial Sequence
  Description of Artificial Sequence Synthetic primer
aattctgctactaccaactccagag

SEQ ID NO: 147 - DNA, 27 bp, Artificial Sequence
  Description of Artificial Sequence Synthetic primer
ccaccatgatgtagttcagttgtgaac

SEQ ID NO: 148 - DNA, 23 bp, Artificial Sequence
  Description of Artificial Sequence Synthetic primer
attggcttcgaaagtgagtgcac

SEQ ID NO: 149 - DNA, 22 bp, Artificial Sequence
  Description of Artificial Sequence Synthetic primer
caccgttaaaatcggccgttag

SEQ ID NO: 150 - DNA, 27 bp, Artificial Sequence
  Description of Artificial Sequence Synthetic primer
aattaggagcaaaatcacgcaaaactg

SEQ ID NO: 151 - DNA, 20 bp, Artificial Sequence
  Description of Artificial Sequence Synthetic primer
tcgatggcctccagaaggtc

SEQ ID NO: 152 - DNA, 21 bp, Artificial Sequence
  Description of Artificial Sequence Synthetic primer
cggcttcgtcttcacctgaac

SEQ ID NO: 153 - DNA, 21 bp, Artificial Sequence
  Description of Artificial Sequence Synthetic primer
ggcgttccctccttcttatgg

SEQ ID NO: 154 - DNA, 24 bp, Artificial Sequence
  Description of Artificial Sequence Synthetic primer
gcattggggcagtgttgttgtatg

SEQ ID NO: 155 - DNA, 693 bp, Oryza sativa
gctcaataaaatcatcaagccataatgaagaccatgtgtcactgtcacaataatcaaaccaatccaccttttcatccaaataaattctgctactaccaactccagagaaaaaaccaccatgatgtagttcagttgtgaactgttcatcctcgcagcctacatcataagacaaaccattctgatgtgtcaaatcgtaaataattatctgatctattatacacaaaaagattatacagagtacatattcatgcttagagcaaaaattatttctaatcacaaactattcgtgaaggattacttatatgacacctagccttataaaacaccaaagcagtggcggaggcagcgccgggccaccacaggcaattgcccaggctccatgcatacaacaacactgccccaatgcatatacaacatattactcattaatcatatacttaatcactttgttcataggttataatggagattaattagatgaataaacagaaacgaggaagcaaggaccaaaccatcaaactgtagctacagagaccaaactatacaatcacaagattcaagaaaaaacagagaaacatgcaattaccggtacaaatcaatgaaaacctaactttatcaacaacaatcaattgaaccaaaaacgaaaatatgtaatcactagggagttatttaccataagaaggagggaacgcc

SEQ ID NO: 156 - DNA, 750 bp, Oryza sativa
cgtacggctcgtctcgatggcctccagaaggtcggacacgaccgacttgcttggcagcgccgccgccgagccacaggcgccggcgctggtcccggcggcattattggcggcagcgccgccggcggcggcggcgagggtgatcatcaggtggcgcgcgggctggagcgcgccggcgaggagggagcggctgagcagcttgacgaccgccgacgtgcgtacggcgcgctccagctggctcgcgagtccgacaacggcgccggcgccggcgcgttcggccgccgcgtcggcggtggcctggccggcgccggcgggcggcgaggtgggcttcttcagacgctgtatcctggcgtgggcgcgcacccacgggttggagtgttggttcatatcgccgccggcttcgtcttcacctgaacgaaacatcgagaacaaaacgaagaaattaatcaggactcgacacgtctcactgcaacagtgttaaaactaatcactacttttagagcaagttttaatagtatggggcaattgcatcggtgcactcactttcgaagccaattattgttttacccctactttttagggcttgcagttttaccgccacattttaaatctaaatcaaccatgtacctccatttctaacggccgattttaacggtgctagttgttaaaaaaaaaggacatttatgtcctgggtggttaatatatttttgccctatttttttcagttttgcgtgattttgctcctaatttgtg

SEQ ID NO: 157 - DNA, 24 bp, Artificial Sequence
  Description of Artificial Sequence Synthetic primer
atgaagaccatgtgtcactgtcac

SEQ ID NO: 158 - DNA, 25 bp, Artificial Sequence
  Description of Artificial Sequence Synthetic primer
aattctgctactaccaactccagag

SEQ ID NO: 159 - DNA, 27 bp, Artificial Sequence
  Description of Artificial Sequence Synthetic primer
ccaccatgatgtagttcagttgtgaac

SEQ ID NO: 160 - DNA, 23 bp, Artificial Sequence
  Description of Artificial Sequence Synthetic primer
attggcttcgaaagtgagtgcac

SEQ ID NO: 161 - DNA, 22 bp, Artificial Sequence
  Description of Artificial Sequence Synthetic primer
caccgttaaaatcggccgttag

SEQ ID NO: 162 - DNA, 27 bp, Artificial Sequence
  Description of Artificial Sequence Synthetic primer
aattaggagcaaaatcacgcaaaactg

SEQ ID NO: 163 - DNA, 20 bp, Artificial Sequence
  Description of Artificial Sequence Synthetic primer
tcgatggcctccagaaggtc

SEQ ID NO: 164 - DNA, 21 bp, Artificial Sequence
  Description of Artificial Sequence Synthetic primer
cggcttcgtcttcacctgaac

SEQ ID NO: 165 - DNA, 21 bp, Artificial Sequence
  Description of Artificial Sequence Synthetic primer
ggcgttccctccttcttatgg

SEQ ID NO: 166 - DNA, 24 bp, Artificial Sequence
  Description of Artificial Sequence Synthetic primer
gcattggggcagtgttgttgtatg